SEQUENCE LISTING (116 sequences)

SEQ ID NO: 1 - DNA, 20 bp, synthetic construct
  source 1..20: mol_type = other DNA
tatcactgacggagcacaga

SEQ ID NO: 2 - DNA, 20 bp, synthetic construct
  source 1..20: mol_type = other DNA
tatcacttacggatcacaga

SEQ ID NO: 3 - DNA, 21 bp, synthetic construct
  source 1..21: mol_type = other DNA
gtatcactgacggagcacaga

SEQ ID NO: 4 - DNA, 21 bp, synthetic construct
  source 1..21: mol_type = other DNA
gtatcacttacggatcacaga

SEQ ID NO: 5 - DNA, 76 bp, synthetic construct
  source 1..76: mol_type = other DNA
gttttagagctagaaatagcaagttaaaataaggctagtccgttatcaacttgaaaaagtggcaccgagtcggtgc

SEQ ID NO: 6 - DNA, 97 bp, synthetic construct
  source 1..97: mol_type = other DNA
gtatcacttacggatcacagagttttagagctagaaatagcaagttaaaataaggctagtccgttatcaacttgaaaaagtggcaccgagtcggtgc

SEQ ID NO: 7 - DNA, 97 bp, synthetic construct
  source 1..97: mol_type = other DNA
gtatcactgacggagcacagagttttagagctagaaatagcaagttaaaataaggctagtccgttatcaacttgaaaaagtggcaccgagtcggtgc

SEQ ID NO: 8 - DNA, 6655 bp, Homo sapiens
  source 1..6655: mol_type = genomic DNA
agcagatccgcggaagggcagaatgggactccaagcctggtgagaaattgagagggctcgggagaaagggatcacgttggagggagcacacattgggagggtgggaacacaaggacaacgtagctcccactgaaacccacctgctgcccctacagcctcctagggctctttgccctcatcctctctggcaaatgcagttacagcccggagcccgaccagcggaggacgtgagttgacttagcacagactgcccccttccccataccctgttctgcctcccactgtcctggtcctagcttctctcacccccgtgccagctcctagtacgcactccatagcttcatctctatgttcctagcctcagtcccctatcattcacctcatgtgatctgtatctgctcctaggatcctccccaaggtctcagctcctaatctggaaccttccatgaccaatattttccatctccaccctaaccaaagccatgtccctgacccctgaccctacaggctgcccccaggctgggtgtccctgggccgtgcggaccctgaggaagagctgagtctcacctttgccctgagacagcagaatgtggaaagactctcggagctggtgcaggctgtgtcggatcccagctctcctcaatacggtgccttttgggactgaggacaggatgtgggatgcggtggagggacacagggctgggttgggcatgggatggcgatcatgtcagagcctgccaagacacttgtgttcctcaggctagaaacctaaaaggggatgtggttcagtatacaggctttatgatcaaatacgaactcaaattctgactctgctacttactagctaccaggcatctagtacaacttacgttccttcccctaccctcaaatccattctaatttcctcttttgttcacatactaaggctgccagttctaactcccaaagagcctttggattaatctccttcttgccgtcttttgttacgaccaactccgttatttattcccactcctggactactgcccagctccccagctgatctgcagtctcctccctccaccccacccctcactgtactcccccactctgcttcaaaacagtctctccaacagtcaaaatggattggttccttctcctggttaaagcccttcacagcagggggagtgtgtgcttgtgaactgcagaggctggggacggggcagtgtgacactagtgtgcatggcaggaaacagtgactcaccatcgtgttaagcttaaaatcaacaagtatgcaaatttgagctaaactgaataactggtaccgaaagatttgaaaacatttaccggacataagcttaaaattaaaatggaaaatttatatgtattaacctcattcattttcatgatcatggcaatacatgctgtggtgcgacatttgcaacttactggcctttagggtaaatccatattattggccatggcatttcaagtctctccagccttttcttcctctgcttcccaagtacaccctacacctgcacacatagccctcctttaccctgttccaggctttgggaagtcctgatgtctcatagttgaggtccaaaagggggagtttgggaaagcaatgaatgagggcaagtgcctcttctgaatccctgcaggaaaatacctgaccctagagaatgtggctgatctggtgaggccatccccactgaccctccacacggtgcaaaaatggctcttggcagccggagcccagaagtgccattctgtgatcacacaggactttctgacttgctggctgagcatccggtgagaggaaatgattgctccatggagggcaccagtcatcccatcagtgagatggatgggagggagttgagagcttgctggggcttgtgggtgggagctaatgcatggggagacagtgactgactgcccagggatgctcagaggtagcttcttctgttccgttttgagctttctgacctctgttctctgacctccagacaagcagagctgctgctccctggggctgagtttcatcactatgtgggaggacctacggaaacccatgttgtaaggtccccacatccctaccagcttccacaggccttggccccccatgtggactttggtaacacctatggggtgaatgggggttggggcacacagatccaggggctgaggaagtttagatgccattggggactgggggtggggtgagttgtaaggtgggcattacagtctataagatctcctcaagcctgacttctccctacagtggggggactgcaccgttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccaggtgagccaagcaaagagccccagggtcctcatagcctccccacagtgtcctcaattccttaccaccctgggactcaccctcggacccacgatctctgctctgactccctccatagttcctggagcagtatttccatgactcagacctggctcagttcatgcgcctcttcggtggcaactttgcacatcaggcatcagtagcccgtgtggttggacaacagggccggggccgggccgggattgaggccagtctagatgtgcagtacctgatgagtgctggtgccaacatctccacctgggtctacagtagccctggtactaccaagaggactggacagtggggaagggggtgggagatgggtgttgatccctgctccctcaagggaatgctataagctggagagagatcctgacaacccccagtgactatctttgtgcccatccctcaaaaaaaaaaaaaaaaaaaatccaggccggcatgagggacaggagcccttcctgcagtggctcatgctgctcagtaatgagtcagccctgccacatgtgcatactgtgagctatggagatgatgaggactccctcagcagcgcctacatccagcgggtcaacactgagctcatgaaggctgccgctcggggtctcaccctgctcttcgcctcaggtgacctcctaccctaaacttagacaatgcttacacctctgcagcctgggagctttgactccacagtgatccctgagcctggtctctgactcataatctgaactcagaccttccagtagggaccactgacctgacctctacactctgacctcctacagtaacaaatttcccctctgacatccgaacccacatactaagccctaaccaattaatatgaatgctacacttggtctctctcaggtgacagtggggccgggtgttggtctgtctctggaagacaccagttccgccctaccttccctgcctccaggtaagtactctagcctaccactcaggtataaccaccacctttcacttgtgatctcatgatgtagaacctttgtcttgaccccaccatgtgctcctgtggttcagccttaagctttgcctgccctggttgctgtactcctgtctcttcttcctgcaggtcccaggccccaaatctcttgtgtgggatacagctcccattgttccttttcgtcagttcccaggcattttagtggaagatttggtgggtgttctgtagagaaaagtgtgcacagtcacctcgggccatgccttgaaggctcaaaatctcttagtcaatcccatatacatgcttccccacagagtctagttcctccagcaagacctgggctatactcacccctccccacatatcttggaggtccccttgggtcccctactatccaaatgctgtcttctcccctcagcccctatgtcaccacagtgggaggcacatccttccaggaacctttcctcatcacaaatgaaattgttgactatatcagtggtggtggcttcagcaatgtgttcccacggccttcataccaggtacgtgtgtttgtgtggatggatgcagggtaagagtgaggatgggggatcctcagttcagctgactgctgggcaggccacatgccaatactcactcaaaaatgcctttcaggaggaagctgtaacgaagttcctgagctctagcccccacctgccaccatccagttacttcaatgccagtggccgtgcctacccagatgtggctgcactttctgatggctactgggtggtcagcaacagagtgcccattccatgggtgtccggaacctcggtgagaatcagcccatctccaaactctcactcaggaactacccttaccccctaacaccttgaacaccttgcacctagaacccctgactccttagagatgtctgatactttaaagcatcactcccaaaaagtccaatcactcagaacccctgacctctacttgcaccttcactcttgtaggcctctactccagtgtttggggggatcctatccttgatcaatgagcacaggatccttagtggccgcccccctcttggctttctcaacccaaggctctaccagcagcatggggcaggactctttgatgtaagtatggaagggaagggtgtggacgttttcaaacaactatggggagtgctaagggggacttgggggcagttagggtggtgtggaatagcctttgaaatgtgagtacagggtgaggagatatactctttaagtactggtactagtaggcccagatctgatgccagcctcctccctaggtaacccgtggctgccatgagtcctgtctggatgaagaggtagagggccagggtttctgctctggtcctggctgggatcctgtaacaggctggggaacacccaacttcccagctttgctgaagactctactcaacccctgaccctttcctatcaggagagatggcttgtcccctgccctgaagctggcagttcagtcccttattctgccctgttggaagccctgctgaaccctcaactattgactgctgcagacagcttatctccctaaccctgaaatgctgtgagcttgacttgactcccaaccctaccatgctccatcatactcaggtctccctactcctgccttagattcctcaataagatgctgtaactagcattttttgaatgcctctccctccgcatctcatctttctcttttcaatcaggcttttccaaagggttgtatacagactctgtgcactatttcacttgatattcattccccaattcactgcaaggagacctctactgtcaccgtttactctttcctaccctgacatccagaaacaatggcctccagtgcatacttctcaatctttgctttatggcctttccatcatagttgcccactccctctccttacttagcttccaggtcttaacttctctgactactcttgtcttcctctctcatcaatttctgcttcttcatggaatgctgaccttcattgctccatttgtagatttttgctcttctcagtttactcattgtcccctggaacaaatcactgacatctacaaccattaccatctcactaaataagactttctatccaataatgattgatacctcaaatgtaagatgcgtgatactcaacatttcatcgtccaccttcccaaccccaaacaattccatctcgtttcttcttggtaaatgatgctatgctttttccaaccaagccagaaacctgtgtcatcttttcaccccaccttcaatcaacaagtcctcaatcaacaagtcctactgactgcacatcttaaatatatctttatcagtccacaagtccttccaattatatttcccaagtatatctagaacttatccacttatatccccactgctactaccttagtttagggctatattctcttgaaaaaaagtgtccttacttcctgccaatccccaagtcatcttccagagtaaaatgcaaatcccatcaggccacttggatgaaaacccttcaaggattactggatagaattcaggctttcccctccagcccccaatcatagctcacaaaccttccttgctatttgttcttaagtaaaaaatcatttttcctcctccctccccaaaccccaaggaactctcactcttgctcaagctgttccgtccccttaccacccctgatacaactgccaggttaatttccagaattcttgcaagactcagttcagaagtcaccttctttcgtgaatgttttgattccctgaggctactttattttggtatggctgaaaaatcctagattttctaaacaaaacctgtttgaatcttggttctgatatggactaggagagagactgggtcaagtaagcttatctccctgaggctgtttcctcgtctgttaagtgtgaatatcaatacctgcctttcataatcaccagggaataaagtggaataatgttgataacagtgcttggcacctggaagtaggtggcagatgttaacgcccttcctcccttgcactgcgccccctgtgcctacctctagcattgtaacgaccacgtagtattgaaatggccagtttacttgtctgccttcctttccaagaccgttggtgcctagaggactagaatcgtgtcctatttaactttgtgttcccaggtcctagctcaggagttggcaaataagaattaaatgtctgctacaccgaaaa

SEQ ID NO: 9 - protein, 563 aa, Homo sapiens
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 10 - DNA, 7472 bp, Mus musculus
  source 1..7472: mol_type = genomic DNA
gcaacgtcactagttactaggcagggagatgggggaggtgccagaccatgctcatgtgacttatcacatgactacagatcagcctgaaagccaaaatgggactccaagcccggtgagaaactgaggtggggagggtagctaagggggattacattagagggattacatgttgtagaaggagtacaatgagggttcggctcctgatgaaacccacctgctgtccctgcagcctcctagggctccttgctctcgtcatcgccggcaaatgcacttacaaccctgagccggaccagcggtggatgtgagttgatttagtactacaggtgcctgtctctgcagacgcttggtcctagctttcctcatcctcaactgctcctagtacccactctgtagccttattcgacaccccctcggcttaatctctggtcatttatctcctatgatccttatctgcatctaggattcgacccaaagtctcagttcttaatcccaaactttccatgatctggatttctcgttatggcctaatcaaatcatgtccctgaccctgtaggctgcctccaggctgggtgtccctgggccgcgtggatcccgaggaagagctgagtctcacttttgcgctgaaacagcggaacctggaaagactctcggagctggtgcaggctgtgtcggatcctagctctcctcaatatggtgcctatcagttggggcaggaagtgggaggcaggaccaggcaggcactggctggtgcagatcatgtagggccatgtttctggtatgacagacaaatgacaataactgccatattcatacacacacacacacacacacacacacacacacacacacaccaaaatttccacctgagttcttcagtcttaaaactaagaacaaatctaagcctagtagcatgaggtaataatcctagctacttggactaaaacaggacgattttgaatccaaggcatgcctgggcagtgtagcaatattgtcaacattaaaaccaattttttttagaggcctagctagctcattgatagggtgggtgcttacggagcatatgaaaaaccctaggttcaagtgccaggaagatggtgggtgtggggtagctagctgtgtttaagaacacatgattcataatcagatctagattgaaatgttcacttaccctaccagctctcaaatggaggactagcacttagtgtgtgtgcatgatcaagtcatggaccttaccccactctcatagccactgccacaccctcctctgttgctagttcaagtcctaaattgtctttggattaatccactctggccattcctcttgctaccaactcagttgtttattctctttctggattactgcccagtacccggactggtttgaagcttcctctccttcaccccactccccagaacagtcccttctctattctagagtattctctatggtgctcaggctggagtgaatccttcccctgcttaaagtcctgtatggtgaggcaagggtgtgtgtgtgtgtgtgtgtgtgtagaatggaggcgagaggaggggggggcatccttagagcacccaagaagctcctcatatgcaggaaagaatgactcaccgttatgttgtttaaaatcaacaaggatgaagagctgagctaaattagaaggttctgtataactggctccaaaagtttggggaaatctttttgaagataagcttaaaatgggatattgaaagtttactaacgtccttgagtttttgtgaccatggtaacacatgctgtgcatttttcctggccatgacgtttcatgtctctcgagccttttcttccctttgactctcactatgcctagccctacacagtcctcctcgggaagttttgatgtcctctgattgaggtccagacagacaacccaggaaagcctcaaatgaggacgagagcctcttctgaatccctgcaggaaagtacctaaccctggaggatgtagctgagctggttcaaccatcacccctgaccctcctcactgtccaaaagtggctctcagcagctggagcccggaactgcgattcagtgaccacccaggactttctgacttgctggctgagtgtccggtgagaagtaatgatttccccatagaatccattgtccctacaaggagacaaaataccccaattgggggagtttaaagcgtgctgggagcctgtgggtataggtaatgcatacggaaatgatggcaggctgctaagtggggctcaggccaactcttctactccttcccatggtttctgttttcttacttccagacaggctgagctgctgctcccaggagctgagtttcatcgctatgtagggggacctacaaagacccatgttataaggtccccacatccctaccagcttccccaggccttggcccctcatgtggattttggtaaacccaatggagttggtggaagttgtgggagggaggtctacaggctgaagaattttagatgccaaaagaggcatagaatctttccagtagagaagtgggtggtagtgtctgtaagacctccttaagcctgacccctttccacagtgggggggctgcaccgtttccccccttcatctccaagacaacgtccagaaccacaacaggtaggaactgttagcctgcacttgggagtgactccgtctgtgctccgtcagcgatacaacctgacagccaaagatgtgggctcaggcaccaccaacaatagccaggcctgtgcccaggtgagccatgtaaagccctgcggtcatcacaacctcctcaagatatctttagtgcctcactaccctgggcctcactctctgatccacaattcctgatttggatgtttccacagttcctggaacagtacttccataactcggatctgactgagttcatgcgcctattcggtggcagttttacacaccaggcctcagtagcaaaagttgttggaaagcaagggcgaggccgagctgggatcgaggccagtctagatgtggaatacctgatgagtgctggtgccaatatctccacttgggtctacagtagccctggtattgctaagagaattagttgggggatgggaaaatgggttggagtagacttttggtctctgcttcatttcatcaaggggatgccatgggctgaagggagattctagcaaccatccaatggccattcatatcccttcttttaaaacaattcaggccgccatgaggcacaggagcccttcttacaatggctcctgcttcttagcaatgagtcatctttgccacatgtacatactgtgagttacggagacgatgaagactccctcagcagcatctacatccagagagtcaacactgagttcatgaaggctgctgctcggggtctcaccctcctttttgcctcaggtaaccttctaccataaatttaagacttcccacctacccaagcggcagactttatcccaaccgacccttcagcctggtttctgactcataataggaactcagagccttaacagggtctgctgatttgacctgagtactctgaggtggcatatacagttcttctctggtatatagaagcctacaccccaagttcttcacaactaatctgaatacttactatgtctttcccaggtgacactggagctgggtgttggtctgtctccggaagacacaagttccgccctagcttccctgcttccaggtaagtaccccactctttcacttgtgacagaggccaccaggagctgctgtggctcagccttcagcattacctgttgtgtttgctgtgctcccttttcttcctgcgtatcccaggctgcgagagcagattatggcgtttcttttccttagttgctagggtttgttgcttgtttttcatgtagaaaagtatatacaattaactccagccatgtcttgagagctcccaatctatcaataaactctgtatacaggcttctatagtcttactcccttttccagtaagacccagaccattcccacccacctccacacatcttggaggtcacccattgtcttagtcggggtttttattgtattgctgcgatgaaacaccgtgacaaaaaaacaaaacaaaacaaaacaaaacaaaaacagttgaggaggaaagggtttatttggcttacacttccagatcacatccgtcactgaaggaaatcaggacagaaactcaagcagggctggaacctggaagcagaagctgatgaagaggccagggaatggtgctgcttactggcttgcttcccatggcttgttcagcctgccatcttatagaacccacgaccatcagcccagggatgccaccctccacaatgggctgggccctccctcattgatcactaattgagaaaatgtcctacagctggatctcatggaggcatttccttaactgagcttcctttgtctctgatgactcttgtatcaagttgacaacacaaaactaaccagcaagtacattcactatctgaatactgtcttctcctcagcccctatgttactacagttggaggaacctccttcaagaatcctttcctcatcacagatgaagtagttgactatatcagtggtggaggcttcagcaatgttttcccacggcctccctaccaggtttgtggatattcctgtggatatctggaggttgaaggtgatgggtggggctcagtcctgcagcttgctgagcaggctgctggccaatactcatactcagaaatgtccttcaggaggaagcagtggcccagttcttgaaatccagctctcatctaccaccatccagttacttcaatgctagtggccgtgcctacccagatgttgccgcactatctgatggctactgggtggtcagcaacatggtccccattccatgggtatctggaacctcggtaagaatcagctctgctctaaacgctctactcaggaactaccctcgctcctccacctacacaatctaaacgctctactcaggaactaccctcgctcctccacctacacaatctaaacgctctactcaggaactaccctcgctcctccacctacacaatctaaacgctctactcaggaactaccctcgctcctccacctacacaatcttgaacccagaacccccgactccttggagactcctgatctttgcaagcatcatcccttagaagtccaatccctctaaaaccctaacctattcttgcatcttcatcttgcaggcctctactccagtgtttgggggaattttatccttgataaatgagcacagaatcctcaatggccgccctcctcttggctttctcaaccccaggctctatcagcagcatgggacaggactctttgatgtgagtattggaggaaagagtgtggatgttgtcataggatatgagaagggctctggtgaacttcggcattttcactatctatgattgcctctgtgatatgactataaataaggtgcagtctaggagctggtaccagcaggcccagacctgatgccatcatctcctcccaggtaacccacggctgccatgagtcctgtctgaatgaagaagtggagggtcagggtttctgctctggtcctggctgggatcctgtgacaggttggggaacacccaacttcccagccctactgaagaccctgctcaacccttgaccctttcgtgccatgacgagaaagcagaactgttccctgtactaaaagggaaggctcagtttcttgttattcctcgatagaagccctgctgaactcctgttgcctgctgcagatagcttctccctaaccctcagatgctgtgaacaggactcaactctcaatcctactgtgtgccatcaaactcaggtctccaaacttctacttcaagatcctcaacaagatgctataaccagcatattttgtctcaccccaaccccatctctccttcctctttccagcttgagatgtgaaagcagggcaagaaggttcagtcttccattactgacactagcaggtccacccaacgcttaccacctctgcactgaccgtacactctatttctcttcgggtttgcttttccgttcactgaagtgagacctttgactaatcgttttgtctttcttctctcggcactgaagtacaatggtctccccaatgttttatccagttatacccttttcagtgtttgttttatgggttttcttatttaagaacaggttgtcaaaaaaccattaaaaaaaaaaaagaaaaagaacacattggctgcaatctatttaactatataactattctaaggaaagttaaaaattgaaaacttaaaatgtttgaaatgttctcattggcaaaattcctcaacaaaataaataggtcattacaaattttgctttaaatttttgcttgagtgatttttttttttgtaaagtgttttaaaatttacattttattttccccttctcagctacaccaaagcaacattcaggttttaatttcaactgtcagacattacaaacatctagcttctgtgaacctgggtgtttgttctttccatcagtttcccatttatcctttttcctcagtgacccccctctgaccacactaatcagcccccttttctgctgactccagggatgctgtgttcattgttccattcttagtttcttgctcttatcattttattatcatcccttgacagatcactgacatctatacccacagtgagtgatacctcaaatgtgagatacctcgttacttcatctccctccagcccagacctaaactatttcatctcttaatctatgatataatgcctctttcaaacaagccagaaacctataactcttaactctcatctttttcacctccttactaacttcaaaaagttttcttgactgcctctcaagtatatctttatcggctagtgtggtgatgcacacctttaatcccagcactcaggagacagaggccaggcaagtctctgagtttgaggccatcctggtctatataaagagttccagacaagccaggcctacatagtcagaccatgtctcaaaaatacacatatgtgcacacacacacatgcacaaaatactgcattatctttgagcccatgttcttttttcttctaaactgcaggaagcacttggggagaggaaaggcttccgaagcccctcatcttccaaaccaagcagtctatgtatttgtgcaaacccttcaaagattactgggtaaaagtcagagacattgaaacttgccttcaaaatcgggaaataaacattgctaatgtctcacacttggaattaagcaatgaatgttagtttccctttcttctttgcactgcacctacatgtagctgggaaacaatcatacagtattgaaattgtcagtttgtttgccttccttttccagacagtcggtgctagtgaactagaatctgattcttaactttgtattcctaggtcccagcccaaatagaaattaaataaatgactgtttaaaaaaaaa

SEQ ID NO: 11 - protein, 562 aa, Mus musculus
MGLQARLLGLLALVIAGKCTYNPEPDQRWMLPPGWVSLGRVDPEEELSLTFALKQRNLERLSELVQAVSDPSSPQYGKYLTLEDVAELVQPSPLTLLTVQKWLSAAGARNCDSVTTQDFLTCWLSVRQAELLLPGAEFHRYVGGPTKTHVIRSPHPYQLPQALAPHVDFVGGLHRFPPSSPRQRPEPQQVGTVSLHLGVTPSVLRQRYNLTAKDVGSGTTNNSQACAQFLEQYFHNSDLTEFMRLFGGSFTHQASVAKVVGKQGRGRAGIEASLDVEYLMSAGANISTWVYSSPGRHEAQEPFLQWLLLLSNESSLPHVHTVSYGDDEDSLSSIYIQRVNTEFMKAAARGLTLLFASGDTGAGCWSVSGRHKFRPSFPASSPYVTTVGGTSFKNPFLITDEVVDYISGGGFSNVFPRPPYQEEAVAQFLKSSSHLPPSSYFNASGRAYPDVAALSDGYWVVSNMVPIPWVSGTSASTPVFGGILSLINEHRILNGRPPLGFLNPRLYQQHGTGLFDVTHGCHESCLNEEVEGQGFCSGPGWDPVTGWGTPNFPALLKTLLNP

SEQ ID NO: 12 - protein, 1368 aa, Streptococcus pyogenes [sequence omitted]

SEQ ID NO: 13 - protein, 1368 aa, Streptococcus pyogenes
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 14 - protein, 1368 aa, Streptococcus pyogenes
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 15 - protein, 1367 aa, synthetic construct
MDKKYSIGLDIGTNSVGWAVITDDYKVPSKKFKVLGNTDRHSIKKNLIGALLFGSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQIYNQLFEENPINASRVDAKAILSARLSKSRRLENLIAQLPGEKRNGLFGNLIALSLGLTPNFKSNFDLAEDAKLQLSKDTYDDDLDNLLAQIGDQYADLFLAAKNLSDAILLSDILRVNSEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGAYHDLLKIIKDKDFLDNEENEDILEDIVLTLTLFEDRGMIEERLKTYAHLFDDKVMKQLKRRRYTGWGRLSRKLINGIRDKQSGKTILDFLKSDGFANRNFMQLIHDDSLTFKEDIQKAQVSGQGHSLHEQIANLAGSPAIKKGILQTVKIVDELVKVMGHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 16 - protein, 1404 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSSDYKDHDGDYKDHDIDYKDDDDKAAG

SEQ ID NO: 17 - protein, 1378 aa, Lactobacillus fermentum
MKEYHIGLDIGTSSIGWAVTDSQFKLMRIKGKTAIGVRLFEEGKTAAERRTFRTTRRRLKRRKWRLHYLDEIFAPHLQEVDENFLRRLKQSNIHPEDPTKNQAFIGKLLFPDLLKKNERGYPTLIKMRDELPVEQRAHYPVMNIYKLREAMINEDRQFDLREVYLAVHHIVKYRGHFLNNASVDKFKVGRIDFDKSFNVLNEAYEELQNGEGSFTIEPSKVEKIGQLLLDTKMRKLDRQKAVAKLLEVKVADKEETKRNKQIATAMSKLVLGYKADFATVAMANGNEWKIDLSSETSEDEIEKFREELSDAQNDILTEITSLFSQIMLNEIVPNGMSISESMMDRYWTHERQLAEVKEYLATQPASARKEFDQVYNKYIGQAPKERGFDLEKGLKKILSKKENWKEIDELLKAGDFLPKQRTSANGVIPHQMHQQELDRIIEKQAKYYPWLATENPATGERDRHQAKYELDQLVSFRIPYYVGPLVTPEVQKATSGAKFAWAKRKEDGEITPWNLWDKIDRAESAEAFIKRMTVKDTYLLNEDVLPANSLLYQKYNVLNELNNVRVNGRRLSVGIKQDIYTELFKKKKTVKASDVASLVMAKTRGVNKPSVEGLSDPKKFNSNLATYLDLKSIVGDKVDDNRYQTDLENIIEWRSVFEDGEIFADKLTEVEWLTDEQRSALVKKRYKGWGRLSKKLLTGIVDENGQRIIDLMWNTDQNFKEIVDQPVFKEQIDQLNQKAITNDGMTLRERVESVLDDAYTSPQNKKAIWQVVRVVEDIVKAVGNAPKSISIEFARNEGNKGEITRSRRTQLQKLFEDQAHELVKDTSLTEELEKAPDLSDRYYFYFTQGGKDMYTGDPINFDEISTKYDIDHILPQSFVKDNSLDNRVLTSRKENNKKSDQVPAKLYAAKMKPYWNQLLKQGLITQRKFENLTKDVDQNIKYRSLGFVKRQLVETRQVIKLTANILGSMYQEAGTEIIETRAGLTKQLREEFDLPKVREVNDYHHAVDAYLTTFAGQYLNRRYPKLRSFFVYGEYMKFKHGSDLKLRNFNFFHELMEGDKSQGKVVDQQTGELITTRDEVAKSFDRLLNMKYMLVSKEVHDRSDQLYGATIVTAKESGKLTSPIEIKKNRLVDLYGAYTNGTSAFMTIIKFTGNKPKYKVIGIPTTSAASLKRAGKPGSESYNQELHRIIKSNPKVKKGFEIVVPHVSYGQLIVDGDCKFTLASPTVQHPATQLVLSKKSLETISSGYKILKDKPAIANERLIRVFDEVVGQMNRYFTIFDQRSNRQKVADARDKFLSLPTESKYEGAKKVQVGKTEVITNLLMGLHANATQGDLKVLGLATFGFFQSTTGLSLSEDTMIVYQSPTGLFERRICLKDI

SEQ ID NO: 18 - protein, 1053 aa, Staphylococcus aureus
MGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKK

SEQ ID NO: 19 - protein, 1409 aa, Streptococcus thermophilus
MLFNKCIIISINLDFSNKEKCMTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 20 - protein, 1402 aa, Lactobacillus crispatus
MKIKNYNLALTPSTSAVGHVEVDDDLNILEPVHHQKAIGVAKFGEGETAEARRLARSARRTTKRRANRINHYFNEIMKPEIDKVDPLMFDRIKQAGLSPLDERKEFRTVIFDRPNIASYYHNQFPTIWHLQKYLMITDEKADIRLIYWALHSLLKHRGHFFNTTPMSQFKPGKLNLKDDMLALDDYNDLEGLSFAVANSPEIEKVIKDRSMHKKEKIAELKKLIVNDVPDKDLAKRNNKIITQIVNAIMGNSFHLNFIFDMDLDKLTSKAWSFKLDDPELDTKFDAISGSMTDNQIGIFETLQKIYSAISLLDILNGSSNVVDAKNALYDKHKRDLNLYFKFLNTLPDEIAKTLKAGYTLYIGNRKKDLLAARKLLKVNVAKNFSQDDFYKLINKELKSIDKQGLQTRFSEKVGELVAQNNFLPVQRSSDNVFIPYQLNAITFNKILENQGKYYDFLVKPNPAKKDRKNAPYELSQLMQFTIPYYVGPLVTPEEQVKSGIPKTSRFAWMVRKDNGAITPWNFYDKVDIEATADKFIKRSIAKDSYLLSELVLPKHSLLYEKYEVFNELSNVSLDGKKLSGGVKQILFNEVFKKTNKVNTSRILKALAKHNIPGSKITGLSNPEEFTSSLQTYNAWKKYFPNQIDNFAYQQDLEKMIEWSTVFEDHKILAKKLDEIEWLDDDQKKFVANTRLRGWGRLSKRLLTGLKDNYGKSIMQRLETTKANFQQIVYKPEFREQIDKISQAAAKNQSLEDILANSYTSPSNRKAIRKTMSVVDEYIKLNHGKEPDKIFLMFQRSEQEKGKQTEARSKQLNRILSQLKADKSANKLFSKQLADEFSNAIKKSKYKLNDKQYFYFQQLGRDALTGEVIDYDELYKYTVLHIIPRSKLTDDSQNNKVLTKYKIVDGSVALKFGNSYSDALGMPIKAFWTELNRLKLIPKGKLLNLTTDFSTLNKYQRDGYIARQLVETQQIVKLLATIMQSRFKHTKIIEVRNSQVANIRYQFDYFRIKNLNEYYRGFDAYLAAVVGTYLYKVYPKARRLFVYGQYLKPKKTNQENQDMHLDSEKKSQGFNFLWNLLYGKQDQIFVNGTDVIAFNRKDLITKMNTVYNYKSQKISLAIDYHNGAMFKATLFPRNDRDTAKTRKLIPKKKDYDTDIYGGYTSNVDGYMLLAEIIKRDGNKQYGFYGVPSRLVSELDTLKKTRYTEYEEKLKEIIKPELGVDLKKIKKIKILKNKVPFNQVIIDKGSKFFITSTSYRWNYRQLILSAESQQTLMDLVVDPDFSNHKARKDARKNADERLIKVYEEILYQVKNYMPMFVELHRCYEKLVDAQKTFKSLKISDKAMVLNQILILLHSNATSPVLEKLGYHTRFTLGKKHNLISENAVLVTQSITGLKENHVSIKQML

SEQ ID NO: 21 - protein, 1385 aa, unidentified
  source 1..1385: note = Pedicoccus damnosus
MTNEKYSIGLDIGTSSIGFAVVNDNNRVIRVKGKNAIGVRLFDEGKAAADRRSFRTTRRSFRTTRRRLSRRRWRLKLLREIFDAYITPVDEAFFIRLKESNLSPKDSKKQYSGDILFNDRSDKDFYEKYPTIYHLRNALMTEHRKFDVREIYLAIHHIMKFRGHFLNATPANNFKVGRLNLEEKFEELNDIYQRVFPDESIEFRTDNLEQIKEVLLDNKRSRADRQRTLVSDIYQSSEDKDIEKRNKAVATEILKASLGNKAKLNVITNVEVDKEAAKEWSITFDSESIDDDLAKIEGQMTDDGHEIIEVLRSLYSGITLSAIVPENHTLSQSMVAKYDLHKDHLKLFKKLINGMTDTKKAKNLRAAYDGYIDGVKGKVLPQEDFYKQVQVNLDDSAEANEIQTYIDQDIFMPKQRTKANGSIPHQLQQQELDQIIENQKAYYPWLAELNPNPDKKRQQLAKYKLDELVTFRVPYYVGPMITAKDQKNQSGAEFAWMIRKEPGNITPWNFDQKVDRMATANQFIKRMTTTDTYLLGEDVLPAQSLLYQKFEVLNELNKIRIDHKPISIEQKQQIFNDLFKQFKNVTIKHLQDYLVSQGQYSKRPLIEGLADEKRFNSSLSTYSDLCGIFGAKLVEENDRQEDLEKIIEWSTIFEDKKIYRAKLNDLTWLTDDQKEKLATKRYQGWGRLSRKLLVGLKNSEHRNIMDILWITNENFMQIQAEPDFAKLVTDANKGMLEKTDSQDVINDLYTSPQNKKAIRQILLVVHDIQNAMHGQAPAKIHVEFARGEERNPRRSVQRQRQVEAAYEKVSNELVSAKVRQEFKEAINNKRDFKDRLFLYFMQGGIDIYTGKQLNIDQLSSYQIDHILPQAFVKDDSLTNRVLTNENQVKADSVPIDIFGKKMLSVWGRMKDQGLISKGKYRNLTMNPENISAHTENGFINRQLVETRQVIKLAVNILADEYGDSTQIISVKADLSHQMREDFELLKNRDVNDYHHAFDAYLAAFIGNYLLKRYPKLESYFVYGDFKKFTQKETKMRRFNFIYDLKHCDQVVNKETGEILWTKDEDIKYIRHLFAYKKILVSHEVREKRGALYNQTIYKAKDDKGSGQESKKLIRIKDDKETKIYGGYSGKSLAYMTIVQITKKNKVSYRVIGIPTLALARLNKLENDSTENNGELYKIIKPQFTHYKVDKKNGEIIETTDDFKIVVSKVRFQQLIDDAGQFFMLASDTYKNNAQQLVISNNALKAINNTNITDCPRDDLERLDNLRLDSAFDEIVKKMDKYFSAYDANNFREKIRNSNLIFYQLPVEDQWENNKITELGKRTVLTRILQGLHANATTTDMSIFKIKTPFGQLRQRSGISLSENAQLIYQSPTGLFERRVQLNKIK

SEQ ID NO: 22 - protein, 1367 aa, unidentified
  source 1..1367: note = Fusobaterium nucleatum
MKKQKFSDYYLGFDIGTNSVGWCVTDLDYNVLRFNKKDMWGSRLFEEAKTAAERRVQRNSRRRLKRRKWRLNLLEEIFSNEILKIDSNFFRRLKESSLWLEDKSSKEKFTLFNDDNYKDYDFYKQYPTIFHLRNELIKNPEKKDIRLVYLAIHSIFKSRGHFLFEGQNLKEIKNFETLYNNLIAFLEDNGINKIIDKNNIEKLEKIVCDSKKGLKDKEKEFKEIFNSDKQLVAIFKLSVGSSVSLNDLFDTDEYKKGEVEKEKISFREQIYEDDKPIYYSILGEKIELLDIAKTFYDFMVLNNILADSQYISEAKVKLYEEHKKDLKNLKYIIRKYNKGNYDKLFKDKNENNYSAYIGLNKEKSKKEVIEKSRLKIDDLIKNIKGYLPKVEEIEEKDKAIFNKILNKIELKTILPKQRISDNGTLPYQIHEAELEKILENQSKYYDFLNYEENGIITKDKLLMTFKFRIPYYVGPLNSYHKDKGGNSWIVRKEEGKILPWNFEQKVDIEKSAEEFIKRMTNKCTYLNGEDVIPKDTFLYSEYVILNELNKVQVNDEFLNEENKRKIIDELFKENKKVSEKKFKEYLLVKQIVDGTIELKGVKDSFNSNYISYIRFKDIFGEKLNLDIYKEISEKSILWKCLYGDDKKIFEKKIKNEYGDILTKDEIKKINTFKFNNWGRLSEKLLTGIEFINLETGECYSSVMDALRRTNYNLMELLSSKFTLQESINNENKEMNEASYRDLIEESYVSPSLKRAIFQTLKIYEEIRKITGRVPKKVFIEMARGGDESMKNKKIPARQEQLKKLYDSCGNDIANFSIDIKEMKNSLISYDNNSLRQKKLYLYYLQFGKCMYTGREIDLDRLLQNNDTYDIDHIYPRSKVIKDDSFDNLVLVLKNENAEKSNEYPVKKEIQEKMKSFWRFLKEKNFISDEKYKRLTGKDDFELRGFMARQLVNVRQTTKEVGKILQQIEPEIKIVYSKAEIASSFREMFDFIKVRELNDTHHAKDAYLNIVAGNVYNTKFTEKPYRYLQEIKENYDVKKIYNYDIKNAWDKENSLEIVKKNMEKNTVNITRFIKEKKGQLFDLNPIKKGETSNEIISIKPKVYNGKDDKLNEKYGYYKSLNPAYFLYVEHKEKNKRIKSFERVNLVDVNNIKDEKSLVKYLIENKKLVEPRVIKKVYKRQVILINDYPYSIVTLDSNKLMDFENLKPLFLENKYEKILKNVIKFLEDNQGKSEENYKFIYLKKKDRYEKNETLESVKDRYNLEFNEMYDKFLEKLDSKDYKNYMNNKKYQELLDVKEKFIKLNLFDKAFTLKSFLDLFNRKTMADFSKVGLTKYLGKIQKISSNVLSKNELYLLEESVTGLFVKKIKL

SEQ ID NO: 23 - protein, 1286 aa, Enterococcus cecorum
RRKQRIQILQELLGEEVLKTDPGFFHRMKESRYVVEDKRTLDGKQVELPYALFVDKDYTDKEYYKQFPTINHLIVYLMTTSDTPDIRLVYLALHYYMKNRGNFLHSGDINNVKDINDILEQLDNVLETFLDGWNLKLKSYVEDIKNIYNRDLGRGERKKAFVNTLGAKTKAEKAFCSLISGGSTNLAELFDDSSLKEIETPKIEFASSSLEDKIDGIQEALEDRFAVIEAAKRLYDWKTLTDILGDSSSLAEARVNSYQMHHEQLLELKSLVKEYLDRKVFQEVFVSLNVANNYPAYIGHTKINGKKKELEVKRTKRNDFYSYVKKQVIEPIKKKVSDEAVLTKLSEIESLIEVDKYLPLQVNSDNGVIPYQVKLNELTRIFDNLENRIPVLRENRDKIIKTFKFRIPYYVGSLNGVVKNGKCTNWMVRKEEGKIYPWNFEDKVDLEASAEQFIRRMTNKCTYLVNEDVLPKYSLLYSKYLVLSELNNLRIDGRPLDVKIKQDIYENVFKKNRKVTLKKIKKYLLKEGIITDDDELSGLADDVKSSLTAYRDFKEKLGHLDLSEAQMENIILNITLFGDDKKLLKKRLAALYPFIDDKSLNRIATLNYRDWGRLSERFLSGITSVDQETGELRTIIQCMYETQANLMQLLAEPYHFVEAIEKENPKVDLESISYRIVNDLYVSPAVKRQIWQTLLVIKDIKQVMKHDPERIFIEMAREKQESKKTKSRKQVLSEVYKKAKEYEHLFEKLNSLTEEQLRSKKIYLYFTQLGKCMYSGEPIDFENLVSANSNYDIDHIYPQSKTIDDSFNNIVLVKKSLNAYKSNHYPIDKNIRDNEKVKTLWNTLVSKGLITKEKYERLIRSTPFSDEELAGFIARQLVETRQSTKAVAEILSNWFPESEIVYSKAKNVSNFRQDFEILKVRELNDCHHAHDAYLNIVVGNAYHTKFTNSPYRFIKNKANQEYNLRKLLQKVNKIESNGVVAWVGQSENNPGTIATVKKVIRRNTVLISRMVKEVDGQLFDLTLMKKGKGQVPIKSSDERLTDISKYGGYNKATGAYFTFVKSKKRGKVVRSFEYVPLHLSKQFENNNELLKEYIEKDRGLTDVEILIPKVLINSLFRYNGSLVRITGRGDTRLLLVHEQPLYVSNSFVQQLKSVSSYKLKKSENDNAKLTKTATEKLSNIDELYDGLLRKLDLPIYSYWFSSIKEYLVESRTKYIKLSIEEKALVIFEILHLFQSDAQVPNLKILGLSTKPSRIRIQKNLKDTDKMSIIHQSPSGIFEHEIELTSL

SEQ ID NO: 24 - protein, 1312 aa, unidentified
  source 1..1312: note = Anaerostipes hadrus
MQNGFLGITVSSEQVGWAVTNPKYELERASRKDLWGVRLFDKAETAEDRRMFRTNRRLNQRKKNRIHYLRDIFHEEVNQKDPNFFQQLDESNFCEDDRTVEFNFDTNLYKNQFPTVYHLRKYLMETKDKPDIRLVYLAFSKFMKNRGHFLYKGNLGEVMDFENSMKGFCESLEKFNIDFPTLSDEQVKEVRDILCDHKIAKTVKKKNIITITKVKSKTAKAWIGLFCGCSVPVKVLFQDIDEEIVTDPEKISFEDASYDDYIANIEKGVGIYYEAIVSAKMLFDWSILNEILGDHQLLSDAMIAEYNKHHDDLKRLQKIIKGTGSRELYQDIFINDVSGNYVCYVGHAKTMSSADQKQFYTFLKNRLKNVNGISSEDAEWIDTEIKNGTLLPKQTKRDNSVIPHQLQLREFELILDNMQEMYPFLKENREKLLKIFNFVIPYYVGPLKGVVRKGESTNWMVPKKDGVIHPWNFDEMVDKEASAECFISRMTGNCSYLFNEKVLPKNSLLYETFEVLNELNPLKINGEPISVELKQRIYEQLFLTGKKVTKKSLTKYLIKNGYDKDIELSGIDNEFHSNLKSHIDFEDYDNLSDEEVEQIILRITVFEDKQLLKDYLNREFVKLSEDERKQICSLSYKGWGNLSEMLLNGITVTDSNGVEVSVMDMLWNTNLNLMQILSKKYGYKAEIEHYNKEHEKTIYNREDLMDYLNIPPAQRRKVNQLITIVKSLKKTYGVPNKIFFKISREHQDDPKRTSSRKEQLKYLYKSLKSEDEKHLMKELDELNDHELSNDKVYLYFLQKGRCIYSGKKLNLSRLRKSNYQNDIDYIYPLSAVNDRSMNNKVLTGIQENRADKYTYFPVDSEIQKKMKGFWMELVLQGFMTKEKYFRLSRENDFSKSELVSFIEREISDNQQSGRMIASVLQYYFPESKIVFVKEKLISSFKRDFHLISSYGHNHLQAAKDAYITIVVGNVYHTKFTMDPAIYFKNHKRKDYDLNRLFLENISRDGQIAWESGPYGSIQTVRKEYAQNHIAVTKRVVEVKGGLFKQMPLKKGHGEYPLKTNDPRFGNIAQYGGYTNVTGSYFVLVESMEKGKKRISLEYVPVYLHERLEDDPGHKLLKEYLVDHRKLNHPKILLAKVRKNSLLKIDGFYYRLNGRSGNALILTNAVELIMDDWQTKTANKISGYMKRRAIDKKARVYQNEFHIQELEQLYDFYLDKLKNGVYKNRKNNQAELIHNEKEQFMELKTEDQCVLLTEIKKLFVCSPMQADLTLIGGSKHTGMIAMSSNVTKADFAVIAEDPLGLRNKVIYSHKGEK

SEQ ID NO: 25 - protein, 1344 aa, Kandleria vitulina
MSQNNNKIYNIGLDIGDASVGWAVVDEHYNLLKRHGKHMWGSRLFTQANTAVERRSSRSTRRRYNKRRERIRLLREIMEDMVLDVDPTFFIRLANVSFLDQEDKKDYLKENYHSNYNLFIDKDFNDKTYYDKYPTIYHLRKHLCESKEKEDPRLIYLALHHIVKYRGNFLYEGQKFSMDVSNIEDKMIDVLRQFNEINLFEYVEDRKKIDEVLNVLKEPLSKKHKAEKAFALFDTTKDNKAAYKELCAALAGNKFNVTKMLKEAELHDEDEKDISFKFSDATFDDAFVEKQPLLGDCVEFIDLLHDIYSWVELQNILGSAHTSEPSISAAMIQRYEDHKNDLKLLKDVIRKYLPKKYFEVFRDEKSKKNNYCNYINHPSKTPVDEFYKYIKKLIEKIDDPDVKTILNKIELESFMLKQNSRTNGAVPYQMQLDELNKILENQSVYYSDLKDNEDKIRSILTFRIPYYFGPLNITKDRQFDWIIKKEGKENERILPWNANEIVDVDKTADEFIKRMRNFCTYFPDEPVMAKNSLTVSKYEVLNEINKLRINDHLIKRDMKDKMLHTLFMDHKSISANAMKKWLVKNQYFSNTDDIKIEGFQKENACSTSLTPWIDFTKIFGKINESNYDFIEKIIYDVTVFEDKKILRRRLKKEYDLDEEKIKKILKLKYSGWSRLSKKLLSGIKTKYKDSTRTPETVLEVMERTNMNLMQVINDEKLGFKKTIDDANSTSVSGKFSYAEVQELAGSPAIKRGIWQALLIVDEIKKIMKHEPAHVYIEFARNEDEKERKDSFVNQMLKLYKDYDFEDETEKEANKHLKGEDAKSKIRSERLKLYYTQMGKCMYTGKSLDIDRLDTYQVDHIVPQSLLKDDSIDNKVLVLSSENQRKLDDLVIPSSIRNKMYGFWEKLFNNKIISPKKFYSLIKTEFNEKDQERFINRQIVETRQITKHVAQIIDNHYENTKVVTVRADLSHQFRERYHIYKNRDINDFHHAHDAYIATILGTYIGHRFESLDAKYIYGEYKRIFRNQKNKGKEMKKNNDGFILNSMRNIYADKDTGEIVWDPNYIDRIKKCFYYKDCFVTKKLEENNGTFFNVTVLPNDTNSDKDNTLATVPVNKYRSNVNKYGGFSGVNSFIVAIKGKKKKGKKVIEVNKLTGIPLMYKNADEEIKINYLKQAEDLEEVQIGKEILKNQLIEKDGGLYYIVAPTEIINAKQLILNESQTKLVCEIYKAMKYKNYDNLDSEKIIDLYRLLINKMELYYPEYRKQLVKKFEDRYEQLKVISIEEKCNIIKQILATLHCNSSIGKIMYSDFKISTTIGRLNGRTISLDDISFIAESPTGMYSKKYKL

SEQ ID NO: 26 - protein, 1288 aa, Enterococcus faecalis
MRLFEEGHTAEDRRLKRTARRRISRRRNRLRYLQAFFEEAMTDLDENFFARLQESFLVPEDKKWHRHPIFAKLEDEVAYHETYPTIYHLRKKLADSSEQADLRLIYLALAHIVKYRGHFLIEGKLSTENTSVKDQFQQFMVIYNQTFVNGESRLVSAPLPESVLIEEELTEKASRTKKSEKVLQQFPQEKANGLFGQFLKLMVGNKADFKKVFGLEEEAKITYASESYEEDLEGILAKVGDEYSDVFLAAKNVYDAVELSTILADSDKKSHAKLSSSMIVRFTEHQEDLKKFKRFIRENCPDEYDNLFKNEQKDGYAGYIAHAGKVSQLKFYQYVKKIIQDIAGAEYFLEKIAQENFLRKQRTFDNGVIPHQIHLAELQAIIHRQAAYYPFLKENQEKIEQLVTFRIPYYVGPLSKGDASTFAWLKRQSEEPIRPWNLQETVDLDQSATAFIERMTNFDTYLPSEKVLPKHSLLYEKFMVFNELTKISYTDDRGIKANFSGKEKEKIFDYLFKTRRKVKKKDIIQFYRNEYNTEIVTLSGLEEDQFNASFSTYQDLLKCGLTRAELDHPDNAEKLEDIIKILTIFEDRQRIRTQLSTFKGQFSAEVLKKLERKHYTGWGRLSKKLINGIYDKESGKTILDYLVKDDGVSKHYNRNFMQLINDSQLSFKNAIQKAQSSEHEETLSETVNELAGSPAIKKGIYQSLKIVDELVAIMGYAPKRIVVEMARENQTTSTGKRRSIQRLKIVEKAMAEIGSNLLKEQPTTNEQLRDTRLFLYYMQNGKDMYTGDELSLHRLSHYDIDHIIPQSFMKDDSLDNLVLVGSTENRGKSDDVPSKEVVKDMKAYWEKLYAAGLISQRKFQRLTKGEQGGLTLEDKAHFIQRQLVETRQITKNVAGILDQRYNAKSKEKKVQIITLKASLTSQFRSIFGLYKVREVNDYHHGQDAYLNCVVATTLLKVYPNLAPEFVYGEYPKFQTFKENKATAKAIIYTNLLRFFTEDEPRFTKDGEILWSNSYLKTIKKELNYHQMNIVKKVEVQKGGFSKESIKPKGPSNKLIPVKNGLDPQKYGGFDSPVVAYTVLFTHEKGKKPLIKQEILGITIMEKTRFEQNPILFLEEKGFLRPRVLMKLPKYTLYEFPEGRRRLLASAKEAQKGNQMVLPEHLLTLLYHAKQCLLPNQSESLAYVEQHQPEFQEILERVVDFAEVHTLAKSKVQQIVKLFEANQTADVKEIAASFIQLMQFNAMGAPSTFKFFQKDIERARYTSIKEIFDATIIYQSPTGLYETRRKVVD

SEQ ID NO: 27 - protein, 1052 aa, Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 28 - protein, 1082 aa, Geobacillus thermodenitrificans
MKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL

SEQ ID NO: 29 - protein, 1375 aa, Streptococcus canis
MEKKYSIGLDIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDHIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGD

SEQ ID NO: 30 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 31 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARNAKTGAAGSLMDVLHHPGMNHRVEITEGILADECAALLSDFFRMRRQEIKAQKKAQSSTD

SEQ ID NO: 32 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARGAKTGAAGSLMDVLHHPGMNHRVEITEGILADECAALLSDFFRMRRQEIKAQKKAQSSTD

SEQ ID NO: 33 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARVAKTGAAGSLMDVLHHPGMNHRVEITEGILADECAALLSDFFRMRRQEIKAQKKAQSSTD

SEQ ID NO: 34 - protein, 166 aa, synthetic construct
SEVEFSYEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARNAKTGAAGSLMDVLHHPGMSHRVEITEGILADECAALLSDFFRMRRQEIKAQKKAQSSTD

SEQ ID NO: 35 - protein, 166 aa, synthetic construct
SEVEFSYEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARNAKTGAAGSLMDVLHHPGMSHRVEITEGILADECAALLSDFFRMRRQDIKAQKKAQSSTD

SEQ ID NO: 36 - protein, 166 aa, synthetic construct
SEVEFSYEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARNAKTGAAGSLMDVLHHPGMSHRVEITEGILADECAALLSDFFRMRRQGIKAQKKAQSSTD

SEQ ID NO: 37 - protein, 166 aa, synthetic construct
SEVEFSYEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARNAKTGAAGSLMDVLHHPGMSHRVEITEGILADECAALLSDFFRMRRQVIKAQKKAQSSTD

SEQ ID NO: 38 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGVRNAKTGAAGSLMDVLHHPGMNHRVEITEGILADECAALLSYFFRMRRQVIKAQKKAQSSTD

SEQ ID NO: 39 - protein, 166 aa, synthetic construct
AEVEFSHEYWMRHALTLAKRAWDEREVPVGAVLVHNNRVIGEGWNRPFGRHDPTAHAEIMALRQGGLVMQNYRLIDATLYVTLEPCVMCAGAMIHSRIGRVVFGVRNAKTGAAGSLMDVLHHPGMNHRVEITEGILADECAALLSYFFRMRRQVIKAQKKAQSSTD

SEQ ID NO: 40 - protein, 166 aa, synthetic construct
SEVEFSYEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGTRNAKTGAAGSLMDVLHHPGMSHRVEITEGILADECAALLSDFFRMRRQEIKAQSKAQSSTD

SEQ ID NO: 41 - protein, 166 aa, synthetic construct
SEVEFSHEYWMRHALTLAKRAWDEGEVPVGAVLVHNNRVIGEGWNRPIGRHDPTAHAEIMALRQGGLVMQNYRLIDATLYVTFEPCVMCAGAMIHSRIGRVVFGVHNAKTGAAGSLMDVLHYPGMNHRVEITEGILADECNDLLSYFFRMRRQVFKAQKKAQSSTD

SEQ ID NO: 42 - protein, 166 aa, synthetic construct
SEVEFSHEYWMRHALTLAKRAWDGGEVPVGAVLVHNNRVIGEGWNRPIGRHDPTAHAEIMALRQGGLVMQNYRLIDATLYVTFEPCVMCAGAMIHSRIGRVVFGVHNAKTGAAGSLMDVLHYPGMNHRVEITEGILADECNDLLSYFFRMRRQVFKAQKKAQSSTD

SEQ ID NO: 43 - protein, 166 aa, synthetic construct
SEVEFSHEYWMRHALTLAKRAWDDGEVPVGAVLVHNNRVIGEGWNRPIGRHDPTAHAEIMALRQGGLVMQNYRLIDATLYVTFEPCVMCAGAMIHSRIGRVVFGVKNAKTGAAGSLMDVLHYPGMNHRVEITEGILADECNGLLSYFFRMRRQVFKAQKKAQSSTD

SEQ ID NO: 44 - protein, 166 aa, synthetic construct
SEVEFSHEYWMRHALTLAKRAWDEQEVPVGAVLVHNNRVIGEGWNRPIGRHDPTAHAEIMALRQGGLVMQNYRLIDATLYVTFEPCVMCAGAMIHSRIGRVVFGVRNAKTGAAGSLMDVLHYPGMNHRVEITEGILADECNALLSYFFRMRRQVFKAQKKAQSSTD

SEQ ID NO: 45 - protein, 166 aa, synthetic construct
SEVEFSHEYWMRHALTLAKRAWDMGEVPVGAVLVHNNRVIGEGWNRPIGRHDPTAHAEIMALRQGGLVMQNYRLIDATLYVTFEPCVMCAGAMIHSRIGRVVFGVPNAKTGAAGSLMDVLHYPGMNHRVEITEGILADECNDLLSYFFRMRRQVFKAQKKAQSSTD

SEQ ID NO: 46 - protein, 166 aa, synthetic construct
SEVEFSHEYWMRHALTLAKRAWDECEVPVGAVLVHNNRVIGEGWNRPIGRHDPTAHAEIMALRQGGLVMQNYRLIDATLYVTFEPCVMCAGAMIHSRIGRVVFGVHNAKTGAAGSLMDVLHYPGMNHRVEITEGILADECNALLSYFFRMRRQVFKAQKKAQSSTD

SEQ ID NO: 47 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTFEPCVMCAGAMIHSRIGRVVFGVRNAKTGAAGSLMDVLHYPGMNHRVEITEGILADECNLLLSYFFRMRRQVFKAQKKAQSSTD

SEQ ID NO: 48 - protein, 166 aa, synthetic construct
SEVEFSHEYWMRHALTLAKRAWDEGEVPVGAVLVHNNRVIGEGWNRPIGRHDPTAHAEIMALRQGGLVMQNYRLIDATLYVTFEPCVMCAGAMIHSRIGRVVFGVRNAKTGAAGSLMDVLHYPGMNHRVEITEGILADECNALLSYFFRMRRQVFKAQKKAQSSTD

SEQ ID NO: 49 - protein, 166 aa, synthetic construct
SEVEFSHEYWMRHALTLAKRAWDEREVPVGAVLVHNNRVIGEGWNRPIGHHDPTAHAEIMALRQGGLVMQNYRLIDATLYVTFEPCVMCAGAMIHSRIGRVVFGVRNAKTGAAGSLMDVLHYPGMNHRVEITEGILADECAALLSYFFRMRRQVFNAQKKAQSSTD

SEQ ID NO: 50 - protein, 166 aa, synthetic construct
SEVEFSHEYWMRHALTLAKRAWDAGEVPVGAVLVHNNRVIGEGWNRPIGRHDPTAHAEIMALRQGGLVMQNYRLIDATLYVTFEPCVMCAGAMIHSRIGRVVFGVNNAKTGAAGSLMDVLHYPGMNHRVEITEGILADECNELLSYFFRMRRQVFKAQKKAQSSTD

SEQ ID NO: 51 - protein, 166 aa, synthetic construct
SEVEFSHEYWMRHALTLAKRAWDEREVPVGAVLVHTNRVIGEGWNRTIGRHDPTAHAEIMALRQGGLVMQNYRLIDATLYVTFEPCVMCAGAMIHSRIGRVVFGVRNAKTGAAGSLMDVLHYPGMNHRVEITEGILADECAALLSYFFRMRRQVFKAQKKAQSSTD

SEQ ID NO: 52 - protein, 166 aa, synthetic construct
SEVEFSHEYWMRHALTLAKRAWDEREVPVGAVLVHSNRVIGEGWNRPIGRHDPTAHAEIMALRQGGLVMQNYRLIDATLYVTFEPCVMCAGAMIHSRIGRVVFGVRNAKTGAAGSLMDVLHYPGMNHRVEITEGILADECAALLSYFFRMRRQVFKAQKKAQSSTD

SEQ ID NO: 53 - protein, 166 aa, synthetic construct
SEVEFSHEYWMRHALTLAKRAWDEREVPVGAVLVLNNRVIGEGWNRPIGRHDPTAHAEIMALRQGGLVMQNYRLIDATLYVTFEPCVMCAGAMIHSRIGRVVFGVRNAKTGAAGSLMDVLHYPGMNHRVEITEGILADECAALLSYFFRMRRQVFKAQKKAQSSTD

SEQ ID NO: 54 - protein, 166 aa, synthetic construct
SEVEFSHEYWMRHALTLAKRAWDEREVPVGAVLVLNNRVIGEGWNRLIGRHDPTAHAEIMALRQGGLVMQNYRLIDATLYVTFEPCVMCAGAMIHSRIGRVVFGVRNAKTGAAGSLMDVLHYPGMNHRVEITEGILADECAALLSYFFRMRRQVFKAQKKAQSSTD

SEQ ID NO: 55 - protein, 166 aa, synthetic construct
SEVEFSHEYWMRHALTLAKRAWDEREVPVGAVLVLNNRVIGEGWNRPIGRHDPTAHAEIMALRQGGLVMQNYRLIDATLYVTFEPCVMCAGAMIHSRIGRVVFGVRNAKTGAAGSLMDVLHYPGMNHRVEITEGILADECAALLSYFFRMRRQVFNAQKKAQSSTD

SEQ ID NO: 56 - protein, 166 aa, synthetic construct
SEVEFSHEYWMRHALTLAKRAWDEREVPVGAVLVLNNRVIGEGWNRPIGRHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRQVFKAQKKAQSSTD

SEQ ID NO: 57 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTFEPCVMCAGAMIHSRIGRVVFGVRNAKTGAAGSLMDVLHYPGMNHRVEITEGILADECAALLRYFFRMRRQVFKAQKKAQSSTD

SEQ ID NO: 58 - protein, 166 aa, synthetic construct
SEVEFSHEYWMRHALTLAKRAWDEREVPVGAVLVHSNRVIGEGWNRPIGHHDPTAHAEIMALRQGGLVMQNYRLIDATLYVTFEPCVMCAGAMIHSRIGRVVFGVRNAKTGAAGSLMDVLHYPGMNHRVEITEGILADECAALLSYFFRMRRQVFKAQKKAQSSTD

SEQ ID NO: 59 - protein, 166 aa, synthetic construct
SEVEFSHEYWMRHALTLAKRAWDEREVPVGAVLVHNNRVIGEGWNRPIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLSYFFRMRRQVFNAQKKAQSSTD

SEQ ID NO: 60 - protein, 166 aa, synthetic construct
SEVEFSHEYWMRHALTLAKRAWDEREVPVGAVLVHNNRVIGEGWNRSIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 61 - protein, 166 aa, synthetic construct
SEVEFSHEYWMRHALTLAKRAWDEREVPVGAVLVHNNRVIGEGWNRTIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 62 - protein, 166 aa, synthetic construct
SEVEFSHEYWMRHALTLAKRAWDEREVPVGAVLVHNNRVIGEGWNRAIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 63 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECNALLSDFFRMRRQEIKAQKKAQSSTD

SEQ ID NO: 64 - protein, 166 aa, synthetic construct
SEVEFSHEYWMRHALTLAKRARDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 65 - protein, 166 aa, synthetic construct
SEVEFSHEYWMRHALTLAKRALDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 66 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMPRQEIKAQKKAQSSTD

SEQ ID NO: 67 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMHRQEIKAQKKAQSSTD

SEQ ID NO: 68 - protein, 166 aa, synthetic construct
SEVEFSHEYWMRHALTLAKRAWDEREVPVGAVLVHNNRVIGEGWNRPIGRHDPTAHAEIMALRQGGLVMQNYRLIDATLYVTFEPCVMCAGAMIHSRIGRVVFGVRNAKTGAAGSLMDVLHYPGMNHRVEITEGILADECAALLSYFFRMRRQVFKAQKKAQSSTD

SEQ ID NO: 69 - protein, 166 aa, synthetic construct
SEVEFSHEYWMRHALTLAKRAWDEREVPVGAVLVLNNRVIGEGWNRPIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRQVFNAQKKAQSSTD

SEQ ID NO: 70 - protein, 166 aa, synthetic construct
SEVEFSHEYWMRHALTLAKRAWDEREVPVGAVLVLNNRVIGEGWNRSIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRQVFNAQKKAQSSTD

SEQ ID NO: 71 - protein, 166 aa, synthetic construct
SEVEFSHEYWMRHALTLAKRAWDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRQVFNAQKKAQSSTD

SEQ ID NO: 72 - protein, 166 aa, synthetic construct
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 73 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 74 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNAKTGAAGSLMDVLHYPGMNHRVEITEGILADECAALLCYFFRMPRQVFNAQKKAQSSTD

SEQ ID NO: 75 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 76 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSIN

SEQ ID NO: 77 - protein, 7 aa, synthetic construct
PKKKRKV

SEQ ID NO: 78 - protein, 19 aa, synthetic construct
MKRTADGSEFESPKKKRKV

SEQ ID NO: 79 - protein, 30 aa, synthetic construct
MDSLLMNRRKFLYQFKNVRWAKGRRETYLC

SEQ ID NO: 80 - protein, 20 aa, synthetic construct
AVKRPAATKKAGQAKKKKLD

SEQ ID NO: 81 - protein, 25 aa, synthetic construct
MSRRRKANPTKLSENAKKLAKEVEN

SEQ ID NO: 82 - protein, 9 aa, synthetic construct
PAAKRVKLD

SEQ ID NO: 83 - protein, 9 aa, synthetic construct
KLKIKRPVK

SEQ ID NO: 84 - protein, 8 aa, synthetic construct
VSRKRPRP

SEQ ID NO: 85 - protein, 10 aa, synthetic construct
EGAPPAKRAR

SEQ ID NO: 86 - protein, 12 aa, synthetic construct
PPQPKKKPLDGE

SEQ ID NO: 87 - protein, 17 aa, synthetic construct
KRTADGSEFEPKKKRKV

SEQ ID NO: 88 - protein, 18 aa, synthetic construct
KRTADGSEFESPKKKRKV

SEQ ID NO: 89 - protein, 150 aa, synthetic construct
  VARIANT 6..150: note = may be absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 90 - protein, 30 aa, synthetic construct
  VARIANT 2..30: note = may be absent
GGGGGGGGGGGGGGGGGGGGGGGGGGGGGG

SEQ ID NO: 91 - protein, 150 aa, synthetic construct
  VARIANT 6..150: note = may be absent
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 92 - protein, 90 aa, synthetic construct
  VARIANT 4..90: note = may be absent
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 93 - protein, 120 aa, synthetic construct
  VARIANT 5..120: note = may be absent
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 94 - protein, 60 aa, synthetic construct
  VARIANT 3..60: note = may be absent
XPXPXPXPXPXPXPXPXPXPXPXPXPXPXPXPXPXPXPXPXPXPXPXPXPXPXPXPXPXP

SEQ ID NO: 95 - protein, 16 aa, synthetic construct
SGSETPGTSESATPES

SEQ ID NO: 96 - protein, 12 aa, synthetic construct
SGSETPGTSESA

SEQ ID NO: 97 - protein, 21 aa, synthetic construct
SGSETPGTSESATPEGGSGGS

SEQ ID NO: 98 - protein, 32 aa, synthetic construct
SGGSSGGSSGSETPGTSESATPESSGGSSGGS

SEQ ID NO: 99 - protein, 10 aa, synthetic construct
SGGSGGSGGS

SEQ ID NO: 100 - protein, 4 aa, synthetic construct
SGGS

SEQ ID NO: 101 - protein, 60 aa, synthetic construct
SGGSSGGSSGSETPGTSESATPESAGSYPYDVPDYAGSAAPAAKKKKLDGSGSGGSSGGS

SEQ ID NO: 102 - protein, 6 aa, synthetic construct
GGSGGS

SEQ ID NO: 103 - protein, 9 aa, synthetic construct
GGSGGSGGS

SEQ ID NO: 104 - protein, 33 aa, synthetic construct
SGGSSGGSSGSETPGTSESATPESSGGSSGGSS

SEQ ID NO: 105 - protein, 1775 aa, synthetic construct
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSPKKKRKV

SEQ ID NO: 106 - protein, 1605 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKV

SEQ ID NO: 107 - protein, 1605 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKV

SEQ ID NO: 108 - DNA, 4916 bp, synthetic construct
  source 1..4916: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagatcagggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgaaacggacagccgacggaagcgagttcgagtcaccaaagaagaagcggaaagtctctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcctgtcctacgagacagagatcctgacagtggagtatggcctgctgccaatcggcaagatcgtggagaagaggatcgagtgtaccgtgtactctgtggataacaatggcaacatctatacacagcccgtggcacagtggcacgataggggagagcaggaggtgttcgagtattgcctggaggacggcagcctgatcagggcaaccaaggaccacaagttcatgacagtggatggccagatgctgcccatcgacgagattttcgagcgggagctggacctgatgagagtggataacctgcctaatagcggaggcagtaaaagaacagcagacgggagtgagtttgagcccaagaaaaagagaaaggtgtaagatctgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 109 - DNA, 4322 bp, synthetic construct
  source 1..4322: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagatcagggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggctggaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcctgtcctacgagacagagatcctgacagtggagtatggcctgctgccaatcggcaagatcgtggagaagaggatcgagtgtaccgtgtactctgtggataacaatggcaacatctatacacagcccgtggcacagtggcacgataggggagagcaggaggtgttcgagtattgcctggaggacggcagcctgatcagggcaaccaaggaccacaagttcatgacagtggatggccagatgctgcccatcgacgagattttcgagcgggagctggacctgatgagagtggataacctgcctaatagcggaggcagtaaaagaacagcagacgggagtgagtttgagcccaagaaaaagagaaaggtgtaagatctgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 110 - DNA, 4570 bp, synthetic construct
  source 1..4570: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctctagatcagggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatgaaacggacagccgacggaagcgagttcgagtcaccaaagaagaagcggaaagtcatcaagattgctacacggaaatacctgggaaagcagaacgtgtacgacatcggcgtggagcgggatcacaacttcgccctgaagaatggctttatcgccagcaattgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtctaagatctgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagaaaaaaagcaccgactcggtgccactttttcaagttgataacggactagccttattttaacttgctatttctagctctaaaactctgtgatccgtaagtgatacggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaccttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 111 - protein, 10 aa, synthetic construct
TPSVLRQXYN

SEQ ID NO: 112 - DNA, 30 bp, synthetic construct
  source 1..30: mol_type = other DNA
actccgtctgtgctccgtcagtgatacaac

SEQ ID NO: 113 - DNA, 30 bp, synthetic construct
  source 1..30: mol_type = other DNA
gttgtatcactgacggagcacagacggagt

SEQ ID NO: 114 - protein, 10 aa, synthetic construct
TPSVIRKXYN

SEQ ID NO: 115 - DNA, 30 bp, synthetic construct
  source 1..30: mol_type = other DNA
accccctctgtgatccgtaagtgatacaac

SEQ ID NO: 116 - DNA, 30 bp, synthetic construct
  source 1..30: mol_type = other DNA
gttgtatcacttacggatcacagagggggt

(19) United States
(12) Patent Application Publication
(10) Pub. No.: US 2026/0357298 A1
(43) Pub. Date: Nov. 12, 2026

(54) METHODS AND COMPOSITIONS FOR BASE EDITING OF TPP1 IN THE TREATMENT OF BATTEN DISEASE

(71) Applicants: The Broad Institute, Inc., Cambridge, MA (US); President and Fellows of Harvard College, Cambridge, MA (US); Sanford Health, Sioux Falls, SD (US)

(72) Inventors: David R. Liu, Cambridge, MA (US); Peyton Barksdale Randolph, Cambridge, MA (US); Jill Weimer, Sioux Falls, SD (US); Gregory Newby, Cambridge, MA (US)

(73) Assignees: The Broad Institute, Inc., Cambridge, MA (US); President and Fellows of Harvard College, Cambridge, MA (US); Sanford Health, Sioux Falls, SD (US)

(21) Appl. No.: 19/699,243

(22) Filed: Jun. 5, 2026

Related U.S. Application Data
(63) Continuation of application No. PCT/US2024/058638, filed on Dec. 5, 2024.
(60) Provisional application No. 63/606,808, filed on Dec. 6, 2023.

(51) Int. Cl. C12N 15/90; A61P 25/28; C12N 9/22; C12N 9/78; C12N 15/11; C12N 15/86

(52) U.S. Cl. CPC C12N 15/907; A61P 25/28; C12N 9/226; C12N 9/78; C12N 15/11; C12N 15/86; C12N 2310/20; C12N 2750/14143

(57) ABSTRACT

The present disclosure provides methods of editing Tpp1 using a base editor (e.g., for correcting an R208X mutation in a Tpp1 enzyme, where X is a premature stop codon) and a gRNA. Such methods may be useful for treating Batten disease. The present disclosure also provides gRNAs and base editor-gRNA complexes for editing Tpp1 and treating Batten disease. Polynucleotides, vectors, AAV particles, cells, and kits for editing Tpp1 and treating Batten disease are also provided herein.

### RELATED APPLICATIONS

This application claims priority under 35 U.S.C. §§ 120 and 365(c) to International PCT Application, PCT/US2024/058638, filed on Dec. 5, 2024, which claims priority under 35 U.S.C. § 119(e) to U.S. Provisional Application, U.S. Ser. No. 63/606,808, filed Dec. 6, 2023, which is incorporated herein by reference.

### FEDERALLY SPONSORED RESEARCH

This invention was made with government support under Grant Nos. AI142756, GM118062, HG009490, HL156647, NS132304, and NS132315 awarded by the National Institutes of Health. The government has certain rights in the invention.

### REFERENCE TO AN ELECTRONIC SEQUENCE LISTING

The contents of the electronic sequence listing (B119570193US01-SEQ-GJM.xml; Size: 170,496 bytes; and Date of Creation: May 29, 2026) are herein incorporated by reference in their entirety.

### BACKGROUND OF THE INVENTION

Batten disease is a nervous system disorder that typically begins with onset of symptoms during childhood, generally within two to four years of age. Mutation of R208 in the human tripeptidyl-peptidase 1 (Tpp1) gene causes premature termination and dysfunction of the Tppl enzyme. This leads to gradual neural degeneration presenting as ataxia, epilepsy, and blindness. The R208X mutation (where X is a premature stop codon) in the Tppl enzyme is caused by a C·G-to-T·A transition mutation in Tpp1. Accordingly, a means to reverse this transition mutation and treat Batten disease is needed.

### SUMMARY OF THE INVENTION

The present disclosure describes methods, uses, compositions, and systems that utilize adenosine base editors and guide RNAs to treat Batten disease. Editing strategies leading to reversal of the pathogenic mutation R208X in the human Tpp1 gene (and the analogous R207X mutation in the mouse Tpp1 gene) have been developed as described herein.

Thus, in one aspect, the present disclosure provides methods of base editing a tripeptidyl-peptidase 1 (Tpp1) gene comprising contacting a nucleic acid sequence encoding the Tpp1 gene with a base editor and a guide RNA (gRNA), which targets the base editor to the Tpp1 gene. In some embodiments, the gRNA targets a protospacer in the Tpp1 gene comprising a nucleotide sequence at least 80%, at least 85%, at least 90%, at least 95%, at least 96%, at least 97%, at least 98%, or at least 99% identical to the sequence TATCACTGACGGAGCACAGA (SEQ ID NO: 1), TATCACTTACGGATCACAGA (SEQ ID NO: 2), or a fragment thereof, or a sequence comprising one, two, three, four, or five mutations relative to TATCACTGACGGAGCACAGA (SEQ ID NO: 1), TATCACTTACGGATCACAGA (SEQ ID NO: 2), or a fragment thereof, or a sequence shifted 1, 2, 3, 4, 5, 6, 7, 8, 9, or 10 nucleotides upstream or downstream relative to SEQ ID NO: 1 or 2 in a Tpp1 gene of SEQ ID NO: 8 or 10. In some embodiments, the gRNA comprises a spacer at least 80%, at least 85%, at least 90%, at least 95%, at least 96%, at least 97%, at least 98%, or at least 99% identical to the sequence GTATCACTGACGGAGCACAGA (SEQ ID NO: 3) or GTATCACTTACGGATCACAGA (SEQ ID NO: 4), or a sequence comprising one, two, three, four, or five mutations relative to GTATCACTGACGGAGCACAGA (SEQ ID NO: 3) or GTATCACTTACGGATCACAGA (SEQ ID NO: 4). In some embodiments, polynucleotides encoding the base editor and gRNA are delivered to the nucleic acid sequence encoding Tpp1 (for example, a nucleic acid sequence in a cell), e.g., in one or more AAV particles.

In some embodiments, the methods result in correction of a C·G-to-T·A transition mutation in the Tpp1 gene. In some embodiments, correction of the C·G-to-T·A transition mutation in the Tpp1 gene results in correction of an R208X mutation in a human Tpp l protein of SEQ ID NO: 9 or an R207X mutation in a mouse Tppl protein of SEQ ID NO: 11, where X is a premature stop codon. Analogous positions in the Tpp1 proteins of other species may also be targeted for correction. In certain embodiments, the method is a method of treating Batten disease in a subject.

In another aspect, the present disclosure provides gRNAs targeting a protospacer in a Tpp1 gene comprising a nucleotide sequence at least 80%, at least 85%, at least 90%, at least 95%, at least 96%, at least 97%, at least 98%, or at least 99% identical to the sequence TATCACTGACGGAGCACAGA (SEQ ID NO: 1), TATCACTTACGGATCACAGA (SEQ ID NO: 2), or a fragment thereof, or a sequence comprising one, two, three, four, or five mutations relative to TATCACTGACGGAGCACAGA (SEQ ID NO: 1), TATCACTTACGGATCACAGA (SEQ ID NO: 2), or a fragment thereof, or a sequence comprising one, two, three, four, or five mutations relative to TATCACTGACGGAGCACAGA (SEQ ID NO: 1), TATCACTTACGGATCACAGA (SEQ ID NO: 2), or a fragment thereof, or a sequence shifted 1, 2, 3, 4, 5, 6, 7, 8, 9, or 10 nucleotides upstream or downstream relative to SEQ ID NO: 1 or 2 in a Tpp1 gene of SEQ ID NO: 8 or 10. In some embodiments, the gRNAs comprise a spacer at least 80%, at least 85%, at least 90%, at least 95%, at least 96%, at least 97%, at least 98%, or at least 99% identical to the sequence GTATCACTTACGGATCACAGA (SEQ ID NO: 4) or GTATCACTGACGGAGCACAGA (SEQ ID NO: 3), or a sequence comprising one, two, three, four, or five mutations relative to GTATCACTTACGGATCACAGA (SEQ ID NO: 4) or GTATCACTGACGGAGCACAGA (SEQ ID NO: 3). In certain embodiments, the gRNAs comprise a sequence at least 80%, at least 85%, at least 90%, at least 95%, at least 96%, at least 97%, at least 98%, or at least 99% identical to the sequence

(SEQ ID NO: 7)
GTATCACTTACGGATCACAGAGTTTTAGAGCTAGAAATAG
CAAGTTAAAATAAG
(SEQ ID NO: 6)
GCTAGTCCGTTATCAACTTGAAAAAGTGGCACCGAGTCGGTGC or
GTATCACTGACGGAGCACAGAGTTTTAGAGCTAGAAATAGCAAGTT
AAAATAAGGCTAGTCCGTTATCAACTTGAAAAAGTGGCACCGAGTC
GGTGC

In another aspect, the present disclosure provides complexes comprising any of the gRNAs provided herein and a base editor. In some embodiments, the base editor is an adenosine base editor. In some embodiments, the base editor comprises ABE7.10, ABE8e, ABE8e(V106W), or a variant thereof.

In another aspect, the present disclosure provides one or more AAV particles (e.g., using a split intein base editor approach) comprising one or more polynucleotides encoding any of the gRNAs and base editors provided herein.

In another aspect, the present disclosure provides one or more polynucleotides encoding any of the gRNAs provided herein. In another aspect, the present disclosure provides polynucleotides encoding any of the gRNAs and base editors of the complexes provided herein.

In another aspect, the present disclosure provides vectors comprising any of the polynucleotides provided herein.

In another aspect, the present disclosure provides pharmaceutical compositions comprising any of the gRNAs, complexes, AAV particles, polynucleotides, or vectors provided herein.

In another aspect, the present disclosure provides cells comprising any of the gRNAs, complexes, AAV particles, polynucleotides, or vectors provided herein.

In another aspect, the present disclosure provides kits comprising any of the gRNAs, complexes, AAV particles, polynucleotides, or vectors provided herein.

In another aspect, the present disclosure provides for the use of any of the gRNAs, complexes, AAV particles, polynucleotides, vectors, or pharmaceutical compositions provided herein in the manufacture of a medicament for the treatment of a disease (e.g., Batten disease).

In another aspect, the present disclosure provides for the use of any of the gRNAs, complexes, AAV particles, polynucleotides, vectors, or pharmaceutical compositions provided herein in medicine.

The foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 shows in vitro correction of mouse Tpp1 R207X with adenosine base editors ABE7.10 and ABE8eV106W.

FIG. 2 shows an in vitro activity assay for edited and non-edited Tpp1.

FIG. 3 shows in vivo correction of mouse Tpp1 R207X with ABE7.10-SpCas9.

FIG. 4 shows an in vivo RNAscope assay demonstrating AAV delivery of a base editor-gRNA system for Tpp1 editing.

FIGS. 5A-5B show in vivo adenine base editing of Tpp1. FIG. 5A shows ABE7.10 Tpp1 editing levels in bulk brain tissue of mice. FIG. 5B shows levels of silent and non-silent bystander mutations introduced during Tpp1 editing.

FIG. 6 shows in vivo enzyme activity of Tppl protein in tissues isolated from treated mice.

FIG. 7 shows an in vivo assay of ATP synthase subunit C (SubC) levels in edited and non-edited tissues. SubC is a biomarker of degeneration resulting from Tpp1 mutation.

FIG. 8 shows an in vivo assay of microgliosis (CD68 expression levels) in edited and non-edited tissues. CD68 expression level is a biomarker of degeneration resulting from Tpp1 mutation.

FIG. 9 shows an in vivo assay of astrocytosis (GFAP expression levels) in edited and non-edited tissues. GFAP expression level is another biomarker of degeneration resulting from Tpp1 mutation.

FIGS. 10A-10C show evaluation of adenine base editors for the correction of Tpp1 R207X. FIG. 10A provides a schematic of the target locus and encoded amino acid sequence in Cln2^{R207X-/-} mice (top) and humans (bottom). The evaluated ABE protospacer sequences targeted by SpCas9 and SaCas9 are underlined with the respective PAM. Adenines targeted by these protospacers are numbered according to their position in the SpCas9 protospacer, numbered from the 5′ end. Editing of the highlighted adenine in position 5 reverts the pathogenic nonsense mutation to a wild-type Arg codon, whereas editing of other adenines in the indicated protospacers would cause non-silent bystander mutations. FIG. 10B shows the percent editing efficiency at Tpp1 measured by high-throughput sequencing of gDNA from Cln2^{R20X-/-} mouse embryonic fibroblasts (MEFs) 48 hours post electroporation with the specified ABE mRNA and an sgRNA targeting the corresponding protospacer. Editing data are shown for positions where mean editing efficiency was ≥1.0% in any condition. SpCas9-ABE7.10 mRNA and a non-targeting sgRNA were electroporated for the non-targeting condition. FIG. 10C shows allele frequencies of the ABE-treated Cln2^{R207X-/-} MEF gDNA in FIG. 10B. Data are presented as mean±s.d. of n=3 independent biological replicates.

FIGS. 11A-11B show characterization of TPP1 activity following adenine base editing. FIG. 11A shows tripeptidyl peptidase 1 (TPP1) enzyme activity of the major allele products generated by targeting Tpp1 R207X with adenine base editors (ABEs). FIG. 11B shows TPP1 activity in C1n2^{R207X-/-} mouse embryonic fibroblasts (MEFs) 48 hours post electroporation with the specified ABE mRNA and an sgRNA targeting Tpp1 R207X. Data is normalized to TPP1 activity in WT MEFs electroporated with a non-targeting ABE. SpCas9-ABE7.10 mRNA and a non-targeting sgRNA were electroporated for the non-targeted conditions. Data are presented as mean±s.d. of n=3 independent biological replicates. Statistical significance was calculated by one-way ANOVA; *p<0.05, ****p<0.0001.

FIGS. 12A-12D show efficient viral transduction and adenine base editing from a single injection of dual-AAV9 ABEs in Cln2^{R207X-/-} mice. FIG. 12A provides a schematic of dual-vector AAV9.SpCas9-ABE7.10 architecture for correction of Tpp1 R207X. FIG. 12B shows co-transduction efficiencies for AAV9.SpCas9-ABE7.10 and AAV9.SpCas9-ABE8eV106W in the cortex, hippocampus and thalamus 11 weeks after a single ICV injection of 5×10¹⁰ vg (2.5×10¹⁰ vg each intein half) into P1 Cln2^{R207X-/-} mice. Transduction efficiencies are reported as mean±s.e.m. percentages of DAPI⁺ cells expressing both vector transgenes out of total DAPI⁺ cells. FIG. 12C shows bulk cortical gDNA editing efficiency measured by high-throughput sequencing of Tpp1 R207X. FIG. 12D shows allele frequencies of the ABE-treated Cln2^{R207X-/-} cortical gDNA in FIG. 12C. Data in FIG. 12C and FIG. 12D are presented as mean±s.d. of n=12 mice.

FIG. 13 shows TPP1 enzyme activity after AA9.SpCas9-ABE7.10 treatment. TPP1 activity from bulk cortical lysates 11 weeks after a P1 intracerebroventricular (ICV) injection of AAV9.SpCas9-ABE7.10 (5×10¹⁰ vg total, 2.5×10¹⁰ vg each intein half) or PBS. Data is normalized to TPP1 activity in wild-type mice injected ICV with PBS. Statistical significance was calculated by one-way ANOVA; ****p<0.0001.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

### Adenosine Deaminase

As used herein, the term “adenosine deaminase” or “adenosine deaminase domain” refers to a protein or enzyme that catalyzes a deamination reaction of an adenosine (or adenine). The terms are used interchangeably. In certain embodiments, the disclosure provides nucleobase editor fusion proteins comprising one or more adenosine deaminase domains (e.g., fused to a napDNAbp such as a Cas9 protein). For instance, an adenosine deaminase domain may comprise a heterodimer of a first adenosine deaminase and a second deaminase domain, connected by a linker. Adenosine deaminases (e.g., engineered adenosine deaminases or evolved adenosine deaminases) provided herein may be enzymes that convert adenine (A) to inosine (I) in DNA or RNA. Such adenosine deaminases can lead to an A:T to G:C base pair conversion. In some embodiments, the deaminase is a variant of a naturally-occurring deaminase from an organism (e.g., bacteria, such as E. coli). In some embodiments, the deaminase does not occur in nature. For example, in some embodiments, the deaminase is at least 80%, at least 85%, at least 90%, at least 95%, at least 96%, at least 97%, at least 98%, or at least 99% identical to a naturally-occurring deaminase.

In some embodiments, the adenosine deaminase is derived from a bacterium, such as, E. coli, S. aureus, S. typhi, S. putrefaciens, H. influenzae, C. jejuni, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the truncated ecTadA may be missing 1, 2, 3, 4, 5, 6, 7, 8, 9, 10, 11, 12, 13, 14, 15, 6, 17, 18, 19, or 20 C-terminal amino acid residues relative to the full length ecTadA. In some embodiments, the ecTadA deaminase does not comprise an N-terminal methionine. In some embodiments, the adenosine deaminase comprises ecTadA(8e) (i.e., as used in the base editor ABE8e) as described further herein. Adenosine deaminases are further described, for example, in International Patent Application Publication No. WO 2018/027078, which is incorporated herein by reference.

### Base Editing

“Base editing” refers to genome editing technology that involves the conversion of a specific nucleic acid base into another at a targeted genomic locus. In certain embodiments, this can be achieved without requiring double-stranded DNA breaks (DSB), or single stranded breaks (i.e., nicking). Many other genome editing techniques, including CRISPR-based systems, begin with the introduction of a DSB at a locus of interest. Subsequently, cellular DNA repair enzymes mend the break, commonly resulting in random insertions or deletions (indels) of bases at the site of the DSB. However, when the introduction or correction of a point mutation at a target locus is desired rather than stochastic disruption of the entire gene, these genome editing techniques are unsuitable, as correction rates are low (e.g., typically 0.1% to 5%), with the major genome editing products being indels. In order to increase the efficiency of gene correction without simultaneously introducing random indels, the CRISPR system is modified to directly convert one DNA base into another without DSB formation. See, Komor, A. C., et al., Programmable editing of a target base in genomic DNA without double-stranded DNA cleavage. Nature 533, 420-424 (2016), the entire contents of which is incorporated by reference herein. In some embodiments, base editing is accomplished using a fusion protein comprising a deaminase and napDNAbp (e.g., a Cas9 protein).

In principle, there are 12 possible base-to-base changes that may occur via individual or sequential use of transition (i.e., a purine-to-purine change or pyrimidine-to-pyrimidine change) or transversion (i.e., a purine-to-pyrimidine or pyrimidine-to-purine) editors. These include transition base editors such as the cytosine base editor (“CBE”), also known as a C-to-T base editor (or “CTBE”). This type of editor converts a C:G Watson-Crick nucleobase pair to a T:A Watson-Crick nucleobase pair. Because the corresponding Watson-Crick paired bases are also interchanged as a result of the conversion, this category of base editor may also be referred to as a guanine base editor (“GBE”) or G-to-A base editor (or “GABE”). Other transition base editors include the adenine base editor (or “ABE”), also known as an A-to-G base editor (“AGBE”). This type of editor converts an A:T Watson-Crick nucleobase pair to a G:C Watson-Crick nucleobase pair. Because the corresponding Watson-Crick paired bases are also interchanged as a result of the conversion, this category of base editor may also be referred to as a thymine base editor (or “TBE”) or T-to-G base editor (“TGBE”).

### Base Editors

The terms “base editor (BE)” and “nucleobase editor,” which are used interchangeably herein, refer to an agent comprising a polypeptide that is capable of making a modification to a base (e.g., A, T, C, G, or U) within a nucleic acid sequence (e.g., DNA or RNA) that converts one base to another (e.g., A to G, A to C, A to T, C to T, C to G, C to A, G to A, G to C, G to T, T to A, T to C, or T to G). In some embodiments, the base editor is capable of deaminating a base within a nucleic acid, such as a base within a DNA molecule. In some embodiments, a base editor is an adenosine base editor. In the case of an adenosine base editor, the base editor is capable of deaminating an adenine (A) in DNA. Such base editors may include a nucleic acid programmable DNA binding protein (napDNAbp) fused to an adenosine deaminase. Some base editors include CRISPR-mediated fusion proteins that are utilized in the base editing methods described herein. In some embodiments, the base editor comprises a Cas9 protein fused to a deaminase that binds a nucleic acid in a guide RNA-programmed manner via the formation of an R-loop, but does not cleave the nucleic acid.

In some embodiments, a base editor is a macromolecule or macromolecular complex that results primarily (e.g., more than 80%, more than 85%, more than 90%, more than 95%, more than 99%, more than 99.9%, or 100%) in the conversion of a nucleobase in a polynucleotide sequence into another nucleobase (i.e., a transition or transversion) using a combination of 1) a nucleotide-, nucleoside-, or nucleobase-modifying enzyme, and 2) a nucleic acid binding protein that can be programmed to bind to a specific nucleic acid sequence.

In some embodiments, the base editor comprises a DNA binding domain (e.g., a programmable DNA binding domain, such as a Cas9 protein) that directs it to a target sequence. In some embodiments, the base editor comprises a nucleobase modification domain fused to a programmable DNA binding domain (e.g., a Cas9 protein). The terms “nucleobase modifying enzyme” and “nucleobase modification domain,” which are used interchangeably herein, refer to an enzyme that can modify a nucleobase and convert one nucleobase to another (e.g., a deaminase, such as a cytidine deaminase or an adenosine deaminase). In some embodiments, A to G editing is carried out by a deaminase, e.g., an adenosine deaminase.

In some embodiments, a base editor converts an A to a G. In some embodiments, the base editor comprises an adenosine deaminase. An “adenosine deaminase” is an enzyme involved in purine metabolism. It is needed for the breakdown of adenosine from food and for the turnover of nucleic acids in tissues. Its primary function in humans is the development and maintenance of the immune system. An adenosine deaminase catalyzes hydrolytic deamination of adenosine (forming inosine, which base pairs as G) in the context of DNA. There are no known natural adenosine deaminases that act on DNA. Instead, known adenosine deaminase enzymes only act on RNA (tRNA or mRNA). Evolved deoxyadenosine deaminase enzymes that accept DNA substrates and deaminate dA to deoxyinosine have been described, e.g., in International Patent Application No. PCT/US2017/045381, filed Aug. 3, 2017, which published as WO 2018/027078, International Patent Application No. PCT/US2019/033848, which published as WO 2019/226953, International Patent Application No PCT/US2019/033848, filed May 23, 2019, which published as WO 2019226953, and International Patent Application No. PCT/US2020/028568, filed Apr. 17, 2020, which published as WO 2020214842; each of which is incorporated herein by reference.

Exemplary adenosine and cytidine nucleobase editors are also described in Rees & Liu, “Base editing: precision chemistry on the genome and transcriptome of living cells,” Nat. Rev. Genet. 2018; 19(12):770-788; as well as U.S. Patent Application Publication No. 2018/0073012, published Mar. 15, 2018, which issued as U.S. Pat. No. 10,113,163 on Oct. 30, 2018; U.S. Patent Application Publication No. 2017/0121693, published May 4, 2017, which issued as U.S. Pat. No. 10,167,457 on Jan. 1, 2019; PCT Application Publication No. WO 2017/070633, published Apr. 27, 2017; U.S. Patent Application Publication No. 2015/0166980, published Jun. 18, 2015; U.S. Pat. No. 9,840,699, issued Dec. 12, 2017; and U.S. Pat. No. 10,077,453, issued Sep. 18, 2018, each of which is incorporated herein by reference.

### Batten Disease

The term “Batten disease” refers a group of nervous system disorders known as neuronal ceroid lipofuscinoses. Late infantile neuronal ceroid lipofuscinosis type 2 (CLN2) is a rare and rapidly progressing form of Batten disease. Specifically, CLN2 is a pediatric brain disorder and one of the most common forms of neuronal ceroid lipofuscinosis. Onset of symptoms of Batten disease typically begins during childhood (e.g., in children under ten years of age, and often within two to four years of age). Batten disease is caused by the mutation R208X (where X is a premature stop codon) in the human tripeptidyl-peptidase 1 (Tpp1) gene. The R208X mutation results in premature termination and dysfunction of the Tpp1 enzyme, leading to gradual neural degeneration. Symptoms typically present as ataxia, epilepsy, and blindness. The R208X mutation in the Tpp1 enzyme is caused by a C·G-to-T·A transition mutation in Tpp1.

### Cas9

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain,” as used herein, is a protein fragment comprising an active or fully or partly inactive cleavage domain of Cas9 and/or the gRNA binding domain of Cas9. A “Cas9 protein” is a full length Cas9 protein. A Cas9 nuclease is also referred to sometimes as a casn1 nuclease or a CRISPR (Clustered Regularly Interspaced Short Palindromic Repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (rnc), and a Cas9 domain. The tracrRNA serves as a guide for ribonuclease 3-aided processing of pre-crRNA. Subsequently, Cas9/crRNA/tracrRNA endonucleolytically cleaves a linear or circular dsDNA target complementary to the spacer. The strand in the target DNA not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the contents of which are incorporated herein by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease comprises one or more mutations that partially impair or inactivate the DNA cleavage domain.

A nuclease-inactivated Cas9 domain may interchangeably be referred to as a “dCas9” protein (for nuclease-“dead” Cas9). Methods for generating a Cas9 domain (or a fragment thereof) having an inactive DNA cleavage domain are known (see, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)). In some embodiments, a Cas9 protein comprises one or more mutations to inactivate the nuclease activity of only one of the HNH subdomain or the RuvC1 subdomain.

In some embodiments, proteins comprising fragments of a Cas9 protein are provided. For example, in some embodiments, a protein comprises one of two Cas9 domains: (1) the gRNA binding domain of Cas9; or (2) the DNA cleavage domain of Cas9. In some embodiments, proteins comprising Cas9, or fragments thereof, are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, at least about 99.8% identical, or at least about 99.9% identical to wild type Cas9 (e.g., SpCas9 of SEQ ID NO: 12). In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9 (e.g., SpCas9 of SEQ ID NO: 12). In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9 (e.g., SpCas9 of SEQ ID NO: 12). In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 (e.g., SpCas9 of SEQ ID NO: 12).

### Deaminase

The term “deaminase” or “deaminase domain” refers to a protein or enzyme that catalyzes a deamination reaction. In some embodiments, the deaminase is an adenosine (or adenine) deaminase, which catalyzes the hydrolytic deamination of adenine or adenosine. In some embodiments, the adenosine deaminase catalyzes the hydrolytic deamination of adenine or adenosine in deoxyribonucleic acid (DNA) to inosine.

The deaminases provided herein may be from any organism, such as a bacterium. In some embodiments, the deaminase or deaminase domain is a variant of a naturally occurring deaminase from an organism. In some embodiments, the deaminase or deaminase domain does not occur in nature. For example, in some embodiments, the deaminase or deaminase domain is at least 80%, at least 85%, at least 90%, at least 95%, at least 96%, at least 97%, at least 98%, or at least 99% identical to a naturally occurring deaminase.

### Fusion Protein

The term “fusion protein” as used herein refers to a hybrid polypeptide that comprises protein domains from at least two different proteins. One protein may be located at the amino-terminal (N-terminal) portion of the fusion protein or at the carboxy-terminal (C-terminal) protein, thus forming an “amino-terminal fusion protein” or a “carboxy-terminal fusion protein,” respectively. A protein may comprise different domains, for example, a Cas9 protein fused to a deaminase (i.e., a base editor). Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which is incorporated herein by reference.

Guide RNA (“gRNA”)

As used herein, the term “guide RNA” is a particular type of guide nucleic acid which is commonly associated with a Cas protein (e.g., a Cas9 protein), directing the Cas protein to a specific sequence in a DNA molecule that includes complementarity to the protospacer sequence of the guide RNA. For example, a gRNA may direct a Cas protein (e.g., as part of a base editor) to a target site in the Tpp1 gene. However, this term also embraces the equivalent guide nucleic acid molecules that associate with Cas protein equivalents, homologs, orthologs, or paralogs, whether naturally occurring or non-naturally occurring (e.g., engineered or recombinant), and which otherwise program the Cas protein equivalent to localize to a specific target nucleotide sequence. The Cas protein equivalents may include other napDNAbps from any type of CRISPR system (e.g., type II, V, VI), including Cpf1 (a type-V CRISPR-Cas system), C2cl (a type V CRISPR-Cas system), C2c2 (a type VI CRISPR-Cas system), and C2c3 (a type V CRISPR-Cas system). Further Cas-equivalents are described in Makarova et al., “C2c2 is a single-component programmable RNA-guided RNA-targeting CRISPR effector,” Science 2016; 353(6299), which is incorporated herein by reference. Exemplary sequences and structures of guide RNAs are provided herein.

Functionally, guide RNAs associate with a Cas protein, directing (or programming) the Cas protein to a specific sequence in a DNA molecule that includes a sequence complementary to the protospacer sequence for the guide RNA. A gRNA is a component of the CRISPR/Cas system. The sequence specificity of a Cas DNA-binding protein is determined by gRNAs, which have nucleotide base-pairing complementarity to target DNA sequences. The native gRNA comprises a 20 nucleotide (nt) Specificity Determining Sequence (SDS), or spacer, which specifies the DNA sequence to be targeted, and is immediately followed by an 80 nt scaffold sequence, which associates the gRNA with the Cas protein. In some embodiments, an SDS of the present disclosure has a length of 15 to 100 nucleotides, or more. For example, an SDS may have a length of 15 to 90, 15 to 85, 15 to 80, 15 to 75, 15 to 70, 15 to 65, 15 to 60, 15 to 55, 15 to 50, 15 to 45, 15 to 40, 15 to 35, 15 to 30, or 15 to 20 nucleotides. In some embodiments, the SDS is 20 nucleotides long. For example, the SDS may be 15, 16, 17, 18, 19, 20, 21, 22, 23, 24, or 25 nucleotides long. At least a portion of the target DNA sequence is complementary to the SDS of the gRNA. For a Cas protein to successfully bind to the DNA target sequence, a region of the target sequence is complementary to the SDS of the gRNA sequence and is immediately followed by the correct protospacer adjacent motif (PAM) sequence. In some embodiments, an SDS is 100% complementary to its target sequence. In some embodiments, the SDS sequence is less than 100% complementary to its target sequence and is, thus, considered to be partially complementary to its target sequence. For example, a targeting sequence may be 99%, 98%, 97%, 96%, 95%, 94%, 93%, 92%, 91%, or 90% complementary to its target sequence. In some embodiments, the SDS of template DNA or target DNA may differ from a complementary region of a gRNA by 1, 2, 3, 4, or 5 nucleotides.

In some embodiments, the guide RNA is about 15-120 nucleotides long and comprises a sequence of at least 10 contiguous nucleotides that is complementary to a target sequence (e.g., a target sequence in Tpp1). In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 97, 98, 99, 100, 101, 102, 103, 104, 105, 106, 107, 108, 109, 110, 111, 112, 113, 114, 115, 116, 117, 118, 119, or 120 nucleotides long. In some embodiments, the guide RNA comprises a sequence of 10, 11, 12, 13, 14, 15, 16, 17, 18, 19, 20, or more contiguous nucleotides that is complementary to a target sequence. Sequence complementarity refers to distinct interactions between adenine and thymine (DNA) or uracil (RNA), and between guanine and cytosine.

### Linker

The term “linker,” as used herein, refers to a molecule linking two other molecules or moieties. The linker can be an amino acid sequence in the case of a linker joining two components of a fusion protein. For example, a napDNAbp (e.g., a Cas9 protein) can be fused to a deaminase (e.g., an adenosine deaminase) by an amino acid linker sequence. The linker can also be a nucleotide sequence in the case of joining two nucleotide sequences together (e.g., in a gRNA). In other embodiments, the linker is a non-peptidic linker. In some embodiments, the linker is an organic molecule, group, polymer, or chemical moiety. In some embodiments, the linker is 5-200 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

napDNAbp

As used herein, the term “nucleic acid programmable DNA binding protein” or “napDNAbp,” of which Cas proteins such as Cas9 and variants thereof are examples, refers to a protein that uses RNA:DNA hybridization to target and bind to specific sequences in a DNA molecule. Each napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the protospacer of a guide RNA). In other words, the guide nucleic-acid “programs” the napDNAbp (e.g., Cas9, or a variant thereof) to localize and bind to a complementary sequence.

Without being bound by theory, the binding mechanism of a napDNAbp-guide RNA complex, in general, includes the step of forming an R-loop whereby the napDNAbp induces the unwinding of a double-strand DNA target, thereby separating the strands in the region bound by the napDNAbp. The guide RNA protospacer then hybridizes to the “target strand.” This displaces a “non-target strand” that is complementary to the target strand, which forms the single strand region of the R-loop. In some embodiments, the napDNAbp includes one or more nuclease activities, which then cut the DNA, leaving various types of lesions. For example, the napDNAbp may comprise a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand.

### Nickase

As used herein, a “nickase” refers to a napDNAbp (e.g., a Cas9 protein) that is capable of cleaving only one of the two complementary strands of a double-stranded target DNA sequence, thereby generating a nick in that strand. In some embodiments, the nickase cleaves a non-target strand of a double stranded target DNA sequence. In some embodiments, the nickase comprises an amino acid sequence with one or more mutations in a catalytic domain of a canonical napDNAbp (e.g., a Cas9 protein), wherein the one or more mutations reduces or abolishes nuclease activity of the catalytic domain. In some embodiments, the nickase is a Cas9 that comprises one or more mutations in a RuvC-like domain relative to a wild type Cas9 sequence or to an equivalent amino acid position in other Cas9 variants or Cas9 equivalents. In some embodiments, the nickase is a Cas9 that comprises one or more mutations in an HNH-like domain relative to a wild type Cas9 sequence or to an equivalent amino acid position in other Cas9 variants or Cas9 equivalents. In some embodiments, the nickase is a Cas9 that comprises an aspartate-to-alanine substitution (D10A) in the RuvC1 catalytic domain of Cas9 relative to a canonical SpCas9 sequence or to an equivalent amino acid position in other Cas9 variants or Cas9 equivalents. In some embodiments, the nickase is a Cas9 that comprises an H840A, N854A, and/or N863A mutation relative to a canonical SpCas9 sequence, or to an equivalent amino acid position in other Cas9 variants or Cas9 equivalents. In some embodiments, the term “Cas9 nickase” refers to a Cas9 with one of the two nuclease domains inactivated. This enzyme is capable of cleaving only one strand of a target DNA. In some embodiments, the nickase is a Cas protein that is not a Cas9 nickase.

In some embodiments, the napDNAbp of a base editor is a Cas9 nickase (nCas9) that nicks only a single strand. In other embodiments, the napDNAbp can be selected from the group consisting of: Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (CasΦ), and Argonaute and optionally has a nickase activity such that only one strand is cut. In some embodiments, the napDNAbp is selected from Cas9, Cas12e, Cas12d, Cas12a, Cas12b1, Cas12b2, Cas13a, Cas12c, Cas12d, Cas12e, Cas12h, Cas12i, Cas12g, Cas12f (Cas14), Cas12f1, Cas12j (CasΦ), and Argonaute and optionally has a nickase activity such that one DNA strand is cut preferentially to the other DNA strand.

### Nuclear Localization Sequence (NLS)

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., international PCT application, PCT/EP2000/011690, filed Nov. 23, 2000, published as WO/2001/038547 on May 31, 2001, the contents of which are incorporated herein by reference for its disclosure of exemplary nuclear localization sequences. In some embodiments, a base editor comprises one or more NLS as described herein.

### Nucleic Acid Molecule

The term “nucleic acid,” as used herein, (also referred to as a “polynucleotide”) refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5 bromouridine, C5 fluorouridine, C5 iodouridine, C5 propynyl uridine, C5 propynyl cytidine, C5 methylcytidine, 7 deazaadenosine, 7 deazaguanosine, 8 oxoadenosine, 8 oxoguanosine, O(6) methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′ N phosphoramidite linkages).

### Protein, Peptide, and Polypeptide

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein, or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the contents of which are incorporated herein by reference.

### Protospacer

As used herein, the term “protospacer” refers to the sequence (~20 bp) in DNA adjacent to the PAM (protospacer adjacent motif) sequence. The protospacer shares the same sequence as the spacer sequence of the guide RNA. The guide RNA anneals to the complement of the protospacer sequence on the target DNA (specifically, one strand thereof, i.e., the “target strand” versus the “non-target strand” of the target DNA sequence). The skilled person will appreciate that the literature in the state of the art sometimes refers to the “protospacer” as the ~20-nt target-specific guide sequence on the guide RNA itself, rather than referring to it as a “spacer.” Thus, in some cases, the term “protospacer” as used herein may be used interchangeably with the term “spacer.” The context of the description surrounding the appearance of either “protospacer” or “spacer” will help inform the reader as to whether the term is in reference to the gRNA or the DNA target.

### Spacer Sequence

As used herein, the term “spacer sequence” in connection with a guide RNA refers to the portion of the guide RNA of about 20 nucleotides that contains a nucleotide sequence that shares the same sequence as the protospacer sequence in the target DNA sequence. The spacer sequence anneals to the complement of the protospacer sequence to form a ssRNA/ssDNA hybrid structure at the target site and a corresponding R loop ssDNA structure of the endogenous DNA strand.

### Subject

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex, and at any stage of development.

### Target Site

The term “target site” refers to a sequence within a nucleic acid molecule that is modified (e.g., edited) by a fusion protein disclosed herein (e.g., a base editor). The target site further refers to the sequence within a nucleic acid molecule (e.g., a nucleic acid molecule comprising Tpp1) to which a complex of, for example, a base editor and a gRNA binds.

### Treatment

The terms “treatment,” “treat,” and “treating,” refer to a clinical intervention aimed to reverse, alleviate, delay the onset of, or inhibit the progress of a disease or disorder (e.g., Batten disease), or one or more symptoms thereof, as described herein. As used herein, the terms “treatment,” “treat,” and “treating” refer to a clinical intervention aimed to reverse, alleviate, delay the onset of, or inhibit the progress of a disease or disorder (e.g., Batten disease), or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease (e.g., Batten disease). For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

### Tripeptidyl Peptidase 1 (Tpp1)

“Tripeptidyl peptidase 1 (Tpp1)” (also known in the art as lysosomal pepstatin-insensitive protease) is an enzyme encoded by the Tpp1 gene. Tpp1 functions in the lysosome to cleave N-terminal tripeptides from substrates. It also has peptidase activity. Mutations in Tpp1 may lead to Batten disease (e.g., CLN2).

The sequence of the human Tpp1 gene is provided below (GenBank Accession No. 001200), with the position at which a C·G-to-T·A transition mutation may result in an R208X mutation highlighted in bold:

(SEQ ID NO: 8)
AGCAGATCCGCGGAAGGGCAGAATGGGACTCCAAGCCTGGTGAGAAATTGAGA
GGGCTCGGGAGAAAGGGATCACGTTGGAGGGAGCACACATTGGGAGGGTGGGA
ACACAAGGACAACGTAGCTCCCACTGAAACCCACCTGCTGCCCCTACAGCCTCCT
AGGGCTCTTTGCCCTCATCCTCTCTGGCAAATGCAGTTACAGCCCGGAGCCCGAC
CAGCGGAGGACGTGAGTTGACTTAGCACAGACTGCCCCCTTCCCCATACCCTGTT
CTGCCTCCCACTGTCCTGGTCCTAGCTTCTCTCACCCCCGTGCCAGCTCCTAGTAC
GCACTCCATAGCTTCATCTCTATGTTCCTAGCCTCAGTCCCCTATCATTCACCTCA
TGTGATCTGTATCTGCTCCTAGGATCCTCCCCAAGGTCTCAGCTCCTAATCTGGA
ACCTTCCATGACCAATATTTTCCATCTCCACCCTAACCAAAGCCATGTCCCTGAC
CCCTGACCCTACAGGCTGCCCCCAGGCTGGGTGTCCCTGGGCCGTGCGGACCCTG
AGGAAGAGCTGAGTCTCACCTTTGCCCTGAGACAGCAGAATGTGGAAAGACTCT
CGGAGCTGGTGCAGGCTGTGTCGGATCCCAGCTCTCCTCAATACGGTGCCTTTTG
GGACTGAGGACAGGATGTGGGATGCGGTGGAGGGACACAGGGCTGGGTTGGGC
ATGGGATGGCGATCATGTCAGAGCCTGCCAAGACACTTGTGTTCCTCAGGCTAGA
AACCTAAAAGGGGATGTGGTTCAGTATACAGGCTTTATGATCAAATACGAACTC
AAATTCTGACTCTGCTACTTACTAGCTACCAGGCATCTAGTACAACTTACGTTCCT
TCCCCTACCCTCAAATCCATTCTAATTTCCTCTTTTGTTCACATACTAAGGCTGCC
AGTTCTAACTCCCAAAGAGCCTTTGGATTAATCTCCTTCTTGCCGTCTTTTGTTAC
GACCAACTCCGTTATTTATTCCCACTCCTGGACTACTGCCCAGCTCCCCAGCTGAT
CTGCAGTCTCCTCCCTCCACCCCACCCCTCACTGTACTCCCCCACTCTGCTTCAAA
ACAGTCTCTCCAACAGTCAAAATGGATTGGTTCCTTCTCCTGGTTAAAGCCCTTC
ACAGCAGGGGGAGTGTGTGCTTGTGAACTGCAGAGGCTGGGGACGGGGCAGTGT
GACACTAGTGTGCATGGCAGGAAACAGTGACTCACCATCGTGTTAAGCTTAAAA
TCAACAAGTATGCAAATTTGAGCTAAACTGAATAACTGGTACCGAAAGATTTGA
AAACATTTACCGGACATAAGCTTAAAATTAAAATGGAAAATTTATATGTATTAAC
CTCATTCATTTTCATGATCATGGCAATACATGCTGTGGTGCGACATTTGCAACTTA
CTGGCCTTTAGGGTAAATCCATATTATTGGCCATGGCATTTCAAGTCTCTCCAGC
CTTTTCTTCCTCTGCTTCCCAAGTACACCCTACACCTGCACACATAGCCCTCCTTT
ACCCTGTTCCAGGCTTTGGGAAGTCCTGATGTCTCATAGTTGAGGTCCAAAAGGG
GGAGTTTGGGAAAGCAATGAATGAGGGCAAGTGCCTCTTCTGAATCCCTGCAGG
AAAATACCTGACCCTAGAGAATGTGGCTGATCTGGTGAGGCCATCCCCACTGAC
CCTCCACACGGTGCAAAAATGGCTCTTGGCAGCCGGAGCCCAGAAGTGCCATTC
TGTGATCACACAGGACTTTCTGACTTGCTGGCTGAGCATCCGGTGAGAGGAAATG
ATTGCTCCATGGAGGGCACCAGTCATCCCATCAGTGAGATGGATGGGAGGGAGT
TGAGAGCTTGCTGGGGCTTGTGGGTGGGAGCTAATGCATGGGGAGACAGTGACT
GACTGCCCAGGGATGCTCAGAGGTAGCTTCTTCTGTTCCGTTTTGAGCTTTCTGAC
CTCTGTTCTCTGACCTCCAGACAAGCAGAGCTGCTGCTCCCTGGGGCTGAGTTTC
ATCACTATGTGGGAGGACCTACGGAAACCCATGTTGTAAGGTCCCCACATCCCTA
CCAGCTTCCACAGGCCTTGGCCCCCCATGTGGACTTTGGTAACACCTATGGGGTG
AATGGGGGTTGGGGCACACAGATCCAGGGGCTGAGGAAGTTTAGATGCCATTGG
GGACTGGGGGTGGGGTGAGTTGTAAGGTGGGCATTACAGTCTATAAGATCTCCTC
AAGCCTGACTTCTCCCTACAGTGGGGGGACTGCACCGTTTTCCCCCAACATCATC
CCTGAGGCAACGTCCTGAGCCGCAGGTGACAGGGACTGTAGGCCTGCATCTGGG
GGTAACCCCCTCTGTGATCCGTAAGCGATACAACTTGACCTCACAAGACGTGGG
CTCTGGCACCAGCAATAACAGCCAAGCCTGTGCCCAGGTGAGCCAAGCAAAGAG
CCCCAGGGTCCTCATAGCCTCCCCACAGTGTCCTCAATTCCTTACCACCCTGGGA
CTCACCCTCGGACCCACGATCTCTGCTCTGACTCCCTCCATAGTTCCTGGAGCAG
TATTTCCATGACTCAGACCTGGCTCAGTTCATGCGCCTCTTCGGTGGCAACTTTGC
ACATCAGGCATCAGTAGCCCGTGTGGTTGGACAACAGGGCCGGGGCCGGGCCGG
GATTGAGGCCAGTCTAGATGTGCAGTACCTGATGAGTGCTGGTGCCAACATCTCC
ACCTGGGTCTACAGTAGCCCTGGTACTACCAAGAGGACTGGACAGTGGGGAAGG
GGGTGGGAGATGGGTGTTGATCCCTGCTCCCTCAAGGGAATGCTATAAGCTGGA
GAGAGATCCTGACAACCCCCAGTGACTATCTTTGTGCCCATCCCTCAAAAAAAAA
AAAAAAAAAAATCCAGGCCGGCATGAGGGACAGGAGCCCTTCCTGCAGTGGCTC
ATGCTGCTCAGTAATGAGTCAGCCCTGCCACATGTGCATACTGTGAGCTATGGAG
ATGATGAGGACTCCCTCAGCAGCGCCTACATCCAGCGGGTCAACACTGAGCTCA
TGAAGGCTGCCGCTCGGGGTCTCACCCTGCTCTTCGCCTCAGGTGACCTCCTACC
CTAAACTTAGACAATGCTTACACCTCTGCAGCCTGGGAGCTTTGACTCCACAGTG
ATCCCTGAGCCTGGTCTCTGACTCATAATCTGAACTCAGACCTTCCAGTAGGGAC
CACTGACCTGACCTCTACACTCTGACCTCCTACAGTAACAAATTTCCCCTCTGAC
ATCCGAACCCACATACTAAGCCCTAACCAATTAATATGAATGCTACACTTGGTCT
CTCTCAGGTGACAGTGGGGCCGGGTGTTGGTCTGTCTCTGGAAGACACCAGTTCC
GCCCTACCTTCCCTGCCTCCAGGTAAGTACTCTAGCCTACCACTCAGGTATAACC
ACCACCTTTCACTTGTGATCTCATGATGTAGAACCTTTGTCTTGACCCCACCATGT
GCTCCTGTGGTTCAGCCTTAAGCTTTGCCTGCCCTGGTTGCTGTACTCCTGTCTCT
TCTTCCTGCAGGTCCCAGGCCCCAAATCTCTTGTGTGGGATACAGCTCCCATTGTT
CCTTTTCGTCAGTTCCCAGGCATTTTAGTGGAAGATTTGGTGGGTGTTCTGTAGAG
AAAAGTGTGCACAGTCACCTCGGGCCATGCCTTGAAGGCTCAAAATCTCTTAGTC
AATCCCATATACATGCTTCCCCACAGAGTCTAGTTCCTCCAGCAAGACCTGGGCT
ATACTCACCCCTCCCCACATATCTTGGAGGTCCCCTTGGGTCCCCTACTATCCAA
ATGCTGTCTTCTCCCCTCAGCCCCTATGTCACCACAGTGGGAGGCACATCCTTCC
AGGAACCTTTCCTCATCACAAATGAAATTGTTGACTATATCAGTGGTGGTGGCTT
CAGCAATGTGTTCCCACGGCCTTCATACCAGGTACGTGTGTTTGTGTGGATGGAT
GCAGGGTAAGAGTGAGGATGGGGGATCCTCAGTTCAGCTGACTGCTGGGCAGGC
CACATGCCAATACTCACTCAAAAATGCCTTTCAGGAGGAAGCTGTAACGAAGTT
CCTGAGCTCTAGCCCCCACCTGCCACCATCCAGTTACTTCAATGCCAGTGGCCGT
GCCTACCCAGATGTGGCTGCACTTTCTGATGGCTACTGGGTGGTCAGCAACAGAG
TGCCCATTCCATGGGTGTCCGGAACCTCGGTGAGAATCAGCCCATCTCCAAACTC
TCACTCAGGAACTACCCTTACCCCCTAACACCTTGAACACCTTGCACCTAGAACC
CCTGACTCCTTAGAGATGTCTGATACTTTAAAGCATCACTCCCAAAAAGTCCAAT
CACTCAGAACCCCTGACCTCTACTTGCACCTTCACTCTTGTAGGCCTCTACTCCAG
TGTTTGGGGGGATCCTATCCTTGATCAATGAGCACAGGATCCTTAGTGGCCGCCC
CCCTCTTGGCTTTCTCAACCCAAGGCTCTACCAGCAGCATGGGGCAGGACTCTTT
GATGTAAGTATGGAAGGGAAGGGTGTGGACGTTTTCAAACAACTATGGGGAGTG
CTAAGGGGGACTTGGGGGCAGTTAGGGTGGTGTGGAATAGCCTTTGAAATGTGA
GTACAGGGTGAGGAGATATACTCTTTAAGTACTGGTACTAGTAGGCCCAGATCTG
ATGCCAGCCTCCTCCCTAGGTAACCCGTGGCTGCCATGAGTCCTGTCTGGATGAA
GAGGTAGAGGGCCAGGGTTTCTGCTCTGGTCCTGGCTGGGATCCTGTAACAGGCT
GGGGAACACCCAACTTCCCAGCTTTGCTGAAGACTCTACTCAACCCCTGACCCTT
TCCTATCAGGAGAGATGGCTTGTCCCCTGCCCTGAAGCTGGCAGTTCAGTCCCTT
ATTCTGCCCTGTTGGAAGCCCTGCTGAACCCTCAACTATTGACTGCTGCAGACAG
CTTATCTCCCTAACCCTGAAATGCTGTGAGCTTGACTTGACTCCCAACCCTACCAT
GCTCCATCATACTCAGGTCTCCCTACTCCTGCCTTAGATTCCTCAATAAGATGCTG
TAACTAGCATTTTTTGAATGCCTCTCCCTCCGCATCTCATCTTTCTCTTTTCAATCA
GGCTTTTCCAAAGGGTTGTATACAGACTCTGTGCACTATTTCACTTGATATTCATT
CCCCAATTCACTGCAAGGAGACCTCTACTGTCACCGTTTACTCTTTCCTACCCTGA
CATCCAGAAACAATGGCCTCCAGTGCATACTTCTCAATCTTTGCTTTATGGCCTTT
CCATCATAGTTGCCCACTCCCTCTCCTTACTTAGCTTCCAGGTCTTAACTTCTCTG
ACTACTCTTGTCTTCCTCTCTCATCAATTTCTGCTTCTTCATGGAATGCTGACCTTC
ATTGCTCCATTTGTAGATTTTTGCTCTTCTCAGTTTACTCATTGTCCCCTGGAACA
AATCACTGACATCTACAACCATTACCATCTCACTAAATAAGACTTTCTATCCAAT
AATGATTGATACCTCAAATGTAAGATGCGTGATACTCAACATTTCATCGTCCACC
TTCCCAACCCCAAACAATTCCATCTCGTTTCTTCTTGGTAAATGATGCTATGCTTT
TTCCAACCAAGCCAGAAACCTGTGTCATCTTTTCACCCCACCTTCAATCAACAAG
TCCTCAATCAACAAGTCCTACTGACTGCACATCTTAAATATATCTTTATCAGTCCA
CAAGTCCTTCCAATTATATTTCCCAAGTATATCTAGAACTTATCCACTTATATCCC
CACTGCTACTACCTTAGTTTAGGGCTATATTCTCTTGAAAAAAAGTGTCCTTACTT
CCTGCCAATCCCCAAGTCATCTTCCAGAGTAAAATGCAAATCCCATCAGGCCACT
TGGATGAAAACCCTTCAAGGATTACTGGATAGAATTCAGGCTTTCCCCTCCAGCC
CCCAATCATAGCTCACAAACCTTCCTTGCTATTTGTTCTTAAGTAAAAAATCATTT
TTCCTCCTCCCTCCCCAAACCCCAAGGAACTCTCACTCTTGCTCAAGCTGTTCCGT
CCCCTTACCACCCCTGATACAACTGCCAGGTTAATTTCCAGAATTCTTGCAAGAC
TCAGTTCAGAAGTCACCTTCTTTCGTGAATGTTTTGATTCCCTGAGGCTACTTTAT
TTTGGTATGGCTGAAAAATCCTAGATTTTCTAAACAAAACCTGTTTGAATCTTGG
TTCTGATATGGACTAGGAGAGAGACTGGGTCAAGTAAGCTTATCTCCCTGAGGCT
GTTTCCTCGTCTGTTAAGTGTGAATATCAATACCTGCCTTTCATAATCACCAGGG
AATAAAGTGGAATAATGTTGATAACAGTGCTTGGCACCTGGAAGTAGGTGGCAG
ATGTTAACGCCCTTCCTCCCTTGCACTGCGCCCCCTGTGCCTACCTCTAGCATTGT
AACGACCACGTAGTATTGAAATGGCCAGTTTACTTGTCTGCCTTCCTTTCCAAGA
CCGTTGGTGCCTAGAGGACTAGAATCGTGTCCTATTTAACTTTGTGTTCCCAGGT
CCTAGCTCAGGAGTTGGCAAATAAGAATTAAATGTCTGCTACACCGAAAA

The sequence of the human Tpp1 enzyme is provided below, with R208 highlighted in bold:

(SEQ ID NO: 9)
MGLQACLLGLFALILSGKCSYSPEPDQRRTLPPGWVSLGRADPEEELSLT
FALRQQNVERLSELVQAVSDPSSPQYGKYLTLENVADLVRPSPLTLHTVQ
KWLLAAGAQKCHSVITQDFLTCWLSIRQAELLLPGAEFHHYVGGPTETHV
VRSPHPYQLPQALAPHVDFVGGLHRFPPTSSLRQRPEPQVTGTVGLHLGV
TPSVIRKRYNLTSQDVGSGTSNNSQACAQFLEQYFHDSDLAQFMRLFGGN
FAHQASVARVVGQQGRGRAGIEASLDVQYLMSAGANISTWVYSSPGRHEG
QEPFLQWLMLLSNESALPHVHTVSYGDDEDSLSSAYIQRVNTELMKAAAR
GLTLLFASGDSGAGCWSVSGRHQFRPTFPASSPYVTTVGGTSFQEPFLIT
NEIVDYISGGGFSNVFPRPSYQEEAVTKFLSSSPHLPPSSYFNASGRAYP
DVAALSDGYWVVSNRVPIPWVSGTSASTPVFGGILSLINEHRILSGRPPL
GFLNPRLYQQHGAGLFDVTRGCHESCLDEEVEGQGFCSGPGWDPVTGWGT
PNFPALLKTLLNP

In some embodiments, the present disclosure provides gRNAs targeting the mouse Tpp1 gene. The corresponding position of the human R208X mutation in the mouse Tpp1 enzyme is R207X. The sequence of the mouse Tpp1 gene is provided below (GenBank Accession No. 12751), with the position at which a C·G-to-T·A transition mutation may result in an R207X mutation highlighted in bold:

(SEQ ID NO: 10)
GCAACGTCACTAGTTACTAGGCAGGGAGATGGGGGAGGTGCCAGACCATGCTCA
TGTGACTTATCACATGACTACAGATCAGCCTGAAAGCCAAAATGGGACTCCAAG
CCCGGTGAGAAACTGAGGTGGGGAGGGTAGCTAAGGGGGATTACATTAGAGGG
ATTACATGTTGTAGAAGGAGTACAATGAGGGTTCGGCTCCTGATGAAACCCACCT
GCTGTCCCTGCAGCCTCCTAGGGCTCCTTGCTCTCGTCATCGCCGGCAAATGCAC
TTACAACCCTGAGCCGGACCAGCGGTGGATGTGAGTTGATTTAGTACTACAGGTG
CCTGTCTCTGCAGACGCTTGGTCCTAGCTTTCCTCATCCTCAACTGCTCCTAGTAC
CCACTCTGTAGCCTTATTCGACACCCCCTCGGCTTAATCTCTGGTCATTTATCTCC
TATGATCCTTATCTGCATCTAGGATTCGACCCAAAGTCTCAGTTCTTAATCCCAA
ACTTTCCATGATCTGGATTTCTCGTTATGGCCTAATCAAATCATGTCCCTGACCCT
GTAGGCTGCCTCCAGGCTGGGTGTCCCTGGGCCGCGTGGATCCCGAGGAAGAGC
TGAGTCTCACTTTTGCGCTGAAACAGCGGAACCTGGAAAGACTCTCGGAGCTGGT
GCAGGCTGTGTCGGATCCTAGCTCTCCTCAATATGGTGCCTATCAGTTGGGGCAG
GAAGTGGGAGGCAGGACCAGGCAGGCACTGGCTGGTGCAGATCATGTAGGGCC
ATGTTTCTGGTATGACAGACAAATGACAATAACTGCCATATTCATACACACACAC
ACACACACACACACACACACACACACACACCAAAATTTCCACCTGAGTTCTTCA
GTCTTAAAACTAAGAACAAATCTAAGCCTAGTAGCATGAGGTAATAATCCTAGC
TACTTGGACTAAAACAGGACGATTTTGAATCCAAGGCATGCCTGGGCAGTGTAG
CAATATTGTCAACATTAAAACCAATTTTTTTTAGAGGCCTAGCTAGCTCATTGAT
AGGGTGGGTGCTTACGGAGCATATGAAAAACCCTAGGTTCAAGTGCCAGGAAGA
TGGTGGGTGTGGGGTAGCTAGCTGTGTTTAAGAACACATGATTCATAATCAGATC
TAGATTGAAATGTTCACTTACCCTACCAGCTCTCAAATGGAGGACTAGCACTTAG
TGTGTGTGCATGATCAAGTCATGGACCTTACCCCACTCTCATAGCCACTGCCACA
CCCTCCTCTGTTGCTAGTTCAAGTCCTAAATTGTCTTTGGATTAATCCACTCTGGC
CATTCCTCTTGCTACCAACTCAGTTGTTTATTCTCTTTCTGGATTACTGCCCAGTA
CCCGGACTGGTTTGAAGCTTCCTCTCCTTCACCCCACTCCCCAGAACAGTCCCTTC
TCTATTCTAGAGTATTCTCTATGGTGCTCAGGCTGGAGTGAATCCTTCCCCTGCTT
AAAGTCCTGTATGGTGAGGCAAGGGTGTGTGTGTGTGTGTGTGTGTGTAGAATGG
AGGCGAGAGGAGGGGGGGGCATCCTTAGAGCACCCAAGAAGCTCCTCATATGCA
GGAAAGAATGACTCACCGTTATGTTGTTTAAAATCAACAAGGATGAAGAGCTGA
GCTAAATTAGAAGGTTCTGTATAACTGGCTCCAAAAGTTTGGGGAAATCTTTTTG
AAGATAAGCTTAAAATGGGATATTGAAAGTTTACTAACGTCCTTGAGTTTTTGTG
ACCATGGTAACACATGCTGTGCATTTTTCCTGGCCATGACGTTTCATGTCTCTCGA
GCCTTTTCTTCCCTTTGACTCTCACTATGCCTAGCCCTACACAGTCCTCCTCGGGA
AGTTTTGATGTCCTCTGATTGAGGTCCAGACAGACAACCCAGGAAAGCCTCAAAT
GAGGACGAGAGCCTCTTCTGAATCCCTGCAGGAAAGTACCTAACCCTGGAGGAT
GTAGCTGAGCTGGTTCAACCATCACCCCTGACCCTCCTCACTGTCCAAAAGTGGC
TCTCAGCAGCTGGAGCCCGGAACTGCGATTCAGTGACCACCCAGGACTTTCTGAC
TTGCTGGCTGAGTGTCCGGTGAGAAGTAATGATTTCCCCATAGAATCCATTGTCC
CTACAAGGAGACAAAATACCCCAATTGGGGGAGTTTAAAGCGTGCTGGGAGCCT
GTGGGTATAGGTAATGCATACGGAAATGATGGCAGGCTGCTAAGTGGGGCTCAG
GCCAACTCTTCTACTCCTTCCCATGGTTTCTGTTTTCTTACTTCCAGACAGGCTGA
GCTGCTGCTCCCAGGAGCTGAGTTTCATCGCTATGTAGGGGGACCTACAAAGACC
CATGTTATAAGGTCCCCACATCCCTACCAGCTTCCCCAGGCCTTGGCCCCTCATG
TGGATTTTGGTAAACCCAATGGAGTTGGTGGAAGTTGTGGGAGGGAGGTCTACA
GGCTGAAGAATTTTAGATGCCAAAAGAGGCATAGAATCTTTCCAGTAGAGAAGT
GGGTGGTAGTGTCTGTAAGACCTCCTTAAGCCTGACCCCTTTCCACAGTGGGGGG
GCTGCACCGTTTCCCCCCTTCATCTCCAAGACAACGTCCAGAACCACAACAGGTA
GGAACTGTTAGCCTGCACTTGGGAGTGACTCCGTCTGTGCTCCGTCAGCGATACA
ACCTGACAGCCAAAGATGTGGGCTCAGGCACCACCAACAATAGCCAGGCCTGTG
CCCAGGTGAGCCATGTAAAGCCCTGCGGTCATCACAACCTCCTCAAGATATCTTT
AGTGCCTCACTACCCTGGGCCTCACTCTCTGATCCACAATTCCTGATTTGGATGTT
TCCACAGTTCCTGGAACAGTACTTCCATAACTCGGATCTGACTGAGTTCATGCGC
CTATTCGGTGGCAGTTTTACACACCAGGCCTCAGTAGCAAAAGTTGTTGGAAAGC
AAGGGCGAGGCCGAGCTGGGATCGAGGCCAGTCTAGATGTGGAATACCTGATGA
GTGCTGGTGCCAATATCTCCACTTGGGTCTACAGTAGCCCTGGTATTGCTAAGAG
AATTAGTTGGGGGATGGGAAAATGGGTTGGAGTAGACTTTTGGTCTCTGCTTCAT
TTCATCAAGGGGATGCCATGGGCTGAAGGGAGATTCTAGCAACCATCCAATGGC
CATTCATATCCCTTCTTTTAAAACAATTCAGGCCGCCATGAGGCACAGGAGCCCT
TCTTACAATGGCTCCTGCTTCTTAGCAATGAGTCATCTTTGCCACATGTACATACT
GTGAGTTACGGAGACGATGAAGACTCCCTCAGCAGCATCTACATCCAGAGAGTC
AACACTGAGTTCATGAAGGCTGCTGCTCGGGGTCTCACCCTCCTTTTTGCCTCAG
GTAACCTTCTACCATAAATTTAAGACTTCCCACCTACCCAAGCGGCAGACTTTAT
CCCAACCGACCCTTCAGCCTGGTTTCTGACTCATAATAGGAACTCAGAGCCTTAA
CAGGGTCTGCTGATTTGACCTGAGTACTCTGAGGTGGCATATACAGTTCTTCTCT
GGTATATAGAAGCCTACACCCCAAGTTCTTCACAACTAATCTGAATACTTACTAT
GTCTTTCCCAGGTGACACTGGAGCTGGGTGTTGGTCTGTCTCCGGAAGACACAAG
TTCCGCCCTAGCTTCCCTGCTTCCAGGTAAGTACCCCACTCTTTCACTTGTGACAG
AGGCCACCAGGAGCTGCTGTGGCTCAGCCTTCAGCATTACCTGTTGTGTTTGCTG
TGCTCCCTTTTCTTCCTGCGTATCCCAGGCTGCGAGAGCAGATTATGGCGTTTCTT
TTCCTTAGTTGCTAGGGTTTGTTGCTTGTTTTTCATGTAGAAAAGTATATACAATT
AACTCCAGCCATGTCTTGAGAGCTCCCAATCTATCAATAAACTCTGTATACAGGC
TTCTATAGTCTTACTCCCTTTTCCAGTAAGACCCAGACCATTCCCACCCACCTCCA
CACATCTTGGAGGTCACCCATTGTCTTAGTCGGGGTTTTTATTGTATTGCTGCGAT
GAAACACCGTGACAAAAAAACAAAACAAAACAAAACAAAACAAAAACAGTTGA
GGAGGAAAGGGTTTATTTGGCTTACACTTCCAGATCACATCCGTCACTGAAGGAA
ATCAGGACAGAAACTCAAGCAGGGCTGGAACCTGGAAGCAGAAGCTGATGAAG
AGGCCAGGGAATGGTGCTGCTTACTGGCTTGCTTCCCATGGCTTGTTCAGCCTGC
CATCTTATAGAACCCACGACCATCAGCCCAGGGATGCCACCCTCCACAATGGGCT
GGGCCCTCCCTCATTGATCACTAATTGAGAAAATGTCCTACAGCTGGATCTCATG
GAGGCATTTCCTTAACTGAGCTTCCTTTGTCTCTGATGACTCTTGTATCAAGTTGA
CAACACAAAACTAACCAGCAAGTACATTCACTATCTGAATACTGTCTTCTCCTCA
GCCCCTATGTTACTACAGTTGGAGGAACCTCCTTCAAGAATCCTTTCCTCATCAC
AGATGAAGTAGTTGACTATATCAGTGGTGGAGGCTTCAGCAATGTTTTCCCACGG
CCTCCCTACCAGGTTTGTGGATATTCCTGTGGATATCTGGAGGTTGAAGGTGATG
GGTGGGGCTCAGTCCTGCAGCTTGCTGAGCAGGCTGCTGGCCAATACTCATACTC
AGAAATGTCCTTCAGGAGGAAGCAGTGGCCCAGTTCTTGAAATCCAGCTCTCATC
TACCACCATCCAGTTACTTCAATGCTAGTGGCCGTGCCTACCCAGATGTTGCCGC
ACTATCTGATGGCTACTGGGTGGTCAGCAACATGGTCCCCATTCCATGGGTATCT
GGAACCTCGGTAAGAATCAGCTCTGCTCTAAACGCTCTACTCAGGAACTACCCTC
GCTCCTCCACCTACACAATCTAAACGCTCTACTCAGGAACTACCCTCGCTCCTCC
ACCTACACAATCTAAACGCTCTACTCAGGAACTACCCTCGCTCCTCCACCTACAC
AATCTAAACGCTCTACTCAGGAACTACCCTCGCTCCTCCACCTACACAATCTTGA
ACCCAGAACCCCCGACTCCTTGGAGACTCCTGATCTTTGCAAGCATCATCCCTTA
GAAGTCCAATCCCTCTAAAACCCTAACCTATTCTTGCATCTTCATCTTGCAGGCCT
CTACTCCAGTGTTTGGGGGAATTTTATCCTTGATAAATGAGCACAGAATCCTCAA
TGGCCGCCCTCCTCTTGGCTTTCTCAACCCCAGGCTCTATCAGCAGCATGGGACA
GGACTCTTTGATGTGAGTATTGGAGGAAAGAGTGTGGATGTTGTCATAGGATATG
AGAAGGGCTCTGGTGAACTTCGGCATTTTCACTATCTATGATTGCCTCTGTGATAT
GACTATAAATAAGGTGCAGTCTAGGAGCTGGTACCAGCAGGCCCAGACCTGATG
CCATCATCTCCTCCCAGGTAACCCACGGCTGCCATGAGTCCTGTCTGAATGAAGA
AGTGGAGGGTCAGGGTTTCTGCTCTGGTCCTGGCTGGGATCCTGTGACAGGTTGG
GGAACACCCAACTTCCCAGCCCTACTGAAGACCCTGCTCAACCCTTGACCCTTTC
GTGCCATGACGAGAAAGCAGAACTGTTCCCTGTACTAAAAGGGAAGGCTCAGTT
TCTTGTTATTCCTCGATAGAAGCCCTGCTGAACTCCTGTTGCCTGCTGCAGATAGC
TTCTCCCTAACCCTCAGATGCTGTGAACAGGACTCAACTCTCAATCCTACTGTGT
GCCATCAAACTCAGGTCTCCAAACTTCTACTTCAAGATCCTCAACAAGATGCTAT
AACCAGCATATTTTGTCTCACCCCAACCCCATCTCTCCTTCCTCTTTCCAGCTTGA
GATGTGAAAGCAGGGCAAGAAGGTTCAGTCTTCCATTACTGACACTAGCAGGTC
CACCCAACGCTTACCACCTCTGCACTGACCGTACACTCTATTTCTCTTCGGGTTTG
CTTTTCCGTTCACTGAAGTGAGACCTTTGACTAATCGTTTTGTCTTTCTTCTCTCGG
CACTGAAGTACAATGGTCTCCCCAATGTTTTATCCAGTTATACCCTTTTCAGTGTT
TGTTTTATGGGTTTTCTTATTTAAGAACAGGTTGTCAAAAAACCATTAAAAAAAA
AAAAGAAAAAGAACACATTGGCTGCAATCTATTTAACTATATAACTATTCTAAGG
AAAGTTAAAAATTGAAAACTTAAAATGTTTGAAATGTTCTCATTGGCAAAATTCC
TCAACAAAATAAATAGGTCATTACAAATTTTGCTTTAAATTTTTGCTTGAGTGATT
TTTTTTTTTGTAAAGTGTTTTAAAATTTACATTTTATTTTCCCCTTCTCAGCTACAC
CAAAGCAACATTCAGGTTTTAATTTCAACTGTCAGACATTACAAACATCTAGCTT
CTGTGAACCTGGGTGTTTGTTCTTTCCATCAGTTTCCCATTTATCCTTTTTCCTCAG
TGACCCCCCTCTGACCACACTAATCAGCCCCCTTTTCTGCTGACTCCAGGGATGC
TGTGTTCATTGTTCCATTCTTAGTTTCTTGCTCTTATCATTTTATTATCATCCCTTG
ACAGATCACTGACATCTATACCCACAGTGAGTGATACCTCAAATGTGAGATACCT
CGTTACTTCATCTCCCTCCAGCCCAGACCTAAACTATTTCATCTCTTAATCTATGA
TATAATGCCTCTTTCAAACAAGCCAGAAACCTATAACTCTTAACTCTCATCTTTTT
CACCTCCTTACTAACTTCAAAAAGTTTTCTTGACTGCCTCTCAAGTATATCTTTAT
CGGCTAGTGTGGTGATGCACACCTTTAATCCCAGCACTCAGGAGACAGAGGCCA
GGCAAGTCTCTGAGTTTGAGGCCATCCTGGTCTATATAAAGAGTTCCAGACAAGC
CAGGCCTACATAGTCAGACCATGTCTCAAAAATACACATATGTGCACACACACA
CATGCACAAAATACTGCATTATCTTTGAGCCCATGTTCTTTTTTCTTCTAAACTGC
AGGAAGCACTTGGGGAGAGGAAAGGCTTCCGAAGCCCCTCATCTTCCAAACCAA
GCAGTCTATGTATTTGTGCAAACCCTTCAAAGATTACTGGGTAAAAGTCAGAGAC
ATTGAAACTTGCCTTCAAAATCGGGAAATAAACATTGCTAATGTCTCACACTTGG
AATTAAGCAATGAATGTTAGTTTCCCTTTCTTCTTTGCACTGCACCTACATGTAGC
TGGGAAACAATCATACAGTATTGAAATTGTCAGTTTGTTTGCCTTCCTTTTCCAGA
CAGTCGGTGCTAGTGAACTAGAATCTGATTCTTAACTTTGTATTCCTAGGTCCCA
GCCCAAATAGAAATTAAATAAATGACTGTTTAAAAAAAAA

The sequence of the mouse Tpp1 enzyme is provided below, with R207 highlighted in bold:

(SEQ ID NO: 11)
MGLQARLLGLLALVIAGKCTYNPEPDQRWMLPPGWVSLGRVDPEEELSLT
FALKQRNLERLSELVQAVSDPSSPQYGKYLTLEDVAELVQPSPLTLLTVQ
KWLSAAGARNCDSVTTQDFLTCWLSVRQAELLLPGAEFHRYVGGPTKTHV
IRSPHPYQLPQALAPHVDFVGGLHRFPPSSPRQRPEPQQVGTVSLHLGVT
PSVLRQRYNLTAKDVGSGTTNNSQACAQFLEQYFHNSDLTEFMRLFGGSF
THQASVAKVVGKQGRGRAGIEASLDVEYLMSAGANISTWVYSSPGRHEAQ
EPFLQWLLLLSNESSLPHVHTVSYGDDEDSLSSIYIQRVNTEFMKAAARG
LTLLFASGDTGAGCWSVSGRHKFRPSFPASSPYVTTVGGTSFKNPFLITD
EVVDYISGGGFSNVFPRPPYQEEAVAQFLKSSSHLPPSSYFNASGRAYPD
VAALSDGYWVVSNMVPIPWVSGTSASTPVFGGILSLINEHRILNGRPPLG
FLNPRLYQQHGTGLFDVTHGCHESCLNEEVEGQGFCSGPGWDPVTGWGTP
NFPALLKTLLNP

### Variant

As used herein, the term “variant” should be taken to mean the exhibition of qualities that have a pattern that deviates from what occurs in nature, e.g., a variant Cas9 is a Cas9 comprising one or more changes in amino acid residues (i.e., “substitutions”) as compared to a wild type Cas9 amino acid sequence. The term “variant” encompasses homologous proteins having at least 80%, at least 85%, at least 90%, at least 95%, at least 96%, at least 97%, at least 98%, or at least 99% identity with a reference sequence and having the same or substantially the same functional activity or activities as the reference sequence. The term also encompasses mutants, truncations, or domains of a reference sequence that display the same or substantially the same functional activity or activities as the reference sequence.

### Vector

The term “vector,” as used herein, refers to a nucleic acid that can be modified to encode a gene of interest and that is able to enter a host cell, mutate, and replicate within the host cell, and then transfer a replicated form of the vector into another host cell. Exemplary suitable vectors include viral vectors, such as retroviral vectors or bacteriophages and filamentous phage, and conjugative plasmids. Additional suitable vectors will be apparent to those of skill in the art based on the instant disclosure.

### DETAILED DESCRIPTION

The present disclosure describes the use of adenosine base editors and gRNAs for editing the Tpp1 gene to correct an R208X mutation in the Tpp1 protein and treat Batten disease (i.e., CLN2). Methods of editing Tpp1 using a base editor and a gRNA are provided herein. Such methods may be useful for treating Batten disease. The present disclosure also provides gRNAs and base editor-gRNA complexes for editing Tpp1 and treating Batten disease. Polynucleotides, vectors, AAV particles, cells, and kits for editing Tpp1 and treating Batten disease are also provided herein.

Guide RNAs (gRNAs)

The present disclosure provides gRNAs for targeting a genome editing agent (e.g., a base editor) to a Tpp1 gene (e.g., a human or mouse Tpp1 gene). The gRNAs provided herein may be useful for treating Batten disease.

In some embodiments, the gRNAs target a base editor to a site in the human Tpp1 gene of SEQ ID NO: 8. In some embodiments, the gRNAs target a base editor to a site in the human Tpp1 gene such that the base editor corrects a C·G-to-T·A transition mutation in the Tpp1 gene, leading to correction of an R208X mutation in the human Tpp l enzyme (where X is a premature stop codon).

In some embodiments, the gRNAs provided herein target a strand complementary to a protospacer in a Tpp1 gene comprising a nucleotide sequence at least 80%, at least 85%, at least 90%, at least 95%, at least 96%, at least 97%, at least 98%, or at least 99% identical to the sequence TATCACTTACGGATCACAGA (SEQ ID NO: 2), or a sequence comprising one, two, three, four, or five mutations relative to TATCACTTACGGATCACAGA (SEQ ID NO: 2), or a fragment thereof. The provided gRNAs may also target a sequence in a Tpp1 gene that is shifted upstream or downstream relative to SEQ ID NO: 2 in the human Tpp1 gene of SEQ ID NO: 8 (e.g., shifted upstream or downstream by 1, 2, 3, 4, 5, 6, 7, 8, 9, or 10 nucleotides). In certain embodiments, the provided gRNAs target a strand complementary to a protospacer in the Tpp1 gene comprising the nucleotide sequence TATCACTTACGGATCACAGA (SEQ ID NO: 2). In some embodiments, the gRNAs provided herein comprise a spacer targeting the gRNA to a human Tpp1 gene. In some embodiments, the gRNAs comprise a spacer at least 80%, at least 85%, at least 90%, at least 95%, at least 96%, at least 97%, at least 98%, or at least 99% identical to the sequence GTATCACTTACGGATCACAGA (SEQ ID NO: 4), or a fragment thereof. In certain embodiments, the gRNAs comprise a spacer of the sequence GTATCACTTACGGATCACAGA (SEQ ID NO: 4).

In some embodiments, the gRNAs target a base editor to a site in the mouse Tpp1 gene of SEQ ID NO: 10. In some embodiments, the gRNAs target a base editor to a site in the mouse Tpp1 gene such that the base editor corrects a C·G-to-T·A transition mutation in the mouse Tpp1 gene, leading to correction of an R207X mutation in the mouse Tpp 1 enzyme (where X is a premature stop codon).

In some embodiments, the gRNAs provided herein target a strand complementary to a protospacer in a Tpp1 gene comprising a nucleotide sequence at least 80%, at least 85%, at least 90%, at least 95%, at least 96%, at least 97%, at least 98%, or at least 99% identical to the sequence TATCACTGACGGAGCACAGA (SEQ ID NO: 1), or a fragment thereof, or a sequence comprising one, two, three, four, or five mutations relative to TATCACTGACGGAGCACAGA (SEQ ID NO: 1). The provided gRNAs may also target a sequence in a Tpp1 gene that is shifted upstream or downstream relative to SEQ ID NO: 1 in the mouse Tpp1 gene of SEQ ID NO: 10 (e.g., shifted upstream or downstream by 1, 2, 3, 4, 5, 6, 7, 8, 9, or 10 nucleotides). In certain embodiments, the provided gRNAs target a strand complementary to a protospacer in the Tpp1 gene comprising the nucleotide sequence TATCACTGACGGAGCACAGA (SEQ ID NO: 1). In some embodiments, the gRNAs provided herein comprise a spacer targeting the gRNA to a mouse Tpp1 gene. In some embodiments, the gRNAs comprise a spacer at least 80%, at least 85%, at least 90%, at least 95%, at least 96%, at least 97%, at least 98%, or at least 99% identical to the sequence GTATCACTGACGGAGCACAGA (SEQ ID NO: 3), or a fragment thereof. In certain embodiments, the gRNAs comprise a spacer of the sequence GTATCACTGACGGAGCACAGA (SEQ ID NO: 3).

The gRNAs provided herein also comprise a gRNA backbone sequence that facilitates binding of the gRNA to a napDNAbp, for example, a Cas9 protein (e.g., a Cas9 protein as part of a base editor). In some embodiments, the provided gRNAs comprise a gRNA backbone sequence for binding to SpCas9. In some embodiments, the gRNAs comprise a backbone scaffold at least 80%, at least 85%, at least 90%, at least 95%, at least 96%, at least 97%, at least 98%, or at least 99% identical to the sequence GTTTTAGAGCTAGAAATAGCAAGTTAAAATAAGGCTAGTCCGTTATCAACTTGAA AAAGTGGCACCGAGTCGGTGC (SEQ ID NO: 5), or a fragment thereof. In certain embodiments, the gRNAs comprise a backbone scaffold of the sequence GTTTTAGAGCTAGAAATAGCAAGTTAAAATAAGGCTAGTCCGTTATCAACTTGAA AAAGTGGCACCGAGTCGGTGC (SEQ ID NO: 5), or a fragment thereof.

In some embodiments, the present disclosure provides gRNAs comprising sequences at least 80%, at least 85%, at least 90%, at least 95%, at least 96%, at least 97%, at least 98%, or at least 99% identical to the sequence GTATCACTTACGGATCACAGAGTTTTAGAGCTAGAAATAGCAAGTTAAAATAAG GCTAGTCCGTTATCAACTTGAAAAAGTGGCACCGAGTCGGTGC (SEQ ID NO: 6) or GTATCACTGACGGAGCACAGAGTTTTAGAGCTAGAAATAGCAAGTTAAAATAAG GCTAGTCCGTTATCAACTTGAAAAAGTGGCACCGAGTCGGTGC (SEQ ID NO: 7). In certain embodiments, the gRNA comprises the sequence GTATCACTTACGGATCACAGAGTTTTAGAGCTAGAAATAGCAAGTTAAAATAAG GCTAGTCCGTTATCAACTTGAAAAAGTGGCACCGAGTCGGTGC (SEQ ID NO: 6) or GTATCACTGACGGAGCACAGAGTTTTAGAGCTAGAAATAGCAAGTTAAAATAAG GCTAGTCCGTTATCAACTTGAAAAAGTGGCACCGAGTCGGTGC (SEQ ID NO: 7). In certain embodiments, the gRNA comprises the sequence GTATCACTTACGGATCACAGAGTTTTAGAGCTAGAAATAGCAAGTTAAAATAAG GCTAGTCCGTTATCAACTTGAAAAAGTGGCACCGAGTCGGTGC (SEQ ID NO: 6). In certain embodiments, the gRNA comprises the sequence GTATCACTGACGGAGCACAGAGTTTTAGAGCTAGAAATAGCAAGTTAAAATAAG GCTAGTCCGTTATCAACTTGAAAAAGTGGCACCGAGTCGGTGC (SEQ ID NO: 7).

Additional sequences of suitable gRNAs for targeting a base editor to Tpp1 within the scope of the present disclosure will be apparent to those of skill in the art. Such suitable guide RNA sequences typically comprise a spacer sequence that is complementary to a nucleic sequence within 50 nucleotides (e.g., within 45, 40, 35, 30, 25, 20, 15, or 10 nucleotides) upstream or downstream of the target nucleotide to be edited (e.g., a target mutation in a Tpp1 gene).

In general, a gRNA is any RNA sequence having sufficient complementarity with a target polynucleotide sequence (e.g., Tpp1) to hybridize with the target sequence and direct sequence-specific binding of a napDNAbp (e.g., Cas9, which may be part of a base editor) to the target sequence. In some embodiments, the degree of complementarity between the spacer of a gRNA and its corresponding target sequence in Tpp1, when optimally aligned using a suitable alignment algorithm, is about or more than about 50%, 60%, 75%, 80%, 85%, 90%, 95%, 97.5%, 99%, or more (or the spacer and the corresponding target sequence comprise one, two, three, four, five, six, seven, eight, nine, or ten amino acid differences). In certain embodiments, the spacer of a gRNA is 100% complementary to its corresponding target sequence in Tpp1. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net).

The ability of a gRNA to direct sequence-specific binding of a base editor to a target sequence may also be assessed by any suitable assay. For example, a base editor and gRNA may be provided to a host cell (e.g., a cell of the CNS, such as a neuron or a glial cell) having the corresponding target sequence (e.g., Tpp1, or a portion thereof), such as by transfection with vectors encoding the base editor and gRNA or by transfection of a ribonucleoprotein (RNP) complex, followed by an assessment of preferential cleavage, nicking, or editing within the target sequence. Similarly, cleavage or editing of a target polynucleotide sequence may be evaluated in a test tube by providing the target sequence, base editor, and gRNA to be tested and a control gRNA different from the test gRNA, and comparing binding or rate of cleavage or editing at the target sequence between the test and control guide sequence reactions. Other assays are possible, and will be apparent to those skilled in the art.

In some embodiments, a gRNA is about 20, about 25, about 30, about 35, about 40, about 45, about 50, about 75, about 100, or more nucleotides in length. In some embodiments, a gRNA is about 50-150, about 60-140, about 70-130, about 80-120, or about 90-110 nucleotides in length. In some embodiments, the spacer sequence of a gRNA is about 10, about 11, about 12, about 13, about 14, about 15, about 16, about 17, about 18, about 19, about 20, about 21, about 22, about 23, about 24, about 25, about 26, about 27, about 28, about 29, or about 30 nucleotides in length.

In some embodiments, a gRNA comprises the structure 5′-[spacer sequence]-[backbone sequence]-3′. In some embodiments, a gRNA comprises an optional linker sequence. For example, the gRNAs provided herein may comprise an optional linker sequence between the spacer and the backbone sequence of the gRNA. In certain embodiments, the optional linker sequence is at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, or at least 50 nucleotides in length.

### Base Editors

The present disclosure provides complexes comprising any of the gRNAs provided herein and a base editor. In other aspects, the methods provided herein utilize any of the gRNAs provided herein and a base editor to edit Tpp1 (e.g., to correct an R208X mutation in a Tpp1 enzyme, where X is a premature stop codon). Any base editor known in the art may be used in the complexes, compositions, systems, and methods provided herein. In some embodiments, a base editor comprises a nucleic acid-programmable DNA binding protein (napDNAbp) and an adenosine deaminase.

In various embodiments, the base editors contemplated by the present disclosure comprise a napDNAbp. For example, base editors may include a napDNAbp domain having a wild type Cas9 sequence, including, for example, the canonical Streptococcus pyogenes Cas9 sequence of SEQ ID NO: 12, shown as follows.

| Description | Sequence |
|---|---|
| Wild type | MDKKYSIGLDIGTNSVGWAVITDEYKVPSKKFKVLGNTDRHSIK |
| Streptococcus | KNLIGALLFDSGETAEATRLKRTARRRYTRRKNRICYLQEIFSNE |
| pyogenes Cas9 | MAKVDDSFFHRLEESFLVEEDKKHERHPIFGNIVDEVAYHEKYP |
| (SpCas9) | TIYHLRKKLVDSTDKADLRLIYLALAHMIKFRGHFLIEGDLNPDN |
| | SDVDKLFIQLVQTYNQLFEENPINASGVDAKAILSARLSKSRRLE |
| | NLIAQLPGEKKNGLFGNLIALSLGLTPNFKSNFDLAEDAKLQLSK |
| | DTYDDDLDNLLAQIGDQYADLFLAAKNLSDAILLSDILRVNTEIT |
| | KAPLSASMIKRYDEHHQDLTLLKALVRQQLPEKYKEIFFDQSKN |
| | GYAGYIDGGASQEEFYKFIKPILEKMDGTEELLVKLNREDLLRK |
| | QRTFDNGSIPHQIHLGELHAILRRQEDFYPFLKDNREKIEKILTFRI |
| | PYYVGPLARGNSRFAWMTRKSEETITPWNFEEVVDKGASAQSFI |
| | ERMTNFDKNLPNEKVLPKHSLLYEYFTVYNELTKVKYVTEGMR |
| | KPAFLSGEQKKAIVDLLFKTNRKVTVKQLKEDYFKKIECFDSVEI |
| | SGVEDRFNASLGTYHDLLKIIKDKDFLDNEENEDILEDIVLTLTLF |
| | EDREMIEERLKTYAHLFDDKVMKQLKRRRYTGWGRLSRKLING |
| | IRDKQSGKTILDFLKSDGFANRNFMQLIHDDSLTFKEDIQKAQVS |
| | GQGDSLHEHIANLAGSPAIKKGILQTVKVVDELVKVMGRHKPE |
| | NIVIEMARENQTTQKGQKNSRERMKRIEEGIKELGSQILKEHPVE |
| | NTQLQNEKLYLYYLQNGRDMYVDQELDINRLSDYDVDHIVPQS |
| | FLKDDSIDNKVLTRSDKNRGKSDNVPSEEVVKKMKNYWRQLL |
| | NAKLITQRKFDNLTKAERGGLSELDKAGFIKRQLVETRQITKHV |
| | AQILDSRMNTKYDENDKLIREVKVITLKSKLVSDFRKDFQFYKV |
| | REINNYHHAHDAYLNAVVGTALIKKYPKLESEFVYGDYKVYDV |
| | RKMIAKSEQEIGKATAKYFFYSNIMNFFKTEITLANGEIRKRPLIE |
| | TNGETGEIVWDKGRDFATVRKVLSMPQVNIVKKTEVQTGGFSK |
| | ESILPKRNSDKLIARKKDWDPKKYGGFDSPTVAYSVLVVAKVE |
| | KGKSKKLKSVKELLGITIMERSSFEKNPIDFLEAKGYKEVKKDLII |
| | KLPKYSLFELENGRKRMLASAGELQKGNELALPSKYVNFLYLAS |
| | HYEKLKGSPEDNEQKQLFVEQHKHYLDEIIEQISEFSKRVILADA |
| | NLDKVLSAYNKHRDKPIREQAENIIHLFTLTNLGAPAAFKYFDTT |
| | IDRKRYTSTKEVLDATLIHQSITGLYETRIDLSQLGGD (SEQ ID |
| | NO: 12) |

In some embodiments, a base editor may include a napDNAbp domain having a modified Cas9 sequence, including, for example, nickase or nuclease-inactivated (dead) variants of Streptococcus pyogenes Cas9, shown as follows:

| | |
|---|---|
| Cas9 nickase | MDKKYSIGLDIGTNSVGWAVITDEYKVPSKKFKVLGNTDRHSIKK |
| Streptococcus | NLIGALLFDSGETAEATRLKRTARRRYTRRKNRICYLQEIFSNEMA |
| pyogenes Cas9 | KVDDSFFHRLEESFLVEEDKKHERHPIFGNIVDEVAYHEKYPTIYH |
| with H840A | LRKKLVDSTDKADLRLIYLALAHMIKFRGHFLIEGDLNPDNSDVD |
| | KLFIQLVQTYNQLFEENPINASGVDAKAILSARLSKSRRLENLIAQL |
| | PGEKKNGLFGNLIALSLGLTPNFKSNFDLAEDAKLQLSKDTYDDD |
| | LDNLLAQIGDQYADLFLAAKNLSDAILLSDILRVNTEITKAPLSAS |
| | MIKRYDEHHQDLTLLKALVRQQLPEKYKEIFFDQSKNGYAGYIDG |
| | GASQEEFYKFIKPILEKMDGTEELLVKLNREDLLRKQRTFDNGSIP |
| | HQIHLGELHAILRRQEDFYPFLKDNREKIEKILTFRIPYYVGPLARG |
| | NSRFAWMTRKSEETITPWNFEEVVDKGASAQSFIERMTNFDKNLP |
| | NEKVLPKHSLLYEYFTVYNELTKVKYVTEGMRKPAFLSGEQKKAI |
| | VDLLFKTNRKVTVKQLKEDYFKKIECFDSVEISGVEDRFNASLGT |
| | YHDLLKIIKDKDFLDNEENEDILEDIVLTLTLFEDREMIEERLKTYA |
| | HLFDDKVMKQLKRRRYTGWGRLSRKLINGIRDKQSGKTILDFLKS |
| | DGFANRNFMQLIHDDSLTFKEDIQKAQVSGQGDSLHEHIANLAGS |
| | PAIKKGILQTVKVVDELVKVMGRHKPENIVIEMARENQTTQKGQK |
| | NSRERMKRIEEGIKELGSQILKEHPVENTQLQNEKLYLYYLQNGR |
| | DMYVDQELDINRLSDYDVDAIVPQSFLKDDSIDNKVLTRSDKNRG |
| | KSDNVPSEEVVKKMKNYWRQLLNAKLITQRKFDNLTKAERGGLS |
| | ELDKAGFIKRQLVETRQITKHVAQILDSRMNTKYDENDKLIREVK |
| | VITLKSKLVSDFRKDFQFYKVREINNYHHAHDAYLNAVVGTALIK |
| | KYPKLESEFVYGDYKVYDVRKMIAKSEQEIGKATAKYFFYSNIMN |
| | FFKTEITLANGEIRKRPLIETNGETGEIVWDKGRDFATVRKVLSMP |
| | QVNIVKKTEVQTGGFSKESILPKRNSDKLIARKKDWDPKKYGGFD |
| | SPTVAYSVLVVAKVEKGKSKKLKSVKELLGITIMERSSFEKNPIDF |
| | LEAKGYKEVKKDLIIKLPKYSLFELENGRKRMLASAGELQKGNEL |
| | ALPSKYVNFLYLASHYEKLKGSPEDNEQKQLFVEQHKHYLDEIIE |
| | QISEFSKRVILADANLDKVLSAYNKHRDKPIREQAENIIHLFTLTNL |
| | GAPAAFKYFDTTIDRKRYTSTKEVLDATLIHQSITGLYETRIDLSQL |
| | GGD (SEQ ID NO: 13) |
| Cas9 nickase | MDKKYSIGLAIGTNSVGWAVITDEYKVPSKKFKVLGNTDRHSIKK |
| Streptococcus | NLIGALLFDSGETAEATRLKRTARRRYTRRKNRICYLQEIFSNEMA |
| pyogenes Cas9 | KVDDSFFHRLEESFLVEEDKKHERHPIFGNIVDEVAYHEKYPTIYH |
| with D10A | LRKKLVDSTDKADLRLIYLALAHMIKFRGHFLIEGDLNPDNSDVD |
| | KLFIQLVQTYNQLFEENPINASGVDAKAILSARLSKSRRLENLIAQL |
| | PGEKKNGLFGNLIALSLGLTPNFKSNFDLAEDAKLQLSKDTYDDD |
| | LDNLLAQIGDQYADLFLAAKNLSDAILLSDILRVNTEITKAPLSAS |
| | MIKRYDEHHQDLTLLKALVRQQLPEKYKEIFFDQSKNGYAGYIDG |
| | GASQEEFYKFIKPILEKMDGTEELLVKLNREDLLRKQRTFDNGSIP |
| | HQIHLGELHAILRRQEDFYPFLKDNREKIEKILTFRIPYYVGPLARG |
| | NSRFAWMTRKSEETITPWNFEEVVDKGASAQSFIERMTNFDKNLP |
| | NEKVLPKHSLLYEYFTVYNELTKVKYVTEGMRKPAFLSGEQKKAI |
| | VDLLFKTNRKVTVKQLKEDYFKKIECFDSVEISGVEDRFNASLGT |
| | YHDLLKIIKDKDFLDNEENEDILEDIVLTLTLFEDREMIEERLKTYA |
| | HLFDDKVMKQLKRRRYTGWGRLSRKLINGIRDKQSGKTILDFLKS |
| | DGFANRNFMQLIHDDSLTFKEDIQKAQVSGQGDSLHEHIANLAGS |
| | PAIKKGILQTVKVVDELVKVMGRHKPENIVIEMARENQTTQKGQK |
| | NSRERMKRIEEGIKELGSQILKEHPVENTQLQNEKLYLYYLQNGR |
| | DMYVDQELDINRLSDYDVDHIVPQSFLKDDSIDNKVLTRSDKNRG |
| | KSDNVPSEEVVKKMKNYWRQLLNAKLITQRKFDNLTKAERGGLS |
| | ELDKAGFIKRQLVETRQITKHVAQILDSRMNTKYDENDKLIREVK |
| | VITLKSKLVSDFRKDFQFYKVREINNYHHAHDAYLNAVVGTALIK |
| | KYPKLESEFVYGDYKVYDVRKMIAKSEQEIGKATAKYFFYSNIMN |
| | FFKTEITLANGEIRKRPLIETNGETGEIVWDKGRDFATVRKVLSMP |
| | QVNIVKKTEVQTGGFSKESILPKRNSDKLIARKKDWDPKKYGGFD |
| | SPTVAYSVLVVAKVEKGKSKKLKSVKELLGITIMERSSFEKNPIDF |
| | LEAKGYKEVKKDLIIKLPKYSLFELENGRKRMLASAGELQKGNEL |
| | ALPSKYVNFLYLASHYEKLKGSPEDNEQKQLFVEQHKHYLDEIIE |
| | QISEFSKRVILADANLDKVLSAYNKHRDKPIREQAENIIHLFTLTNL |
| | GAPAAFKYFDTTIDRKRYTSTKEVLDATLIHQSITGLYETRIDLSQL |
| | GGD (SEQ ID NO: 14) |
| Nuclease- | MDKKYSIGLDIGTNSVGWAVITDEYKVPSKKFKVLGNTDRHSIKK |
| inactivated Cas9 | NLIGALLFDSGETAEATRLKRTARRRYTRRKNRICYLQEIFSNEMA |
| (dCas9) | KVDDSFFHRLEESFLVEEDKKHERHPIFGNIVDEVAYHEKYPTIYH |
| Streptococcus | LRKKLVDSTDKADLRLIYLALAHMIKFRGHFLIEGDLNPDNSDVD |
| pyogenes Cas9 | KLFIQLVQTYNQLFEENPINASGVDAKAILSARLSKSRRLENLIAQL |
| with D10A and | PGEKKNGLFGNLIALSLGLTPNFKSNFDLAEDAKLQLSKDTYDDD |
| H840A | LDNLLAQIGDQYADLFLAAKNLSDAILLSDILRVNTEITKAPLSAS |
| | MIKRYDEHHQDLTLLKALVRQQLPEKYKEIFFDQSKNGYAGYIDG |
| | GASQEEFYKFIKPILEKMDGTEELLVKLNREDLLRKQRTFDNGSIP |
| | HQIHLGELHAILRRQEDFYPFLKDNREKIEKILTFRIPYYVGPLARG |
| | NSRFAWMTRKSEETITPWNFEEVVDKGASAQSFIERMTNFDKNLP |
| | NEKVLPKHSLLYEYFTVYNELTKVKYVTEGMRKPAFLSGEQKKAI |
| | VDLLFKTNRKVTVKQLKEDYFKKIECFDSVEISGVEDRFNASLGT |
| | YHDLLKIIKDKDFLDNEENEDILEDIVLTLTLFEDREMIEERLKTYA |
| | HLFDDKVMKQLKRRRYTGWGRLSRKLINGIRDKQSGKTILDFLKS |
| | DGFANRNFMQLIHDDSLTFKEDIQKAQVSGQGDSLHEHIANLAGS |
| | PAIKKGILQTVKVVDELVKVMGRHKPENIVIEMARENQTTQKGQK |
| | NSRERMKRIEEGIKELGSQILKEHPVENTQLQNEKLYLYYLQNGR |
| | DMYVDQELDINRLSDYDVDAIVPQSFLKDDSIDNKVLTRSDKNRG |
| | KSDNVPSEEVVKKMKNYWRQLLNAKLITQRKFDNLTKAERGGLS |
| | ELDKAGFIKRQLVETRQITKHVAQILDSRMNTKYDENDKLIREVK |
| | VITLKSKLVSDFRKDFQFYKVREINNYHHAHDAYLNAVVGTALIK |
| | KYPKLESEFVYGDYKVYDVRKMIAKSEQEIGKATAKYFFYSNIMN |
| | FFKTEITLANGEIRKRPLIETNGETGEIVWDKGRDFATVRKVLSMP |
| | QVNIVKKTEVQTGGFSKESILPKRNSDKLIARKKDWDPKKYGGFD |
| | SPTVAYSVLVVAKVEKGKSKKLKSVKELLGITIMERSSFEKNPIDF |
| | LEAKGYKEVKKDLIIKLPKYSLFELENGRKRMLASAGELQKGNEL |
| | ALPSKYVNFLYLASHYEKLKGSPEDNEQKQLFVEQHKHYLDEIIE |
| | QISEFSKRVILADANLDKVLSAYNKHRDKPIREQAENIIHLFTLTNL |
| | GAPAAFKYFDTTIDRKRYTSTKEVLDATLIHQSITGLYETRIDLSQL |
| | GGD (SEQ ID NO: 13) |

The base editors contemplated by the present disclosure may include any of the modified Cas9 sequences described above, or any variant thereof having at least 80%, at least 85%, at least 90%, at least 95%, at least 96%, at least 97%, at least 98%, or at least 99% sequence identity thereto. In some embodiments, a base editor comprises any of the following other wild type SpCas9 sequences, which may be modified with one or more of the mutations described herein (e.g., D10A and/or H840A) at corresponding amino acid positions:

Sequence

MDKKYSIGLDIGTNSVGWAVITDDYKVPSKKFKVLGNTDRHSIKKNLIGALLFGSG
ETAEATRLKRTARRRYTRRKNRICYLQEIFSNEMAKVDDSFFHRLEESFLVEEDKK
HERHPIFGNIVDEVAYHEKYPTIYHLRKKLADSTDKADLRLIYLALAHMIKFRGHFL
IEGDLNPDNSDVDKLFIQLVQIYNQLFEENPINASRVDAKAILSARLSKSRRLENLIA
QLPGEKRNGLFGNLIALSLGLTPNFKSNFDLAEDAKLQLSKDTYDDDLDNLLAQIG
DQYADLFLAAKNLSDAILLSDILRVNSEITKAPLSASMIKRYDEHHQDLTLLKALVR
QQLPEKYKEIFFDQSKNGYAGYIDGGASQEEFYKFIKPILEKMDGTEELLVKLNRED
LLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHS
LLYEYFTVYNELTKVKYVTEGMRKPAFLSGEQKKAIVDLLFKTNRKVTVKQLKED
YFKKIECFDSVEISGVEDRFNASLGAYHDLLKIIKDKDFLDNEENEDILEDIVLTLTLF
EDRGMIEERLKTYAHLFDDKVMKQLKRRRYTGWGRLSRKLINGIRDKQSGKTILD
FLKSDGFANRNFMQLIHDDSLTFKEDIQKAQVSGQGHSLHEQIANLAGSPAIKKGIL
QTVKIVDELVKVMGHKPENIVIEMARENQTTQKGQKNSRERMKRIEEGIKELGSQI
LKEHPVENTQLQNEKLYLYYLQNGRDMYVDQELDINRLSDYDVDHIVPQSFIKDD
SIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKL
VSDFRKDFQFYKVREINNYHHAHDAYLNAVVGTALIKKYPKLESEFVYGDYKVYD
VRKMIAKSEQEIGKATAKYFFYSNIMNFFKTEITLANGEIRKRPLIETNGETGEIVWD
KGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGY
KEVKKDLIIKLPKYSLFELENGRKRMLASAGELQKGNELALPSKYVNFLYLASHYE
KLKGSPEDNEQKQLFVEQHKHYLDEIIEQISEFSKRVILADANLDKVLSAYNKHRD
KPIREQAENIIHLFTLTNLGAPAAFKYFDTTIDRKRYTSTKEVLDATLIHQSITGLYET
RIDLSQLGGD (SEQ ID NO: 15)
MDKKYSIGLDIGTNSVGWAVITDEYKVPSKKFKVLGNTDRHSIKKNLIGALLFDSG
ETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFL
IEGDLNPDNSDVDKLFIQLVQTYNQLFEENPINASGVDAKAILSARLSKSRRLENLIA
QLPGEKKNGLFGNLIALSLGLTPNFKSNFDLAEDAKLQLSKDTYDDDLDNLLAQIG
DQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNRED
LLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHS
LLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLF
EDREMIEERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPAIKKGILQ
TVKVVDELVKVMGRHKPENIVIEMARENQTTQKGQKNSRERMKRIEEGIKELGSQI
LKEHPVENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKL
VSDFRKDFQFYKVREINNYHHAHDAYLNAVVGTALIKKYPKLESEFVYGDYKVYD
VRKMIAKSEQEIGKATAKYFFYSNIMNFFKTEITLANGEIRKRPLIETNGETGEIVWD
KGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGY
KEVKKDLIIKLPKYSLFELENGRKRMLASAGELQKGNELALPSKYVNFLYLASHYE
KLKGSPEDNEQKQLFVEQHKHYLDEIIEQISEFSKRVILADANLDKVLSAYNKHRD
KPIREQAENIIHLFTLTNLGAPAAFKYFDTTIDRKRYTSTKEVLDATLIHQSITGLYET
RIDLSQLGGDGSPKKKRKVSSDYKDHDGDYKDHDIDYKDDDDKAAG (SEQ ID
NO: 16)

In some embodiments, the Cas9 protein included in a base editor can be a wild type Cas9 ortholog from another bacterial species different from the canonical Cas9 from S. pyogenes. For example, modified versions of the following Cas9 orthologs can be used in connection with the base editors described in this specification by making mutations at positions corresponding to D10A and/or H840A or any other amino acids of interest in wild type SpCas9. In addition, any variant Cas9 orthologs having at least 80%, at least 85%, at least 90%, at least 95%, at least 96%, at least 97%, at least 98%, or at least 99% sequence identity to any of the below orthologs may also be used with the base editors.

| Description | Sequence |
|---|---|
| LfCas9 | MKEYHIGLDIGTSSIGWAVTDSQFKLMRIKGKTAIGVRLFEEGKT |
| Lactobacillus | AAERRTFRTTRRRLKRRKWRLHYLDEIFAPHLQEVDENFLRRLK |
| fermentum | QSNIHPEDPTKNQAFIGKLLFPDLLKKNERGYPTLIKMRDELPVEQ |
| Cas9 | RAHYPVMNIYKLREAMINEDRQFDLREVYLAVHHIVKYRGHFLN |
| | NASVDKFKVGRIDFDKSFNVLNEAYEELQNGEGSFTIEPSKVEKI |
| | GQLLLDTKMRKLDRQKAVAKLLEVKVADKEETKRNKQIATAMS |
| | KLVLGYKADFATVAMANGNEWKIDLSSETSEDEIEKFREELSDA |
| | QNDILTEITSLFSQIMLNEIVPNGMSISESMMDRYWTHERQLAEV |
| | KEYLATQPASARKEFDQVYNKYIGQAPKERGFDLEKGLKKILSK |
| | KENWKEIDELLKAGDFLPKQRTSANGVIPHQMHQQELDRIIEKQA |
| | KYYPWLATENPATGERDRHQAKYELDQLVSFRIPYYVGPLVTPE |
| | VQKATSGAKFAWAKRKEDGEITPWNLWDKIDRAESAEAFIKRM |
| | TVKDTYLLNEDVLPANSLLYQKYNVLNELNNVRVNGRRLSVGIK |
| | QDIYTELFKKKKTVKASDVASLVMAKTRGVNKPSVEGLSDPKKF |
| | NSNLATYLDLKSIVGDKVDDNRYQTDLENIIEWRSVFEDGEIFAD |
| | KLTEVEWLTDEQRSALVKKRYKGWGRLSKKLLTGIVDENGQRII |
| | DLMWNTDQNFKEIVDQPVFKEQIDQLNQKAITNDGMTLRERVES |
| | VLDDAYTSPQNKKAIWQVVRVVEDIVKAVGNAPKSISIEFARNE |
| | GNKGEITRSRRTQLQKLFEDQAHELVKDTSLTEELEKAPDLSDRY |
| | YFYFTQGGKDMYTGDPINFDEISTKYDIDHILPQSFVKDNSLDNR |
| | VLTSRKENNKKSDQVPAKLYAAKMKPYWNQLLKQGLITQRKFE |
| | NLTKDVDQNIKYRSLGFVKRQLVETRQVIKLTANILGSMYQEAG |
| | TEIIETRAGLTKQLREEFDLPKVREVNDYHHAVDAYLTTFAGQYL |
| | NRRYPKLRSFFVYGEYMKFKHGSDLKLRNFNFFHELMEGDKSQG |
| | KVVDQQTGELITTRDEVAKSFDRLLNMKYMLVSKEVHDRSDQL |
| | YGATIVTAKESGKLTSPIEIKKNRLVDLYGAYTNGTSAFMTIIKFT |
| | GNKPKYKVIGIPTTSAASLKRAGKPGSESYNQELHRIIKSNPKVKK |
| | GFEIVVPHVSYGQLIVDGDCKFTLASPTVQHPATQLVLSKKSLETI |
| | SSGYKILKDKPAIANERLIRVFDEVVGQMNRYFTIFDQRSNRQKV |
| | ADARDKFLSLPTESKYEGAKKVQVGKTEVITNLLMGLHANATQ |
| | GDLKVLGLATFGFFQSTTGLSLSEDTMIVYQSPTGLFERRICLKDI |
| | (SEQ ID NO: 17) |
| SaCas9 | MDKKYSIGLDIGTNSVGWAVITDEYKVPSKKFKVLGNTDRHSIK |
| Staphylococcus | KNLIGALLFDSGETAEATRLKRTARRRYTRRKNRICYLQEIFSNE |
| aureus | MAKVDDSFFHRLEESFLVEEDKKHERHPIFGNIVDEVAYHEKYPT |
| Cas9 | IYHLRKKLVDSTDKADLRLIYLALAHMIKFRGHFLIEGDLNPDNS |
| | DVDKLFIQLVQTYNQLFEENPINASGVDAKAILSARLSKSRRLEN |
| | LIAQLPGEKKNGLFGNLIALSLGLTPNFKSNFDLAEDAKLQLSKD |
| | TYDDDLDNLLAQIGDQYADLFLAAKNLSDAILLSDILRVNTEITK |
| | APLSASMIKRYDEHHQDLTLLKALVRQQLPEKYKEIFFDQSKNG |
| | YAGYIDGGASQEEFYKFIKPILEKMDGTEELLVKLNREDLLRKQR |
| | TFDNGSIPHQIHLGELHAILRRQEDFYPFLKDNREKIEKILTFRIPY |
| | YVGPLARGNSRFAWMTRKSEETITPWNFEEVVDKGASAQSFIER |
| | MTNFDKNLPNEKVLPKHSLLYEYFTVYNELTKVKYVTEGMRKP |
| | AFLSGEQKKAIVDLLFKTNRKVTVKQLKEDYFKKIECFDSVEISG |
| | VEDRFNASLGTYHDLLKIIKDKDFLDNEENEDILEDIVLTLTLFED |
| | REMIEERLKTYAHLFDDKVMKQLKRRRYTGWGRLSRKLINGIRD |
| | KQSGKTILDFLKSDGFANRNFMQLIHDDSLTFKEDIQKAQVSGQG |
| | DSLHEHIANLAGSPAIKKGILQTVKVVDELVKVMGRHKPENIVIE |
| | MARENQTTQKGQKNSRERMKRIEEGIKELGSQILKEHPVENTQL |
| | QNEKLYLYYLQNGRDMYVDQELDINRLSDYDVDHIVPQSFLKD |
| | DSIDNKVLTRSDKNRGKSDNVPSEEVVKKMKNYWRQLLNAKLI |
| | TQRKFDNLTKAERGGLSELDKAGFIKRQLVETRQITKHVAQILDS |
| | RMNTKYDENDKLIREVKVITLKSKLVSDFRKDFQFYKVREINNY |
| | HHAHDAYLNAVVGTALIKKYPKLESEFVYGDYKVYDVRKMIAK |
| | SEQEIGKATAKYFFYSNIMNFFKTEITLANGEIRKRPLIETNGETGE |
| | IVWDKGRDFATVRKVLSMPQVNIVKKTEVQTGGFSKESILPKRNS |
| | DKLIARKKDWDPKKYGGFDSPTVAYSVLVVAKVEKGKSKKLKS |
| | VKELLGITIMERSSFEKNPIDFLEAKGYKEVKKDLIIKLPKYSLFEL |
| | ENGRKRMLASAGELQKGNELALPSKYVNFLYLASHYEKLKGSPE |
| | DNEQKQLFVEQHKHYLDEIIEQISEFSKRVILADANLDKVLSAYN |
| | KHRDKPIREQAENIIHLFTLTNLGAPAAFKYFDTTIDRKRYTSTKE |
| | VLDATLIHQSITGLYETRIDLSQLGGD (SEQ ID NO: 12) |
| SaCas9 | MGKRNYILGLDIGITSVGYGIIDYETRDVIDAGVRLFKEANVENN |
| Staphylococcus | EGRRSKRGARRLKRRRRHRIQRVKKLLFDYNLLTDHSELSGINPY |
| aureus | EARVKGLSQKLSEEEFSAALLHLAKRRGVHNVNEVEEDTGNELS |
| Cas9 | TKEQISRNSKALEEKYVAELQLERLKKDGEVRGSINRFKTSDYVK |
| | EAKQLLKVQKAYHQLDQSFIDTYIDLLETRRTYYEGPGEGSPFG |
| | WKDIKEWYEMLMGHCTYFPEELRSVKYAYNADLYNALNDLNN |
| | LVITRDENEKLEYYEKFQIIENVFKQKKKPTLKQIAKEILVNEEDI |
| | KGYRVTSTGKPEFTNLKVYHDIKDITARKEIIENAELLDQIAKILTI |
| | YQSSEDIQEELTNLNSELTQEEIEQISNLKGYTGTHNLSLKAINLIL |
| | DELWHTNDNQIAIFNRLKLVPKKVDLSQQKEIPTTLVDDFILSPVV |
| | KRSFIQSIKVINAIIKKYGLPNDIIIELAREKNSKDAQKMINEMQKR |
| | NRQTNERIEEIIRTTGKENAKYLIEKIKLHDMQEGKCLYSLEAIPLE |
| | DLLNNPFNYEVDHIIPRSVSFDNSFNNKVLVKQEENSKKGNRTPF |
| | QYLSSSDSKISYETFKKHILNLAKGKGRISKTKKEYLLEERDINRF |
| | SVQKDFINRNLVDTRYATRGLMNLLRSYFRVNNLDVKVKSINGG |
| | FTSFLRRKWKFKKERNKGYKHHAEDALIIANADFIFKEWKKLDK |
| | AKKVMENQMFEEKQAESMPEIETEQEYKEIFITPHQIKHIKDFKD |
| | YKYSHRVDKKPNRKLINDTLYSTRKDDKGNTLIVNNLNGLYDKD |
| | NDKLKKLINKSPEKLLMYHHDPQTYQKLKLIMEQYGDEKNPLYK |
| | YYEETGNYLTKYSKKDNGPVIKKIKYYGNKLNAHLDITDDYPNS |
| | RNKVVKLSLKPYRFDVYLDNGVYKFVTVKNLDVIKKENYYEVN |
| | SKCYEEAKKLKKISNQAEFIASFYKNDLIKINGELYRVIGVNNDLL |
| | NRIEVNMIDITYREYLENMNDKRPPHIIKTIASKTQSIKKYSTDILG |
| | NLYEVKSKKHPQIIKK (SEQ ID NO: 18) |
| StCas9 | MLFNKCIIISINLDFSNKEKCMTKPYSIGLDIGTNSVGWAVITDNY |
| Streptococcus | KVPSKKMKVLGNTSKKYIKKNLLGVLLFDSGITAEGRRLKRTAR |
| thermophilus | RRYTRRRNRILYLQEIFSTEMATLDDAFFQRLDDSFLVPDDKRDS |
| Cas9 | KYPIFGNLVEEKVYHDEFPTIYHLRKYLADSTKKADLRLVYLALA |
| | HMIKYRGHFLIEGEFNSKNNDIQKNFQDFLDTYNAIFESDLSLENS |
| | KQLEEIVKDKISKLEKKDRILKLFPGEKNSGIFSEFLKLIVGNQADF |
| | RKCFNLDEKASLHFSKESYDEDLETLLGYIGDDYSDVFLKAKKL |
| | YDAILLSGFLTVTDNETEAPLSSAMIKRYNEHKEDLALLKEYIRNI |
| | SLKTYNEVFKDDTKNGYAGYIDGKTNQEDFYVYLKNLLAEFEG |
| | ADYFLEKIDREDFLRKQRTFDNGSIPYQIHLQEMRAILDKQAKFY |
| | PFLAKNKERIEKILTFRIPYYVGPLARGNSDFAWSIRKRNEKITPW |
| | NFEDVIDKESSAEAFINRMTSFDLYLPEEKVLPKHSLLYETFNVYN |
| | ELTKVRFIAESMRDYQFLDSKQKKDIVRLYFKDKRKVTDKDIIEY |
| | LHAIYGYDGIELKGIEKQFNSSLSTYHDLLNIINDKEFLDDSSNEAI |
| | IEEIIHTLTIFEDREMIKQRLSKFENIFDKSVLKKLSRRHYTGWGKL |
| | SAKLINGIRDEKSGNTILDYLIDDGISNRNFMQLIHDDALSFKKKI |
| | QKAQIIGDEDKGNIKEVVKSLPGSPAIKKGILQSIKIVDELVKVMG |
| | GRKPESIVVEMARENQYTNQGKSNSQQRLKRLEKSLKELGSKILK |
| | ENIPAKLSKIDNNALQNDRLYLYYLQNGKDMYTGDDLDIDRLSN |
| | YDIDHIIPQAFLKDNSIDNKVLVSSASNRGKSDDFPSLEVVKKRKT |
| | FWYQLLKSKLISQRKFDNLTKAERGGLLPEDKAGFIQRQLVETRQ |
| | ITKHVARLLDEKFNNKKDENNRAVRTVKIITLKSTLVSQFRKDFE |
| | LYKVREINDFHHAHDAYLNAVIASALLKKYPKLEPEFVYGDYPK |
| | YNSFRERKSATEKVYFYSNIMNIFKKSISLADGRVIERPLIEVNEET |
| | GESVWNKESDLATVRRVLSYPQVNVVKKVEEQNHGLDRGKPKG |
| | LFNANLSSKPKPNSNENLVGAKEYLDPKKYGGYAGISNSFAVLV |
| | KGTIEKGAKKKITNVLEFQGISILDRINYRKDKLNFLLEKGYKDIE |
| | LIIELPKYSLFELSDGSRRMLASILSTNNKRGEIHKGNQIFLSQKFV |
| | KLLYHAKRISNTINENHRKYVENHKKEFEELFYYILEFNENYVGA |
| | KKNGKLLNSAFQSWQNHSIDELCSSFIGPTGSERKGLFELTSRGSA |
| | ADFEFLGVKIPRYRDYTPSSLLKDATLIHQSVTGLYETRIDLAKLG |
| | EG (SEQ ID NO: 19) |
| LcCas9 | MKIKNYNLALTPSTSAVGHVEVDDDLNILEPVHHQKAIGVAKFG |
| Lactobacillus | EGETAEARRLARSARRTTKRRANRINHYFNEIMKPEIDKVDPLMF |
| crispatus | DRIKQAGLSPLDERKEFRTVIFDRPNIASYYHNQFPTIWHLQKYL |
| Cas9 | MITDEKADIRLIYWALHSLLKHRGHFFNTTPMSQFKPGKLNLKD |
| | DMLALDDYNDLEGLSFAVANSPEIEKVIKDRSMHKKEKIAELKK |
| | LIVNDVPDKDLAKRNNKIITQIVNAIMGNSFHLNFIFDMDLDKLTS |
| | KAWSFKLDDPELDTKFDAISGSMTDNQIGIFETLQKIYSAISLLDIL |
| | NGSSNVVDAKNALYDKHKRDLNLYFKFLNTLPDEIAKTLKAGYT |
| | LYIGNRKKDLLAARKLLKVNVAKNFSQDDFYKLINKELKSIDKQ |
| | GLQTRFSEKVGELVAQNNFLPVQRSSDNVFIPYQLNAITENKILEN |
| | QGKYYDFLVKPNPAKKDRKNAPYELSQLMQFTIPYYVGPLVTPE |
| | EQVKSGIPKTSRFAWMVRKDNGAITPWNFYDKVDIEATADKFIK |
| | RSIAKDSYLLSELVLPKHSLLYEKYEVFNELSNVSLDGKKLSGGV |
| | KQILFNEVFKKTNKVNTSRILKALAKHNIPGSKITGLSNPEEFTSSL |
| | QTYNAWKKYFPNQIDNFAYQQDLEKMIEWSTVFEDHKILAKKL |
| | DEIEWLDDDQKKFVANTRLRGWGRLSKRLLTGLKDNYGKSIMQ |
| | RLETTKANFQQIVYKPEFREQIDKISQAAAKNQSLEDILANSYTSP |
| | SNRKAIRKTMSVVDEYIKLNHGKEPDKIFLMFQRSEQEKGKQTE |
| | ARSKQLNRILSQLKADKSANKLFSKQLADEFSNAIKKSKYKLND |
| | KQYFYFQQLGRDALTGEVIDYDELYKYTVLHIIPRSKLTDDSQNN |
| | KVLTKYKIVDGSVALKFGNSYSDALGMPIKAFWTELNRLKLIPK |
| | GKLLNLTTDFSTLNKYQRDGYIARQLVETQQIVKLLATIMQSRFK |
| | HTKIIEVRNSQVANIRYQFDYFRIKNLNEYYRGFDAYLAAVVGTY |
| | LYKVYPKARRLFVYGQYLKPKKTNQENQDMHLDSEKKSQGFNF |
| | LWNLLYGKQDQIFVNGTDVIAFNRKDLITKMNTVYNYKSQKISL |
| | AIDYHNGAMFKATLFPRNDRDTAKTRKLIPKKKDYDTDIYGGYT |
| | SNVDGYMLLAEIIKRDGNKQYGFYGVPSRLVSELDTLKKTRYTE |
| | YEEKLKEIIKPELGVDLKKIKKIKILKNKVPFNQVIIDKGSKFFITST |
| | SYRWNYRQLILSAESQQTLMDLVVDPDFSNHKARKDARKNADE |
| | RLIKVYEEILYQVKNYMPMFVELHRCYEKLVDAQKTFKSLKISD |
| | KAMVLNQILILLHSNATSPVLEKLGYHTRFTLGKKHNLISENAVL |
| | VTQSITGLKENHVSIKQML (SEQ ID NO: 20) |
| PdCas9 | MTNEKYSIGLDIGTSSIGFAVVNDNNRVIRVKGKNAIGVRLFDEG |
| Pedicoccus | KAAADRRSFRTTRRSFRTTRRRLSRRRWRLKLLREIFDAYITPVD |
| damnosus | EAFFIRLKESNLSPKDSKKQYSGDILFNDRSDKDFYEKYPTIYHLR |
| Cas9 | NALMTEHRKFDVREIYLAIHHIMKFRGHFLNATPANNFKVGRLN |
| | LEEKFEELNDIYQRVFPDESIEFRTDNLEQIKEVLLDNKRSRADRQ |
| | RTLVSDIYQSSEDKDIEKRNKAVATEILKASLGNKAKLNVITNVE |
| | VDKEAAKEWSITFDSESIDDDLAKIEGQMTDDGHEIIEVLRSLYSG |
| | ITLSAIVPENHTLSQSMVAKYDLHKDHLKLFKKLINGMTDTKKA |
| | KNLRAAYDGYIDGVKGKVLPQEDFYKQVQVNLDDSAEANEIQT |
| | YIDQDIFMPKQRTKANGSIPHQLQQQELDQIIENQKAYYPWLAEL |
| | NPNPDKKRQQLAKYKLDELVTFRVPYYVGPMITAKDQKNQSGA |
| | EFAWMIRKEPGNITPWNFDQKVDRMATANQFIKRMTTTDTYLLG |
| | EDVLPAQSLLYQKFEVLNELNKIRIDHKPISIEQKQQIFNDLFKQF |
| | KNVTIKHLQDYLVSQGQYSKRPLIEGLADEKRFNSSLSTYSDLCGI |
| | FGAKLVEENDRQEDLEKIIEWSTIFEDKKIYRAKLNDLTWLTDDQ |
| | KEKLATKRYQGWGRLSRKLLVGLKNSEHRNIMDILWITNENFMQ |
| | IQAEPDFAKLVTDANKGMLEKTDSQDVINDLYTSPQNKKAIRQIL |
| | LVVHDIQNAMHGQAPAKIHVEFARGEERNPRRSVQRQRQVEAA |
| | YEKVSNELVSAKVRQEFKEAINNKRDFKDRLFLYFMQGGIDIYTG |
| | KQLNIDQLSSYQIDHILPQAFVKDDSLTNRVLTNENQVKADSVPI |
| | DIFGKKMLSVWGRMKDQGLISKGKYRNLTMNPENISAHTENGFI |
| | NRQLVETRQVIKLAVNILADEYGDSTQIISVKADLSHQMREDFEL |
| | LKNRDVNDYHHAFDAYLAAFIGNYLLKRYPKLESYFVYGDFKKF |
| | TQKETKMRRFNFIYDLKHCDQVVNKETGEILWTKDEDIKYIRHLF |
| | AYKKILVSHEVREKRGALYNQTIYKAKDDKGSGQESKKLIRIKD |
| | DKETKIYGGYSGKSLAYMTIVQITKKNKVSYRVIGIPTLALARLN |
| | KLENDSTENNGELYKIIKPQFTHYKVDKKNGEIIETTDDFKIVVSK |
| | VRFQQLIDDAGQFFMLASDTYKNNAQQLVISNNALKAINNTNITD |
| | CPRDDLERLDNLRLDSAFDEIVKKMDKYFSAYDANNFREKIRNS |
| | NLIFYQLPVEDQWENNKITELGKRTVLTRILQGLHANATTTDMSI |
| | FKIKTPFGQLRQRSGISLSENAQLIYQSPTGLFERRVQLNKIK (SEQ |
| | ID NO: 21) |
| FnCas9 | MKKQKFSDYYLGFDIGTNSVGWCVTDLDYNVLRFNKKDMWGS |
| Fusobaterium | RLFEEAKTAAERRVQRNSRRRLKRRKWRLNLLEEIFSNEILKIDSN |
| nucleatum | FFRRLKESSLWLEDKSSKEKFTLFNDDNYKDYDFYKQYPTIFHLR |
| Cas9 | NELIKNPEKKDIRLVYLAIHSIFKSRGHFLFEGQNLKEIKNFETLYN |
| | NLIAFLEDNGINKIIDKNNIEKLEKIVCDSKKGLKDKEKEFKEIFNS |
| | DKQLVAIFKLSVGSSVSLNDLFDTDEYKKGEVEKEKISFREQIYED |
| | DKPIYYSILGEKIELLDIAKTFYDFMVLNNILADSQYISEAKVKLY |
| | EEHKKDLKNLKYIIRKYNKGNYDKLFKDKNENNYSAYIGLNKEK |
| | SKKEVIEKSRLKIDDLIKNIKGYLPKVEEIEEKDKAIFNKILNKIEL |
| | KTILPKQRISDNGTLPYQIHEAELEKILENQSKYYDFLNYEENGIIT |
| | KDKLLMTFKFRIPYYVGPLNSYHKDKGGNSWIVRKEEGKILPWN |
| | FEQKVDIEKSAEEFIKRMTNKCTYLNGEDVIPKDTFLYSEYVILNE |
| | LNKVQVNDEFLNEENKRKIIDELFKENKKVSEKKFKEYLLVKQIV |
| | DGTIELKGVKDSFNSNYISYIRFKDIFGEKLNLDIYKEISEKSILWK |
| | CLYGDDKKIFEKKIKNEYGDILTKDEIKKINTFKFNNWGRLSEKL |
| | LTGIEFINLETGECYSSVMDALRRTNYNLMELLSSKFTLQESINNE |
| | NKEMNEASYRDLIEESYVSPSLKRAIFQTLKIYEEIRKITGRVPKK |
| | VFIEMARGGDESMKNKKIPARQEQLKKLYDSCGNDIANFSIDIKE |
| | MKNSLISYDNNSLRQKKLYLYYLQFGKCMYTGREIDLDRLLQNN |
| | DTYDIDHIYPRSKVIKDDSFDNLVLVLKNENAEKSNEYPVKKEIQ |
| | EKMKSFWRFLKEKNFISDEKYKRLTGKDDFELRGFMARQLVNV |
| | RQTTKEVGKILQQIEPEIKIVYSKAEIASSFREMFDFIKVRELNDTH |
| | HAKDAYLNIVAGNVYNTKFTEKPYRYLQEIKENYDVKKIYNYDI |
| | KNAWDKENSLEIVKKNMEKNTVNITRFIKEKKGQLFDLNPIKKG |
| | ETSNEIISIKPKVYNGKDDKLNEKYGYYKSLNPAYFLYVEHKEKN |
| | KRIKSFERVNLVDVNNIKDEKSLVKYLIENKKLVEPRVIKKVYKR |
| | QVILINDYPYSIVTLDSNKLMDFENLKPLFLENKYEKILKNVIKFL |
| | EDNQGKSEENYKFIYLKKKDRYEKNETLESVKDRYNLEFNEMYD |
| | KFLEKLDSKDYKNYMNNKKYQELLDVKEKFIKLNLFDKAFTLKS |
| | FLDLFNRKTMADFSKVGLTKYLGKIQKISSNVLSKNELYLLEESV |
| | TGLFVKKIKL (SEQ ID NO: 22) |
| EcCas9 | RRKQRIQILQELLGEEVLKTDPGFFHRMKESRYVVEDKRTLDGK |
| Enterococcus | QVELPYALFVDKDYTDKEYYKQFPTINHLIVYLMTTSDTPDIRLV |
| cecorum Cas9 | YLALHYYMKNRGNFLHSGDINNVKDINDILEQLDNVLETFLDGW |
| | NLKLKSYVEDIKNIYNRDLGRGERKKAFVNTLGAKTKAEKAFCS |
| | LISGGSTNLAELFDDSSLKEIETPKIEFASSSLEDKIDGIQEALEDRF |
| | AVIEAAKRLYDWKTLTDILGDSSSLAEARVNSYQMHHEQLLELK |
| | SLVKEYLDRKVFQEVFVSLNVANNYPAYIGHTKINGKKKELEVK |
| | RTKRNDFYSYVKKQVIEPIKKKVSDEAVLTKLSEIESLIEVDKYLP |
| | LQVNSDNGVIPYQVKLNELTRIFDNLENRIPVLRENRDKIIKTFKF |
| | RIPYYVGSLNGVVKNGKCTNWMVRKEEGKIYPWNFEDKVDLEA |
| | SAEQFIRRMTNKCTYLVNEDVLPKYSLLYSKYLVLSELNNLRIDG |
| | RPLDVKIKQDIYENVFKKNRKVTLKKIKKYLLKEGIITDDDELSGL |
| | ADDVKSSLTAYRDFKEKLGHLDLSEAQMENIILNITLFGDDKKLL |
| | KKRLAALYPFIDDKSLNRIATLNYRDWGRLSERFLSGITSVDQET |
| | GELRTIIQCMYETQANLMQLLAEPYHFVEAIEKENPKVDLESISYR |
| | IVNDLYVSPAVKRQIWQTLLVIKDIKQVMKHDPERIFIEMAREKQ |
| | ESKKTKSRKQVLSEVYKKAKEYEHLFEKLNSLTEEQLRSKKIYLY |
| | FTQLGKCMYSGEPIDFENLVSANSNYDIDHIYPQSKTIDDSENNIV |
| | LVKKSLNAYKSNHYPIDKNIRDNEKVKTLWNTLVSKGLITKEKY |
| | ERLIRSTPFSDEELAGFIARQLVETRQSTKAVAEILSNWFPESEIVY |
| | SKAKNVSNFRQDFEILKVRELNDCHHAHDAYLNIVVGNAYHTKF |
| | TNSPYRFIKNKANQEYNLRKLLQKVNKIESNGVVAWVGQSENNP |
| | GTIATVKKVIRRNTVLISRMVKEVDGQLFDLTLMKKGKGQVPIKS |
| | SDERLTDISKYGGYNKATGAYFTFVKSKKRGKVVRSFEYVPLHL |
| | SKQFENNNELLKEYIEKDRGLTDVEILIPKVLINSLFRYNGSLVRIT |
| | GRGDTRLLLVHEQPLYVSNSFVQQLKSVSSYKLKKSENDNAKLT |
| | KTATEKLSNIDELYDGLLRKLDLPIYSYWFSSIKEYLVESRTKYIK |
| | LSIEEKALVIFEILHLFQSDAQVPNLKILGLSTKPSRIRIQKNLKDTD |
| | KMSIIHQSPSGIFEHEIELTSL (SEQ ID NO: 23) |
| AhCas9 | MQNGFLGITVSSEQVGWAVTNPKYELERASRKDLWGVRLFDKA |
| Anaerostipes | ETAEDRRMFRTNRRLNQRKKNRIHYLRDIFHEEVNQKDPNFFQQ |
| hadrus Cas9 | LDESNFCEDDRTVEFNFDTNLYKNQFPTVYHLRKYLMETKDKPD |
| | IRLVYLAFSKFMKNRGHFLYKGNLGEVMDFENSMKGFCESLEKF |
| | NIDFPTLSDEQVKEVRDILCDHKIAKTVKKKNIITITKVKSKTAKA |
| | WIGLFCGCSVPVKVLFQDIDEEIVTDPEKISFEDASYDDYIANIEK |
| | GVGIYYEAIVSAKMLFDWSILNEILGDHQLLSDAMIAEYNKHHD |
| | DLKRLQKIIKGTGSRELYQDIFINDVSGNYVCYVGHAKTMSSADQ |
| | KQFYTFLKNRLKNVNGISSEDAEWIDTEIKNGTLLPKQTKRDNSV |
| | IPHQLQLREFELILDNMQEMYPFLKENREKLLKIFNFVIPYYVGPL |
| | KGVVRKGESTNWMVPKKDGVIHPWNFDEMVDKEASAECFISRM |
| | TGNCSYLFNEKVLPKNSLLYETFEVLNELNPLKINGEPISVELKQR |
| | IYEQLFLTGKKVTKKSLTKYLIKNGYDKDIELSGIDNEFHSNLKSH |
| | IDFEDYDNLSDEEVEQIILRITVFEDKQLLKDYLNREFVKLSEDER |
| | KQICSLSYKGWGNLSEMLLNGITVTDSNGVEVSVMDMLWNTNL |
| | NLMQILSKKYGYKAEIEHYNKEHEKTIYNREDLMDYLNIPPAQR |
| | RKVNQLITIVKSLKKTYGVPNKIFFKISREHQDDPKRTSSRKEQLK |
| | YLYKSLKSEDEKHLMKELDELNDHELSNDKVYLYFLQKGRCIYS |
| | GKKLNLSRLRKSNYQNDIDYIYPLSAVNDRSMNNKVLTGIQENR |
| | ADKYTYFPVDSEIQKKMKGFWMELVLQGFMTKEKYFRLSREND |
| | FSKSELVSFIEREISDNQQSGRMIASVLQYYFPESKIVFVKEKLISSF |
| | KRDFHLISSYGHNHLQAAKDAYITIVVGNVYHTKFTMDPAIYFK |
| | NHKRKDYDLNRLFLENISRDGQIAWESGPYGSIQTVRKEYAQNHI |
| | AVTKRVVEVKGGLFKQMPLKKGHGEYPLKTNDPRFGNIAQYGG |
| | YTNVTGSYFVLVESMEKGKKRISLEYVPVYLHERLEDDPGHKLL |
| | KEYLVDHRKLNHPKILLAKVRKNSLLKIDGFYYRLNGRSGNALIL |
| | TNAVELIMDDWQTKTANKISGYMKRRAIDKKARVYQNEFHIQEL |
| | EQLYDFYLDKLKNGVYKNRKNNQAELIHNEKEQFMELKTEDQC |
| | VLLTEIKKLFVCSPMQADLTLIGGSKHTGMIAMSSNVTKADFAVI |
| | AEDPLGLRNKVIYSHKGEK (SEQ ID NO: 24) |
| KvCas9 | MSQNNNKIYNIGLDIGDASVGWAVVDEHYNLLKRHGKHMWGS |
| Kandleria | RLFTQANTAVERRSSRSTRRRYNKRRERIRLLREIMEDMVLDVDP |
| vitulina Cas9 | TFFIRLANVSFLDQEDKKDYLKENYHSNYNLFIDKDFNDKTYYD |
| | KYPTIYHLRKHLCESKEKEDPRLIYLALHHIVKYRGNFLYEGQKF |
| | SMDVSNIEDKMIDVLRQFNEINLFEYVEDRKKIDEVLNVLKEPLS |
| | KKHKAEKAFALFDTTKDNKAAYKELCAALAGNKFNVTKMLKE |
| | AELHDEDEKDISFKFSDATFDDAFVEKQPLLGDCVEFIDLLHDIYS |
| | WVELQNILGSAHTSEPSISAAMIQRYEDHKNDLKLLKDVIRKYLP |
| | KKYFEVFRDEKSKKNNYCNYINHPSKTPVDEFYKYIKKLIEKIDD |
| | PDVKTILNKIELESFMLKQNSRTNGAVPYQMQLDELNKILENQSV |
| | YYSDLKDNEDKIRSILTFRIPYYFGPLNITKDRQFDWIIKKEGKEN |
| | ERILPWNANEIVDVDKTADEFIKRMRNFCTYFPDEPVMAKNSLT |
| | VSKYEVLNEINKLRINDHLIKRDMKDKMLHTLFMDHKSISANAM |
| | KKWLVKNQYFSNTDDIKIEGFQKENACSTSLTPWIDFTKIFGKINE |
| | SNYDFIEKIIYDVTVFEDKKILRRRLKKEYDLDEEKIKKILKLKYS |
| | GWSRLSKKLLSGIKTKYKDSTRTPETVLEVMERTNMNLMQVIND |
| | EKLGFKKTIDDANSTSVSGKFSYAEVQELAGSPAIKRGIWQALLI |
| | VDEIKKIMKHEPAHVYIEFARNEDEKERKDSFVNQMLKLYKDYD |
| | FEDETEKEANKHLKGEDAKSKIRSERLKLYYTQMGKCMYTGKSL |
| | DIDRLDTYQVDHIVPQSLLKDDSIDNKVLVLSSENQRKLDDLVIPS |
| | SIRNKMYGFWEKLFNNKIISPKKFYSLIKTEFNEKDQERFINRQIVE |
| | TRQITKHVAQIIDNHYENTKVVTVRADLSHQFRERYHIYKNRDIN |
| | DFHHAHDAYIATILGTYIGHRFESLDAKYIYGEYKRIFRNQKNKG |
| | KEMKKNNDGFILNSMRNIYADKDTGEIVWDPNYIDRIKKCFYYK |
| | DCFVTKKLEENNGTFFNVTVLPNDTNSDKDNTLATVPVNKYRSN |
| | VNKYGGFSGVNSFIVAIKGKKKKGKKVIEVNKLTGIPLMYKNAD |
| | EEIKINYLKQAEDLEEVQIGKEILKNQLIEKDGGLYYIVAPTEIINA |
| | KQLILNESQTKLVCEIYKAMKYKNYDNLDSEKIIDLYRLLINKME |
| | LYYPEYRKQLVKKFEDRYEQLKVISIEEKCNIIKQILATLHCNSSIG |
| | KIMYSDFKISTTIGRLNGRTISLDDISFIAESPTGMYSKKYKL (SEQ |
| | ID NO: 25) |
| EfCas9 | MRLFEEGHTAEDRRLKRTARRRISRRRNRLRYLQAFFEEAMTDL |
| Enterococcus | DENFFARLQESFLVPEDKKWHRHPIFAKLEDEVAYHETYPTIYHL |
| faecalis Cas9 | RKKLADSSEQADLRLIYLALAHIVKYRGHFLIEGKLSTENTSVKD |
| | QFQQFMVIYNQTFVNGESRLVSAPLPESVLIEEELTEKASRTKKSE |
| | KVLQQFPQEKANGLFGQFLKLMVGNKADFKKVFGLEEEAKITYA |
| | SESYEEDLEGILAKVGDEYSDVFLAAKNVYDAVELSTILADSDKK |
| | SHAKLSSSMIVRFTEHQEDLKKFKRFIRENCPDEYDNLFKNEQKD |
| | GYAGYIAHAGKVSQLKFYQYVKKIIQDIAGAEYFLEKIAQENFLR |
| | KQRTFDNGVIPHQIHLAELQAIIHRQAAYYPFLKENQEKIEQLVTF |
| | RIPYYVGPLSKGDASTFAWLKRQSEEPIRPWNLQETVDLDQSATA |
| | FIERMTNFDTYLPSEKVLPKHSLLYEKFMVFNELTKISYTDDRGIK |
| | ANFSGKEKEKIFDYLFKTRRKVKKKDIIQFYRNEYNTEIVTLSGLE |
| | EDQFNASFSTYQDLLKCGLTRAELDHPDNAEKLEDIIKILTIFEDR |
| | QRIRTQLSTFKGQFSAEVLKKLERKHYTGWGRLSKKLINGIYDKE |
| | SGKTILDYLVKDDGVSKHYNRNFMQLINDSQLSFKNAIQKAQSSE |
| | HEETLSETVNELAGSPAIKKGIYQSLKIVDELVAIMGYAPKRIVVE |
| | MARENQTTSTGKRRSIQRLKIVEKAMAEIGSNLLKEQPTTNEQLR |
| | DTRLFLYYMQNGKDMYTGDELSLHRLSHYDIDHIIPQSFMKDDS |
| | LDNLVLVGSTENRGKSDDVPSKEVVKDMKAYWEKLYAAGLISQ |
| | RKFQRLTKGEQGGLTLEDKAHFIQRQLVETRQITKNVAGILDQRY |
| | NAKSKEKKVQIITLKASLTSQFRSIFGLYKVREVNDYHHGQDAYL |
| | NCVVATTLLKVYPNLAPEFVYGEYPKFQTFKENKATAKAIIYTNL |
| | LRFFTEDEPRFTKDGEILWSNSYLKTIKKELNYHQMNIVKKVEVQ |
| | KGGFSKESIKPKGPSNKLIPVKNGLDPQKYGGFDSPVVAYTVLFT |
| | HEKGKKPLIKQEILGITIMEKTRFEQNPILFLEEKGFLRPRVLMKLP |
| | KYTLYEFPEGRRRLLASAKEAQKGNQMVLPEHLLTLLYHAKQCL |
| | LPNQSESLAYVEQHQPEFQEILERVVDFAEVHTLAKSKVQQIVKL |
| | FEANQTADVKEIAASFIQLMQFNAMGAPSTFKFFQKDIERARYTSI |
| | KEIFDATIIYQSPTGLYETRRKVVD (SEQ ID NO: 26) |
| Staphylococcus | KRNYILGLDIGITSVGYGIIDYETRDVIDAGVRLFKEANVENNEGR |
| aureus | RSKRGARRLKRRRRHRIQRVKKLLFDYNLLTDHSELSGINPYEAR |
| Cas9 | VKGLSQKLSEEEFSAALLHLAKRRGVHNVNEVEEDTGNELSTKE |
| | QISRNSKALEEKYVAELQLERLKKDGEVRGSINRFKTSDYVKEAK |
| | QLLKVQKAYHQLDQSFIDTYIDLLETRRTYYEGPGEGSPFGWKDI |
| | KEWYEMLMGHCTYFPEELRSVKYAYNADLYNALNDLNNLVITR |
| | DENEKLEYYEKFQIIENVFKQKKKPTLKQIAKEILVNEEDIKGYRV |
| | TSTGKPEFTNLKVYHDIKDITARKEIIENAELLDQIAKILTIYQSSE |
| | DIQEELTNLNSELTQEEIEQISNLKGYTGTHNLSLKAINLILDELW |
| | HTNDNQIAIFNRLKLVPKKVDLSQQKEIPTTLVDDFILSPVVKRSFI |
| | QSIKVINAIIKKYGLPNDIIIELAREKNSKDAQKMINEMQKRNRQT |
| | NERIEEIIRTTGKENAKYLIEKIKLHDMQEGKCLYSLEAIPLEDLLN |
| | NPFNYEVDHIIPRSVSFDNSFNNKVLVKQEENSKKGNRTPFQYLS |
| | SSDSKISYETFKKHILNLAKGKGRISKTKKEYLLEERDINRFSVQK |
| | DFINRNLVDTRYATRGLMNLLRSYFRVNNLDVKVKSINGGFTSFL |
| | RRKWKFKKERNKGYKHHAEDALIIANADFIFKEWKKLDKAKKV |
| | MENQMFEEKQAESMPEIETEQEYKEIFITPHQIKHIKDFKDYKYSH |
| | RVDKKPNRELINDTLYSTRKDDKGNTLIVNNLNGLYDKDNDKLK |
| | KLINKSPEKLLMYHHDPQTYQKLKLIMEQYGDEKNPLYKYYEET |
| | GNYLTKYSKKDNGPVIKKIKYYGNKLNAHLDITDDYPNSRNKVV |
| | KLSLKPYRFDVYLDNGVYKFVTVKNLDVIKKENYYEVNSKCYEE |
| | AKKLKKISNQAEFIASFYNNDLIKINGELYRVIGVNNDLLNRIEVN |
| | MIDITYREYLENMNDKRPPRIIKTIASKTQSIKKYSTDILGNLYEVK |
| | SKKHPQIIKKG (SEQ ID NO: 27) |
| Geobacillus | MKYKIGLDIGITSIGWAVINLDIPRIEDLGVRIFDRAENPKTGESLA |
| thermo- | LPRRLARSARRRLRRRKHRLERIRRLFVREGILTKEELNKLFEKKH |
| denitrificans | EIDVWQLRVEALDRKLNNDELARILLHLAKRRGFRSNRKSERTN |
| Cas9 | KENSTMLKHIEENQSILSSYRTVAEMVVKDPKFSLHKRNKEDNY |
| (“GeoCas9”) | TNTVARDDLEREIKLIFAKQREYGNIVCTEAFEHEYISIWASQRPF |
| | ASKDDIEKKVGFCTFEPKEKRAPKATYTFQSFTVWEHINKLRLVS |
| | PGGIRALTDDERRLIYKQAFHKNKITFHDVRTLLNLPDDTRFKGL |
| | LYDRNTTLKENEKVRFLELGYHKIRKAIDSVYGKGAAKSFRPID |
| | FDTFGYALTMFKDDTDIRSYLRNEYEQNGKRMENLADKVYDEE |
| | LIEELLNLSFSKFGHLSLKALRNILPYMEQGEVYSTACERAGYTFT |
| | GPKKKQKTVLLPNIPPIANPVVMRALTQARKVVNAIIKKYGSPVSI |
| | HIELARELSQSFDERRKMQKEQEGNRKKNETAIRQLVEYGLTLNP |
| | TGLDIVKFKLWSEQNGKCAYSLQPIEIERLLEPGYTEVDHVIPYSR |
| | SLDDSYTNKVLVLTKENREKGNRTPAEYLGLGSERWQQFETFVL |
| | TNKQFSKKKRDRLLRLHYDENEENEFKNRNLNDTRYISRFLANFI |
| | REHLKFADSDDKQKVYTVNGRITAHLRSRWNFNKNREESNLHH |
| | AVDAAIVACTTPSDIARVTAFYQRREQNKELSKKTDPQFPQPWPH |
| | FADELQARLSKNPKESIKALNLGNYDNEKLESLQPVFVSRMPKRS |
| | ITGAAHQETLRRYIGIDERSGKIQTVVKKKLSEIQLDKTGHFPMY |
| | GKESDPRTYEAIRQRLLEHNNDPKKAFQEPLYKPKKNGELGPIIRT |
| | IKIIDTTNQVIPLNDGKTVAYNSNIVRVDVFEKDGKYYCVPIYTID |
| | MMKGILPNKAIEPNKPYSEWKEMTEDYTFRFSLYPNDLIRIEFPRE |
| | KTIKTAVGEEIKIKDLFAYYQTIDSSNGGLSLVSHDNNFSLRSIGSR |
| | TLKRFEKYQVDVLGNIYKVRGEKRVGVASSSHSKAGETIRPL |
| | (SEQ ID NO: 28) |
| ScCas9 | MEKKYSIGLDIGTNSVGWAVITDDYKVPSKKFKVLGNTNRKSIK |
| S. canis Cas9 | KNLMGALLFDSGETAEATRLKRTARRRYTRRKNRIRYLQEIFANE |
| | MAKLDDSFFQRLEESFLVEEDKKNERHPIFGNLADEVAYHRNYP |
| | TIYHLRKKLADSPEKADLRLIYLALAHIIKFRGHFLIEGKLNAENS |
| | DVAKLFYQLIQTYNQLFEESPLDEIEVDAKGILSARLSKSKRLEKL |
| | IAVFPNEKKNGLFGNIIALALGLTPNFKSNFDLTEDAKLQLSKDTY |
| | DDDLDELLGQIGDQYADLFSAAKNLSDAILLSDILRSNSEVTKAP |
| | LSASMVKRYDEHHQDLALLKTLVRQQFPEKYAEIFKDDTKNGY |
| | AGYVGIGIKHRKRTTKLATQEEFYKFIKPILEKMDGAEELLAKLN |
| | RDDLLRKQRTFDNGSIPHQIHLKELHAILRRQEEFYPFLKENREKI |
| | EKILTFRIPYYVGPLARGNSRFAWLTRKSEEAITPWNFEEVVDKG |
| | ASAQSFIERMTNFDEQLPNKKVLPKHSLLYEYFTVYNELTKVKY |
| | VTERMRKPEFLSGEQKKAIVDLLFKTNRKVTVKQLKEDYFKKIE |
| | CFDSVEIIGVEDRFNASLGTYHDLLKIIKDKDFLDNEENEDILEDIV |
| | LTLTLFEDREMIEERLKTYAHLFDDKVMKQLKRRHYTGWGRLSR |
| | KMINGIRDKQSGKTILDFLKSDGFSNRNFMQLIHDDSLTFKEEIEK |
| | AQVSGQGDSLHEQIADLAGSPAIKKGILQTVKIVDELVKVMGHK |
| | PENIVIEMARENQTTTKGLQQSRERKKRIEEGIKELESQILKENPV |
| | ENTQLQNEKLYLYYLQNGRDMYVDQELDINRLSDYDVDHIVPQS |
| | FIKDDSIDNKVLTRSVENRGKSDNVPSEEVVKKMKNYWRQLLNA |
| | KLITQRKFDNLTKAERGGLSEADKAGFIKRQLVETRQITKHVARI |
| | LDSRMNTKRDKNDKPIREVKVITLKSKLVSDFRKDFQLYKVRDIN |
| | NYHHAHDAYLNAVVGTALIKKYPKLESEFVYGDYKVYDVRKMI |
| | AKSEQEIGKATAKRFFYSNIMNFFKTEVKLANGEIRKRPLIETNGE |
| | TGEVVWNKEKDFATVRKVLAMPQVNIVKKTEVQTGGFSKESILS |
| | KRESAKLIPRKKGWDTRKYGGFGSPTVAYSILVVAKVEKGKAKK |
| | LKSVKVLVGITIMEKGSYEKDPIGFLEAKGYKDIKKELIFKLPKYS |
| | LFELENGRRRMLASATELQKANELVLPQHLVRLLYYTQNISATT |
| | GSNNLGYIEQHREEFKEIFEKIIDFSEKYILKNKVNSNLKSSFDEQF |
| | AVSDSILLSNSFVSLLKYTSFGASGGFTFLDLDVKQGRLRYQTVT |
| | EVLDATLIYQSITGLYETRTDLSQLGGD (SEQ ID NO: 29) |

Additional suitable napDNAbp sequences that can be used in base editors will be apparent to those of skill in the art based on this disclosure, and such Cas9 proteins include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; which is incorporated herein by reference. Additional exemplary Cas variants and homologs include, but are not limited to, Cas9 (e.g., dCas9 and nCas9), Cpf1, CasX, CasY, C2cl, C2c2, C2c3, GeoCas9, CjCas9, Cas12a, Cas12b, Cas12g, Cas12h, Cas12i, Cas13b, Cas13c, Cas13d, Cas14, Csn2, xCas9, SpCas9-NG, Nme2Cas9, circularly permuted Cas9, Argonaute (Ago), Cas9-KKH, SmacCas9, Spy-macCas9, SpCas9-VRQR, SpCas9-NRRH, SpaCas9-NRTH, SpCas9-NRCH, LbCas12a, AsCas12a, CeCas12a, MbCas12a, Cas3, CasΦ, and circularly permuted Cas9 domains such as CP1012, CP1028, CP1041, CP1249, and CP1300, and variants and homologs thereof.

In various embodiments, the base editors contemplated for use in the present disclosure comprise a deaminase domain. In some embodiments, a base editor converts an A to a G. In some embodiments, the base editor comprises an adenosine deaminase. In some embodiments, the deaminase is an E. coli TadA (ecTadA) deaminase, or a variant thereof. Adenosine deaminases are described, for example, in International PCT Application Publication No. WO2018/027078, which is incorporated herein by reference. In some embodiments, an adenosine deaminase comprises any of the following amino acid sequences, or an amino acid sequence at least 80%, at least 85%, at least 90%, at least 95%, at least 96%, at least 97%, at least 98%, or at least 99% identical to any of the following amino acid sequences:

ecTadA
(SEQ ID NO: 30)
SEVEFSHEYWMRHALTLAKRAWDEREVPVGAVLVHNNRVIGEGWNRPIGRHDPTA
HAEIMALRQGGLVMQNYRLIDATLYVTLEPCVMCAGAMIHSRIGRVVFGARDAKTG
AAGSLMDVLHHPGMNHRVEITEGILADECAALLSDFFRMRRQEIKAQKKAQSSTD
ecTadA (D108N)
(SEQ ID NO: 31)
SEVEFSHEYWMRHALTLAKRAWDEREVPVGAVLVHNNRVIGEGWNRPIGRHDPTA
HAEIMALRQGGLVMQNYRLIDATLYVTLEPCVMCAGAMIHSRIGRVVFGARNAKTG
AAGSLMDVLHHPGMNHRVEITEGILADECAALLSDFFRMRRQEIKAQKKAQSSTD
ecTadA (D108G)
(SEQ ID NO: 32)
SEVEFSHEYWMRHALTLAKRAWDEREVPVGAVLVHNNRVIGEGWNRPIGRHDPTA
HAEIMALRQGGLVMQNYRLIDATLYVTLEPCVMCAGAMIHSRIGRVVFGARGAKT
GAAGSLMDVLHHPGMNHRVEITEGILADECAALLSDFFRMRRQEIKAQKKAQSSTD
ecTadA (D108V)
(SEQ ID NO: 33)
SEVEFSHEYWMRHALTLAKRAWDEREVPVGAVLVHNNRVIGEGWNRPIGRHDPTA
HAEIMALRQGGLVMQNYRLIDATLYVTLEPCVMCAGAMIHSRIGRVVFGARVAKTG
AAGSLMDVLHHPGMNHRVEITEGILADECAALLSDFFRMRRQEIKAQKKAQSSTD
ecTadA (H8Y, D108N, N127S)
(SEQ ID NO: 34)
SEVEFSYEYWMRHALTLAKRAWDEREVPVGAVLVHNNRVIGEGWNRPIGRHDPTA
HAEIMALRQGGLVMQNYRLIDATLYVTLEPCVMCAGAMIHSRIGRVVFGARNAKTG
AAGSLMDVLHHPGMSHRVEITEGILADECAALLSDFFRMRRQEIKAQKKAQSSTD
ecTadA (H8Y, D108N, N127S, E155D)
(SEQ ID NO: 35)
SEVEFSYEYWMRHALTLAKRAWDEREVPVGAVLVHNNRVIGEGWNRPIGRHDPTA
HAEIMALRQGGLVMQNYRLIDATLYVTLEPCVMCAGAMIHSRIGRVVFGARNAKTG
AAGSLMDVLHHPGMSHRVEITEGILADECAALLSDFFRMRRQDIKAQKKAQSSTD
ecTadA (H8Y, D108N, N127S, E155G)
(SEQ ID NO: 36)
SEVEFSYEYWMRHALTLAKRAWDEREVPVGAVLVHNNRVIGEGWNRPIGRHDPTA
HAEIMALRQGGLVMQNYRLIDATLYVTLEPCVMCAGAMIHSRIGRVVFGARNAKTG
AAGSLMDVLHHPGMSHRVEITEGILADECAALLSDFFRMRRQGIKAQKKAQSSTD
ecTadA (H8Y, D108N, N127S, E155V)
(SEQ ID NO: 37)
SEVEFSYEYWMRHALTLAKRAWDEREVPVGAVLVHNNRVIGEGWNRPIGRHDPTA
HAEIMALRQGGLVMQNYRLIDATLYVTLEPCVMCAGAMIHSRIGRVVFGARNAKTG
AAGSLMDVLHHPGMSHRVEITEGILADECAALLSDFFRMRRQVIKAQKKAQSSTD
ecTadA (A106V, D108N, D147Y, and E155V)
(SEQ ID NO: 38)
SEVEFSHEYWMRHALTLAKRAWDEREVPVGAVLVHNNRVIGEGWNRPIGRHDPTA
HAEIMALRQGGLVMQNYRLIDATLYVTLEPCVMCAGAMIHSRIGRVVFGVRNAKTG
AAGSLMDVLHHPGMNHRVEITEGILADECAALLSYFFRMRRQVIKAQKKAQSSTD
ecTadA (S2A, I49F, A106V, D108N, D147Y, E155V)
(SEQ ID NO: 39)
AEVEFSHEYWMRHALTLAKRAWDEREVPVGAVLVHNNRVIGEGWNRPFGRHDPT
AHAEIMALRQGGLVMQNYRLIDATLYVTLEPCVMCAGAMIHSRIGRVVFGVRNAKT
GAAGSLMDVLHHPGMNHRVEITEGILADECAALLSYFFRMRRQVIKAQKKAQSSTD
ecTadA (H8Y, A106T, D108N, N127S, K160S)
(SEQ ID NO: 40)
SEVEFSYEYWMRHALTLAKRAWDEREVPVGAVLVHNNRVIGEGWNRPIGRHDPTA
HAEIMALRQGGLVMQNYRLIDATLYVTLEPCVMCAGAMIHSRIGRVVFGTRNAKTG
AAGSLMDVLHHPGMSHRVEITEGILADECAALLSDFFRMRRQEIKAQSKAQSSTD
ecTadA (R26G, L84F, A106V, R107H, D108N, H123Y, A142N, A143D, D147Y,
E155V, I156F)
(SEQ ID NO: 41)
SEVEFSHEYWMRHALTLAKRAWDEGEVPVGAVLVHNNRVIGEGWNRPIGRHDPTA
HAEIMALRQGGLVMQNYRLIDATLYVTFEPCVMCAGAMIHSRIGRVVFGVHNAKT
GAAGSLMDVLHYPGMNHRVEITEGILADECNDLLSYFFRMRRQVFKAQKKAQSSTD
ecTadA (E25G, R26G, L84F, A106V, R107H, D108N, H123Y, A142N, A143D,
D147Y, E155V, I156F)
(SEQ ID NO: 42)
SEVEFSHEYWMRHALTLAKRAWDGGEVPVGAVLVHNNRVIGEGWNRPIGRHDPTA
HAEIMALRQGGLVMQNYRLIDATLYVTFEPCVMCAGAMIHSRIGRVVFGVHNAKT
GAAGSLMDVLHYPGMNHRVEITEGILADECNDLLSYFFRMRRQVFKAQKKAQSSTD
ecTadA (E25D, R26G, L84F, A106V, R107K, D108N, H123Y, A142N, A143G,
D147Y, E155V, I156F
(SEQ ID NO: 43)
SEVEFSHEYWMRHALTLAKRAWDDGEVPVGAVLVHNNRVIGEGWNRPIGRHDPTA
HAEIMALRQGGLVMQNYRLIDATLYVTFEPCVMCAGAMIHSRIGRVVFGVKNAKT
GAAGSLMDVLHYPGMNHRVEITEGILADECNGLLSYFFRMRRQVFKAQKKAQSSTD
ecTadA (R26Q, L84F, A106V, D108N, H123Y, A142N, D147Y, E155V, I156F
(SEQ ID NO: 44)
SEVEFSHEYWMRHALTLAKRAWDEQEVPVGAVLVHNNRVIGEGWNRPIGRHDPTA
HAEIMALRQGGLVMQNYRLIDATLYVTFEPCVMCAGAMIHSRIGRVVFGVRNAKTG
AAGSLMDVLHYPGMNHRVEITEGILADECNALLSYFFRMRRQVFKAQKKAQSSTD
ecTadA (E25M, R26G, L84F, A106V, R107P, D108N, H123Y, A142N, A143D,
D147Y, E155V, I156F
(SEQ ID NO: 45)
SEVEFSHEYWMRHALTLAKRAWDMGEVPVGAVLVHNNRVIGEGWNRPIGRHDPT
AHAEIMALRQGGLVMQNYRLIDATLYVTFEPCVMCAGAMIHSRIGRVVFGVPNAKT
GAAGSLMDVLHYPGMNHRVEITEGILADECNDLLSYFFRMRRQVFKAQKKAQSSTD
ecTadA (R26C, L84F, A106V, R107H, D108N, H123Y, A142N , D147Y, E155V,
I156F)
(SEQ ID NO: 46)
SEVEFSHEYWMRHALTLAKRAWDECEVPVGAVLVHNNRVIGEGWNRPIGRHDPTA
HAEIMALRQGGLVMQNYRLIDATLYVTFEPCVMCAGAMIHSRIGRVVFGVHNAKT
GAAGSLMDVLHYPGMNHRVEITEGILADECNALLSYFFRMRRQVFKAQKKAQSSTD
ecTadA (L84F, A106V , D108N, H123Y, A142N, A143L, D147Y, E155V, I156F)
(SEQ ID NO: 47)
SEVEFSHEYWMRHALTLAKRAWDEREVPVGAVLVHNNRVIGEGWNRPIGRHDPTA
HAEIMALRQGGLVMQNYRLIDATLYVTFEPCVMCAGAMIHSRIGRVVFGVRNAKTG
AAGSLMDVLHYPGMNHRVEITEGILADECNLLLSYFFRMRRQVFKAQKKAQSSTD
ecTadA (R26G, L84F, A106V, D108N, H123Y, A142N, D147Y, E155V, I156F)
(SEQ ID NO: 48)
SEVEFSHEYWMRHALTLAKRAWDEGEVPVGAVLVHNNRVIGEGWNRPIGRHDPTA
HAEIMALRQGGLVMQNYRLIDATLYVTFEPCVMCAGAMIHSRIGRVVFGVRNAKTG
AAGSLMDVLHYPGMNHRVEITEGILADECNALLSYFFRMRRQVFKAQKKAQSSTD
ecTadA (R51H, L84F, A106V, D108N, H123Y, D147Y, E155V, I156F, K157N)
(SEQ ID NO: 49)
SEVEFSHEYWMRHALTLAKRAWDEREVPVGAVLVHNNRVIGEGWNRPIGHHDPTA
HAEIMALRQGGLVMQNYRLIDATLYVTFEPCVMCAGAMIHSRIGRVVFGVRNAKTG
AAGSLMDVLHYPGMNHRVEITEGILADECAALLSYFFRMRRQVFNAQKKAQSSTD
ecTadA (E25A, R26G, L84F, A106V, R107N, D108N, H123Y, A142N, A143E,
D147Y, E155V, I156F)
(SEQ ID NO: 50)
SEVEFSHEYWMRHALTLAKRAWDAGEVPVGAVLVHNNRVIGEGWNRPIGRHDPTA
HAEIMALRQGGLVMQNYRLIDATLYVTFEPCVMCAGAMIHSRIGRVVFGVNNAKT
GAAGSLMDVLHYPGMNHRVEITEGILADECNELLSYFFRMRRQVFKAQKKAQSSTD
ecTadA (N37T, P48T, L84F, A106V, D108N, H123Y, D147Y, E155V, I156F)
(SEQ ID NO: 51)
SEVEFSHEYWMRHALTLAKRAWDEREVPVGAVLVHTNRVIGEGWNRTIGRHDPTA
HAEIMALRQGGLVMQNYRLIDATLYVTFEPCVMCAGAMIHSRIGRVVFGVRNAKTG
AAGSLMDVLHYPGMNHRVEITEGILADECAALLSYFFRMRRQVFKAQKKAQSSTD
ecTadA (N37S, L84F, A106V, D108N, H123Y, D147Y, E155V, I156F)
(SEQ ID NO: 52)
SEVEFSHEYWMRHALTLAKRAWDEREVPVGAVLVHSNRVIGEGWNRPIGRHDPTA
HAEIMALRQGGLVMQNYRLIDATLYVTFEPCVMCAGAMIHSRIGRVVFGVRNAKTG
AAGSLMDVLHYPGMNHRVEITEGILADECAALLSYFFRMRRQVFKAQKKAQSSTD
ecTadA (H36L, L84F, A106V, D108N, H123Y, D147Y, E155V, I156F)
(SEQ ID NO: 53)
SEVEFSHEYWMRHALTLAKRAWDEREVPVGAVLVLNNRVIGEGWNRPIGRHDPTA
HAEIMALRQGGLVMQNYRLIDATLYVTFEPCVMCAGAMIHSRIGRVVFGVRNAKTG
AAGSLMDVLHYPGMNHRVEITEGILADECAALLSYFFRMRRQVFKAQKKAQSSTD
ecTadA (H36L, P48L, L84F, A106V, D108N, H123Y, D147Y, E155V, I156F)
(SEQ ID NO: 54)
SEVEFSHEYWMRHALTLAKRAWDEREVPVGAVLVLNNRVIGEGWNRLIGRHDPTA
HAEIMALRQGGLVMQNYRLIDATLYVTFEPCVMCAGAMIHSRIGRVVFGVRNAKTG
AAGSLMDVLHYPGMNHRVEITEGILADECAALLSYFFRMRRQVFKAQKKAQSSTD
ecTadA (H36L, L84F, A106V, D108N, H123Y, D147Y, E155V, K57N, I156F)
(SEQ ID NO: 55)
SEVEFSHEYWMRHALTLAKRAWDEREVPVGAVLVLNNRVIGEGWNRPIGRHDPTA
HAEIMALRQGGLVMQNYRLIDATLYVTFEPCVMCAGAMIHSRIGRVVFGVRNAKTG
AAGSLMDVLHYPGMNHRVEITEGILADECAALLSYFFRMRRQVFNAQKKAQSSTD
ecTadA (H36L, L84F, A106V, D108N, H123Y, S146C, D147Y, E155V, I156F)
(SEQ ID NO: 56)
SEVEFSHEYWMRHALTLAKRAWDEREVPVGAVLVLNNRVIGEGWNRPIGRHDPTA
HAEIMALRQGGLVMQNYRLIDATLYVTFEPCVMCAGAMIHSRIGRVVFGVRNAKTG
AAGSLMDVLHYPGMNHRVEITEGILADECAALLCYFFRMRRQVFKAQKKAQSSTD
ecTadA (L84F, A106V, D108N, H123Y, S146R, D147Y, E155V, I156F)
(SEQ ID NO: 57)
SEVEFSHEYWMRHALTLAKRAWDEREVPVGAVLVHNNRVIGEGWNRPIGRHDPTA
HAEIMALRQGGLVMQNYRLIDATLYVTFEPCVMCAGAMIHSRIGRVVFGVRNAKTG
AAGSLMDVLHYPGMNHRVEITEGILADECAALLRYFFRMRRQVFKAQKKAQSSTD
ecTadA (N37S, R51H, L84F, A106V, D108N, H123Y, D147Y, E155V, I156F
(SEQ ID NO: 58)
SEVEFSHEYWMRHALTLAKRAWDEREVPVGAVLVHSNRVIGEGWNRPIGHHDPTA
HAEIMALRQGGLVMQNYRLIDATLYVTFEPCVMCAGAMIHSRIGRVVFGVRNAKTG
AAGSLMDVLHYPGMNHRVEITEGILADECAALLSYFFRMRRQVFKAQKKAQSSTD
ecTadA (R51L, L84F, A106V, D108N, H123Y, D147Y, E155V, 1156F, K157N
(SEQ ID NO: 59)
SEVEFSHEYWMRHALTLAKRAWDEREVPVGAVLVHNNRVIGEGWNRPIGLHDPTA
HAEIMALRQGGLVMQNYRLIDATLYVTFEPCVMCAGAMIHSRIGRVVFGVRNAKTG
AAGSLMDVLHYPGMNHRVEITEGILADECAALLSYFFRMRRQVFNAQKKAQSSTD
ecTadA (P48S)
(SEQ ID NO: 60)
SEVEFSHEYWMRHALTLAKRAWDEREVPVGAVLVHNNRVIGEGWNRSIGRHDPTA
HAEIMALRQGGLVMQNYRLIDATLYVTLEPCVMCAGAMIHSRIGRVVFGARDAKTG
AAGSLMDVLHHPGMNHRVEITEGILADECAALLSDFFRMRRQEIKAQKKAQSSTD
ecTadA (P48T)
(SEQ ID NO: 61)
SEVEFSHEYWMRHALTLAKRAWDEREVPVGAVLVHNNRVIGEGWNRTIGRHDPTA
HAEIMALRQGGLVMQNYRLIDATLYVTLEPCVMCAGAMIHSRIGRVVFGARDAKTG
AAGSLMDVLHHPGMNHRVEITEGILADECAALLSDFFRMRRQEIKAQKKAQSSTD
ecTadA (P48A)
(SEQ ID NO: 62)
SEVEFSHEYWMRHALTLAKRAWDEREVPVGAVLVHNNRVIGEGWNRAIGRHDPTA
HAEIMALRQGGLVMQNYRLIDATLYVTLEPCVMCAGAMIHSRIGRVVFGARDAKTG
AAGSLMDVLHHPGMNHRVEITEGILADECAALLSDFFRMRRQEIKAQKKAQSSTD
ecTadA (A142N)
(SEQ ID NO: 63)
SEVEFSHEYWMRHALTLAKRAWDEREVPVGAVLVHNNRVIGEGWNRPIGRHDPTA
HAEIMALRQGGLVMQNYRLIDATLYVTLEPCVMCAGAMIHSRIGRVVFGARDAKTG
AAGSLMDVLHHPGMNHRVEITEGILADECNALLSDFFRMRRQEIKAQKKAQSSTD
ecTadA (W23R)
(SEQ ID NO: 64)
SEVEFSHEYWMRHALTLAKRARDEREVPVGAVLVHNNRVIGEGWNRPIGRHDPTA
HAEIMALRQGGLVMQNYRLIDATLYVTLEPCVMCAGAMIHSRIGRVVFGARDAKTG
AAGSLMDVLHHPGMNHRVEITEGILADECAALLSDFFRMRRQEIKAQKKAQSSTD
ecTadA (W23L)
(SEQ ID NO: 65)
SEVEFSHEYWMRHALTLAKRALDEREVPVGAVLVHNNRVIGEGWNRPIGRHDPTA
HAEIMALRQGGLVMQNYRLIDATLYVTLEPCVMCAGAMIHSRIGRVVFGARDAKTG
AAGSLMDVLHHPGMNHRVEITEGILADECAALLSDFFRMRRQEIKAQKKAQSSTD
ecTadA (R152P)
(SEQ ID NO: 66)
SEVEFSHEYWMRHALTLAKRAWDEREVPVGAVLVHNNRVIGEGWNRPIGRHDPTA
HAEIMALRQGGLVMQNYRLIDATLYVTLEPCVMCAGAMIHSRIGRVVFGARDAKTG
AAGSLMDVLHHPGMNHRVEITEGILADECAALLSDFFRMPRQEIKAQKKAQSSTD
ecTadA (R152H)
(SEQ ID NO: 67)
SEVEFSHEYWMRHALTLAKRAWDEREVPVGAVLVHNNRVIGEGWNRPIGRHDPTA
HAEIMALRQGGLVMQNYRLIDATLYVTLEPCVMCAGAMIHSRIGRVVFGARDAKTG
AAGSLMDVLHHPGMNHRVEITEGILADECAALLSDFFRMHRQEIKAQKKAQSSTD
ecTadA (L84F, A106V, D108N, H123Y, D147Y, E155V, I156F)
(SEQ ID NO: 68)
SEVEFSHEYWMRHALTLAKRAWDEREVPVGAVLVHNNRVIGEGWNRPIGRHDPTA
HAEIMALRQGGLVMQNYRLIDATLYVTFEPCVMCAGAMIHSRIGRVVFGVRNAKTG
AAGSLMDVLHYPGMNHRVEITEGILADECAALLSYFFRMRRQVFKAQKKAQSSTD
ecTadA (H36L, R51L, L84F, A106V, D108N, H123Y, S146C, D147Y, E155V,
I156F, K157N)
(SEQ ID NO: 69)
SEVEFSHEYWMRHALTLAKRAWDEREVPVGAVLVLNNRVIGEGWNRPIGLHDPTA
HAEIMALRQGGLVMQNYRLIDATLYVTFEPCVMCAGAMIHSRIGRVVFGVRNAKTG
AAGSLMDVLHYPGMNHRVEITEGILADECAALLCYFFRMRRQVFNAQKKAQSSTD
ecTadA (H36L, P48S, R51L, L84F, A106V, D108N, H123Y, S146C, D147Y,
E155V, I156F, K157N)
(SEQ ID NO: 70)
SEVEFSHEYWMRHALTLAKRAWDEREVPVGAVLVLNNRVIGEGWNRSIGLHDPTA
HAEIMALRQGGLVMQNYRLIDATLYVTFEPCVMCAGAMIHSRIGRVVFGVRNAKTG
AAGSLMDVLHYPGMNHRVEITEGILADECAALLCYFFRMRRQVFNAQKKAQSSTD
ecTadA (H36L, P48A, R51L, L84F, A106V, D108N, H123Y, S146C, D147Y,
E155V, I156F, K157N)
(SEQ ID NO: 71)
SEVEFSHEYWMRHALTLAKRAWDEREVPVGAVLVLNNRVIGEGWNRAIGLHDPTA
HAEIMALRQGGLVMQNYRLIDATLYVTFEPCVMCAGAMIHSRIGRVVFGVRNAKTG
AAGSLMDVLHYPGMNHRVEITEGILADECAALLCYFFRMRRQVFNAQKKAQSSTD
ecTadA (W23L, H36L, P48A, R51L, L84F, A106V, D108N, H123Y, S146C,
D147Y, R152P, E155V, I156F, K157N)
(SEQ ID NO: 72)
SEVEFSHEYWMRHALTLAKRALDEREVPVGAVLVLNNRVIGEGWNRAIGLHDPTA
HAEIMALRQGGLVMQNYRLIDATLYVTFEPCVMCAGAMIHSRIGRVVFGVRNAKTG
AAGSLMDVLHYPGMNHRVEITEGILADECAALLCYFFRMPRQVFNAQKKAQSSTD
ecTadA (W23R, H36L, P48A, R51L, L84F, A106V, D108N, H123Y, S146C,
D147Y, R152P, E155V, I156F, K157N) (also known as TadA 7.10)
(SEQ ID NO: 73)
SEVEFSHEYWMRHALTLAKRARDEREVPVGAVLVLNNRVIGEGWNRAIGLHDPTA
HAEIMALRQGGLVMQNYRLIDATLYVTFEPCVMCAGAMIHSRIGRVVFGVRNAKTG
AAGSLMDVLHYPGMNHRVEITEGILADECAALLCYFFRMPRQVFNAQKKAQSSTD
TadA 7.10 (V106W) (E. coli)
(SEQ ID NO: 74)
SEVEFSHEYWMRHALTLAKRARDEREVPVGAVLVLNNRVIGEGWNRAIGLHDPTA
HAEIMALRQGGLVMQNYRLIDATLYVTFEPCVMCAGAMIHSRIGRVVFGWRNAKT
GAAGSLMDVLHYPGMNHRVEITEGILADECAALLCYFFRMPRQVFNAQKKAQSSTD
TadA-8e (E. coli)
(SEQ ID NO: 75)
SEVEFSHEYWMRHALTLAKRARDEREVPVGAVLVLNNRVIGEGWNRAIGLHDPTA
HAEIMALRQGGLVMQNYRLIDATLYVTFEPCVMCAGAMIHSRIGRVVFGVRNSKRG
AAGSLMNVLNYPGMNHRVEITEGILADECAALLCDFYRMPRQVFNAQKKAQSSIN
TadA-8e(V106W) (E. coli)
(SEQ ID NO: 76)
SEVEFSHEYWMRHALTLAKRARDEREVPVGAVLVLNNRVIGEGWNRAIGLHDPTA
HAEIMALRQGGLVMQNYRLIDATLYVTFEPCVMCAGAMIHSRIGRVVFGWRNSKR
GAAGSLMNVLNYPGMNHRVEITEGILADECAALLCDFYRMPRQVFNAQKKAQSSIN

In some embodiments, the base editor is an adenosine base editor. In some embodiments, a base editor comprises at least two adenosine deaminase domains. Without wishing to be bound by any particular theory, dimerization of adenosine deaminases (e.g., in cis or in trans) may improve the ability (e.g., efficiency) of the base editor to modify a nucleic acid base (for example, to deaminate adenosine). In some embodiments, any of the base editors provided herein comprise 2, 3, 4, or 5 adenosine deaminase domains. In some embodiments, any of the base editors provided herein comprise two adenosine deaminases. In certain embodiments, the adenosine deaminases are the same. In some embodiments, the adenosine deaminases are any of the adenosine deaminases provided herein. In certain embodiments, the adenosine deaminases are different. Other adenosine deaminase domains besides those provided herein are known in the art, and a person of ordinary skill in the art would recognize which adenosine deaminase domains could be used in the fusion proteins of the present disclosure.

In some embodiments, the general architecture of the base editors contemplated by the present disclosure comprises any one of the following structures: NH₂-[first adenosine deaminase]-[second adenosine deaminase]-[napDNAbp]-COOH; NH₂-[first adenosine deaminase]-[napDNAbp]-[second adenosine deaminase]-COOH; NH₂-[napDNAbp]-[first adenosine deaminase]-[second adenosine deaminase]-COOH; NH₂-[second adenosine deaminase]-[first adenosine deaminase]-[napDNAbp]-COOH; NH₂-[second adenosine deaminase]-[napDNAbp]-[first adenosine deaminase]-COOH; NH₂-[napDNAbp]-[second adenosine deaminase]-[first adenosine deaminase]-COOH. In certain embodiments, the general architecture of the base editor comprises the structure NH₂-[first adenosine deaminase]-[second adenosine deaminase]-[napDNAbp]-COOH.

In various embodiments, the base editors used in the present disclosure may be fused to one or more nuclear localization sequences (NLS), which help promote translocation of the base editor into the cell nucleus. In some embodiments, the base editors described herein may comprise one or more NLS. Such sequences are well-known in the art and can include the following examples:

| NLS Sequence | SEQ ID NO: |
|---|---|
| PKKKRKV | 77 |
| MKRTADGSEFESPKKKRKV | 78 |
| MDSLLMNRRKFLYQFKNVRWAKGRRETYLC | 79 |
| AVKRPAATKKAGQAKKKKLD | 80 |
| MSRRRKANPTKLSENAKKLAKEVEN | 81 |
| PAAKRVKLD | 82 |
| KLKIKRPVK | 83 |
| VSRKRPRP | 84 |
| EGAPPAKRAR | 85 |
| PPQPKKKPLDGE | 86 |
| KRTADGSEFEPKKKRKV | 87 |
| KRTADGSEFESPKKKRKV | 88 |

The NLS examples above are non-limiting. The fusion proteins provided herein may comprise any known NLS sequence, including any of those described in Cokol et al., “Finding nuclear localization signals,” EMBO Rep., 2000, 1(5): 411-415; and Freitas et al., “Mechanisms and Signals for the Nuclear Import of Proteins,” Current Genomics, 2009, 10(8): 550-7, each of which are incorporated herein by reference.

In various embodiments, the base editors and constructs encoding the base editors disclosed herein further comprise one or more, preferably at least two, nuclear localization sequences. In certain embodiments, the base editors comprise at least two NLSs. In embodiments with at least two NLSs, the NLSs can be the same NLSs, or they can be different NLSs. In some embodiments, one or more of the NLSs are bipartite NLSs (“bpNLS”). In certain embodiments, the disclosed base editors comprise two bipartite NLSs. In some embodiments, the disclosed base editors comprise more than two bipartite NLSs. The location of the NLS fusion can be at the N-terminus, the C-terminus, or within a sequence of a base editor

In certain embodiments, a base editor comprises an NLS of the amino acid sequence PKKKRKV (SEQ ID NO: 77). In certain embodiments, a base editor comprises an NLS of the amino acid sequence MKRTADGSEFESPKKKRKV (SEQ ID NO: 78). In certain embodiments, a base editor comprises an NLS of the amino acid sequence KRTADGSEFEPKKKRKV (SEQ ID NO: 87).

Exemplary base editor fusion architectures comprising a first adenosine deaminase, a second adenosine deaminase, a napDNAbp, and an NLS are provided: NH₂-[NLS]-[first adenosine deaminase]-[second adenosine deaminase]-[napDNAbp]-COOH; NH₂-[first adenosine deaminase]-[NLS]-[second adenosine deaminase]-[napDNAbp]-COOH; NH₂-[first adenosine deaminase]-[second adenosine deaminase]-[NLS]-[napDNAbp]-COOH; NH₂-[first adenosine deaminase]-[second adenosine deaminase]-[napDNAbp]-[NLS]-COOH; NH₂-[NLS]-[first adenosine deaminase]-[napDNAbp]-[second adenosine deaminase]-COOH; NH₂-[first adenosine deaminase]-[NLS]-[napDNAbp]-[second adenosine deaminase]-COOH; NH₂-[first adenosine deaminase]-[napDNAbp]-[NLS]-[second adenosine deaminase]-COOH; NH₂-[first adenosine deaminase]-[napDNAbp]-[second adenosine deaminase]-[NLS]-COOH; NH₂-[NLS]-[napDNAbp]-[first adenosine deaminase]-[second adenosine deaminase]-COOH; NH₂-[napDNAbp]-[NLS]-[first adenosine deaminase]-[second adenosine deaminase]-COOH; NH₂-[napDNAbp]-[first adenosine deaminase]-[NLS]-[second adenosine deaminase]-COOH; NH₂-[napDNAbp]-[first adenosine deaminase]-[second adenosine deaminase]-[NLS]-COOH; NH₂-[NLS]-[second adenosine deaminase]-[first adenosine deaminase]-[napDNAbp]-COOH; NH₂-[second adenosine deaminase]-[NLS]-[first adenosine deaminase]-[napDNAbp]-COOH; NH₂-[second adenosine deaminase]-[first adenosine deaminase]-[NLS]-[napDNAbp]-COOH; NH₂-[second adenosine deaminase]-[first adenosine deaminase]-[napDNAbp]-[NLS]-COOH; NH₂-[NLS]-[second adenosine deaminase]-[napDNAbp]-[first adenosine deaminase]-COOH; NH₂-[second adenosine deaminase]-[NLS]-[napDNAbp]-[first adenosine deaminase]-COOH; NH₂-[second adenosine deaminase]-[napDNAbp]-[NLS]-[first adenosine deaminase]-COOH; NH₂-[second adenosine deaminase]-[napDNAbp]-[first adenosine deaminase]-[NLS]-COOH; NH₂-[NLS]-[napDNAbp]-[second adenosine deaminase]-[first adenosine deaminase]-COOH; NH₂-[napDNAbp]-[NLS]-[second adenosine deaminase]-[first adenosine deaminase]-COOH; NH₂-[napDNAbp]-[second adenosine deaminase]-[NLS]-[first adenosine deaminase]-COOH; NH₂-[napDNAbp]-[second adenosine deaminase]-[first adenosine deaminase]-[NLS]-COOH.

In some embodiments, each instance of “]-[” used in the general architecture above indicates the presence of an optional linker. In some embodiments, a base editor comprises one or more a peptide linkers. Exemplary peptide linkers for use in the base editors contemplated by the present disclosure include, but are not limited to, (GGGGS)ₙ (SEQ ID NO: 89), (G)ₙ (SEQ ID NO: 90), (EAAAK)ₙ (SEQ ID NO: 91), (GGS)ₙ (SEQ ID NO: 92), (SGGS)ₙ (SEQ ID NO: 93), (XP)ₙ (SEQ ID NO: 94), SGSETPGTSESATPES (SEQ ID NO: 95), SGSETPGTSESA (SEQ ID NO: 96), SGSETPGTSESATPEGGSGGS (SEQ ID NO: 97), SGGSSGGSSGSETPGTSESATPESSGGSSGGS (SEQ ID NO: 98), SGGSGGSGGS (SEQ ID NO: 99), SGGS (SEQ ID NO: 100), SGGSSGGSSGSETPGTSESATPESAGSYPYDVPDYAGSAAPAAKKKKLDGSGSGGSS GGS (SEQ ID NO: 101), GGSGGS (SEQ ID NO: 102), GGSGGSGGS (SEQ ID NO: 103), SGGSSGGSSGSETPGTSESATPESSGGSSGGSS (SEQ ID NO: 104), SGGSSGGSSGSETPGTSESATPESAGSYPYDVPDYAGSAAPAAKKKKLDGSGSGGSS GG S (SEQ ID NO: 101), or any combination thereof, wherein n is independently an integer between 1 and 30, and wherein X is any amino acid.

In certain embodiments, a base editor useful in the present disclosure is ABE7.10 (SEQ ID NO: 105), or comprises an amino acid sequence at least 80%, at least 85%, at least 90%, at least 95%, at least 96%, at least 97%, at least 98%, or at least 99% identical to the amino acid sequence of ABE7.10 (SEQ ID NO: 105):

(SEQ ID NO: 105)
MSEVEFSHEYWMRHALTLAKRAWDEREVPVGAVLVHNNRVIGEGWNRPIGRHDPT
AHAEIMALRQGGLVMQNYRLIDATLYVTLEPCVMCAGAMIHSRIGRVVFGARDAKT
GAAGSLMDVLHHPGMNHRVEITEGILADECAALLSDFFRMRRQEIKAQKKAQSSTD
SGGSSGGSSGSETPGTSESATPESSGGSSGGSSEVEFSHEYWMRHALTLAKRARDERE
VPVGAVLVLNNRVIGEGWNRAIGLHDPTAHAEIMALRQGGLVMQNYRLIDATLYVT
FEPCVMCAGAMIHSRIGRVVFGVRNAKTGAAGSLMDVLHYPGMNHRVEITEGILAD
ECAALLCYFFRMPRQVFNAQKKAQSSTDSGGSSGGSSGSETPGTSESATPESSGGSSG
GSDKKYSIGLAIGTNSVGWAVITDEYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECF
DSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDEL
VKVMGRHKPENIVIEMARENQTTQKGQKNSRERMKRIEEGIKELGSQILKEHPVENT
QLQNEKLYLYYLQNGRDMYVDQELDINRLSDYDVDHIVPQSFLKDDSIDNKVLTRS
DKNRGKSDNVPSEEVVKKMKNYWRQLLNAKLITQRKFDNLTKAERGGLSELDKAG
FIKRQLVETR QITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLF
VEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGDSGGSP
KKKRKV

In certain embodiments, a base editor useful in the present disclosure is ABE8e (SEQ ID NO: 106), or comprises an amino acid sequence at least 80%, at least 85%, at least 90%, at least 95%, at least 96%, at least 97%, at least 98%, or at least 99% identical to the amino acid sequence of ABE8e (SEQ ID NO: 106):

(SEQ ID NO: 106)
MKRTADGSEFESPKKKRKVSEVEFSHEYWMRHALTLAKRARDEREVPVGAVLVLN
NRVIGEGWNRAIGLHDPTAHAEIMALRQGGLVMQNYRLIDATLYVTFEPCVMCAGA
MIHSRIGRVVFGVRNSKRGAAGSLMNVLNYPGMNHRVEITEGILADECAALLCDFY
RMPRQVFNAQKKAQSSINSGGSSGGSSGSETPGTSESATPESSGGSSGGSDKKYSIGL
AIGTNSVGWAVITDEYKVPSKKFKVLGNTDRHSIKKNLIGALLFDSGETAEATRLKR
TARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEERLKTYAHLF
DDKVMKQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNV
PSEEVVKKMKNYWRQLLNAKLITQRKFDNLTKAERGGLSELDKAGFIKRQLVETRQ
ITKHVAQILDSRMNTKYDENDKLIREVKVITLKSKLVSDFRKDFQFYKVREINNYHH
AHDAYLNAVVGTALIKKYPKLESEFVYGDYKVYDVRKMIAKSEQEIGKATAKYFFY
SNIMNFFKTEITLANGEIRKRPLIETNGETGEIVWDKGRDFATVRKVLSMPQVNIVKK
TEVQTGGFSKESILPKRNSDKLIARKKDWDPKKYGGFDSPTVAYSVLVVAKVEKGK
SKKLKSVKELLGITIMERSSFEKNPIDFLEAKGYKEVKKDLIIKLPKYSLFELENGRKR
MLASAGELQKGNELALPSKYVNFLYLASHYEKLKGSPEDNEQKQLFVEQHKHYLDE
IIEQISEFSKRVILADANLDKVLSAYNKHRDKPIREQAENIIHLFTLTNLGAPAAFKYF
DTTIDRKRYTSTKEVLDATLIHQSITGLYETRIDLSQLGGDSGGSKRTADGSEFEPKK
KRKV

In certain embodiments, a base editor useful in the present disclosure is ABE8e(V106W) (SEQ ID NO: 107), or comprises an amino acid sequence at least 80%, at least 85%, at least 90%, at least 95%, at least 96%, at least 97%, at least 98%, or at least 99% identical to the amino acid sequence of ABE8e(V106W) (SEQ ID NO: 107):

(SEQ ID NO: 107)
MKRTADGSEFESPKKKRKVSEVEFSHEYWMRHALTLAKRARDEREVPVGAVLVLN
NRVIGEGWNRAIGLHDPTAHAEIMALRQGGLVMQNYRLIDATLYVTFEPCVMCAGA
MIHSRIGRVVFGWRNSKRGAAGSLMNVLNYPGMNHRVEITEGILADECAALLCDFY
RMPRQVFNAQKKAQSSINSGGSSGGSSGSETPGTSESATPESSGGSSGGSDKKYSIGL
AIGTNSVGWAVITDEYKVPSKKFKVLGNTDRHSIKKNLIGALLFDSGETAEATRLKR
TARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQS
KNGYAGYIDGGASQEEFYKFIKPILEKMDGTEELLVKLNREDLLRKQRTFDNGSIPH
QIHLGELHAILRRQEDFYPFLKDNREKIEKILTFRIPYYVGPLARGNSRFAWMTRKSE
ETITPWNFEEVVDKGASAQSFIERMTNFDKNLPNEKVLPKHSLLYEYFTVYNELTKV
KYVTEGMRKPAFLSGEQKKAIVDLLFKTNRKVTVKQLKEDYFKKIECFDSVEISGVE
DRFNASLGTYHDLLKIIKDKDFLDNEENEDILEDIVLTLTLFEDREMIEERLKTYAHLF
DDKVMKQLKRRRYTGWGRLSRKLINGIRDKQSGKTILDFLKSDGFANRNFMQLIHD
DSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNV
PSEEVVKKMKNYWRQLLNAKLITQRKFDNLTKAERGGLSELDKAGFIKRQLVETRQ
ITKHVAQILDSRMNTKYDENDKLIREVKVITLKSKLVSDFRKDFQFYKVREINNYHH
AHDAYLNAVVGTALIKKYPKLESEFVYGDYKVYDVRKMIAKSEQEIGKATAKYFFY
SNIMNFFKTEITLANGEIRKRPLIETNGETGEIVWDKGRDFATVRKVLSMPQVNIVKK
TEVQTGGFSKESILPKRNSDKLIARKKDWDPKKYGGFDSPTVAYSVLVVAKVEKGK
SKKLKSVKELLGITIMERSSFEKNPIDFLEAKGYKEVKKDLIIKLPKYSLFELENGRKR
MLASAGELQKGNELALPSKYVNFLYLASHYEKLKGSPEDNEQKQLFVEQHKHYLDE
IIEQISEFSKRVILADANLDKVLSAYNKHRDKPIREQAENIIHLFTLTNLGAPAAFKYF
DTTIDRKRYTSTKEVLDATLIHQSITGLYETRIDLSQLGGDSGGSKRTADGSEFEPKK
KRKV

### Methods of Base Editing Tpp1

Some aspects of the present disclosure provide methods of base editing a Tpp1 gene. In one aspect, the present disclosure provides methods of base editing a Tpp1 gene comprising contacting a nucleic acid sequence encoding the Tpp1 gene with a base editor and a guide RNA (gRNA) targeting the base editor to the Tpp1 gene. In some embodiments, the gRNA targets a strand complementary to a protospacer in a Tpp1 gene comprising a nucleotide sequence at least 80%, at least 85%, at least 90%, at least 95%, at least 96%, at least 97%, at least 98%, or at least 99% identical to the sequence TATCACTTACGGATCACAGA (SEQ ID NO: 2), or a fragment thereof. In some embodiments, the gRNA targets a sequence in a Tpp1 gene that is shifted upstream or downstream relative to SEQ ID NO: 2 in the human Tpp1 gene of SEQ ID NO: 8 (e.g., shifted upstream or downstream by 1, 2, 3, 4, 5, 6, 7, 8, 9, or 10 nucleotides). In certain embodiments, the gRNA targets a strand complementary to a protospacer in the Tpp1 gene comprising the nucleotide sequence TATCACTTACGGATCACAGA (SEQ ID NO: 2). In some embodiments, the gRNA comprises a spacer targeting the gRNA to a human Tpp1 gene. In some embodiments, the gRNA comprises a spacer at least 80%, at least 85%, at least 90%, at least 95%, at least 96%, at least 97%, at least 98%, or at least 99% identical to the sequence GTATCACTTACGGATCACAGA (SEQ ID NO: 4), or a fragment thereof. In certain embodiments, the gRNA comprises a spacer of the sequence GTATCACTTACGGATCACAGA (SEQ ID NO: 4).

In some embodiments, the gRNA targets a strand complementary to a protospacer in a Tpp1 gene comprising a nucleotide sequence at least 80%, at least 85%, at least 90%, at least 95%, at least 96%, at least 97%, at least 98%, or at least 99% identical to the sequence TATCACTGACGGAGCACAGA (SEQ ID NO: 1), or a fragment thereof. In some embodiments, the gRNA targets a sequence in a Tpp1 gene that is shifted upstream or downstream relative to SEQ ID NO: 1 in the mouse Tpp1 gene of SEQ ID NO: 10 (e.g., shifted upstream or downstream by 1, 2, 3, 4, 5, 6, 7, 8, 9, or 10 nucleotides). In certain embodiments, the gRNA targets a strand complementary to a protospacer in the Tpp1 gene comprising the nucleotide sequence TATCACTGACGGAGCACAGA (SEQ ID NO: 1). In some embodiments, the gRNA comprises a spacer targeting the gRNA to a mouse Tpp1 gene. In some embodiments, the gRNA comprises a spacer at least 80%, at least 85%, at least 90%, at least 95%, at least 96%, at least 97%, at least 98%, or at least 99% identical to the sequence GTATCACTGACGGAGCACAGA (SEQ ID NO: 3), or a fragment thereof. In certain embodiments, the gRNA comprises a spacer of the sequence GTATCACTGACGGAGCACAGA (SEQ ID NO: 3).

In some embodiments, the gRNA comprises a backbone scaffold at least 80%, at least 85%, at least 90%, at least 95%, at least 96%, at least 97%, at least 98%, or at least 99% identical to the sequence GTTTTAGAGCTAGAAATAGCAAGTTAAAATAAGGCTAGTCCGTTATCAACTTGAA AAAGTGGCACCGAGTCGGTGC (SEQ ID NO: 5), or a fragment thereof. In certain embodiments, the gRNA comprises a backbone scaffold of the sequence GTTTTAGAGCTAGAAATAGCAAGTTAAAATAAGGCTAGTCCGTTATCAACTTGAA AAAGTGGCACCGAGTCGGTGC (SEQ ID NO: 5), or a fragment thereof.

In some embodiments, the gRNA comprises a sequence at least 80%, at least 85%, at least 90%, at least 95%, at least 96%, at least 97%, at least 98%, or at least 99% identical to the sequence GTATCACTTACGGATCACAGAGTTTTAGAGCTAGAAATAGCAAGTTAAAATAAG GCTAGTCCGTTATCAACTTGAAAAAGTGGCACCGAGTCGGTGC (SEQ ID NO: 6) or GTATCACTGACGGAGCACAGAGTTTTAGAGCTAGAAATAGCAAGTTAAAATAAG GCTAGTCCGTTATCAACTTGAAAAAGTGGCACCGAGTCGGTGC (SEQ ID NO: 7). In certain embodiments, the gRNA comprises the sequence

(SEQ ID NO: 6)
GTATCACTTACGGATCACAGAGTTTTAGAGCTAGAAATAGCAAGTTAAAA
TAAGGCTAGTCCGTTATCAACTTGAAAAAGTGGCACCGAGTCGGTGC
or
(SEQ ID NO: 7)
GTATCACTGACGGAGCACAGAGTTTTAGAGCTAGAAATAGCAAGTTAAAA
TAAGGCTAGTCCGTTATCAACTTGAAAAAGTGGCACCGAGTCGGTGC.

Any of the base editors disclosed herein, or any base editor known in the art, can be used in the methods of the present disclosure. In some embodiments, the base editor is an adenosine base editor. In some embodiments, the base editor comprises a napDNAbp (e.g., a Cas9 protein, such as SpCas9, or a variant thereof, such as nCas9 or dCas9) and a deaminase (e.g., an adenosine deaminase, such as an ecTadA deaminase, or a variant thereof). In certain embodiments, the base editor is ABE7.10, ABE8e, ABE8e(V106W), or a variant thereof. In certain embodiments, the base editor is ABE7.10.

In some embodiments, the nucleic acid sequence encoding the Tpp1 gene comprises at least one mutation associated with a disease or disorder (e.g., Batten disease, including CLN2). In some embodiments, the Tpp1 gene comprises a point mutation associated with a disease or disorder (e.g., Batten disease). In some embodiments, the Tpp1 gene comprises a G→A point mutation associated with a disease or disorder, and the deamination of the mutant A base results in a sequence that is not associated with a disease or disorder. In certain embodiments, the mutation is a C·G-to-T·A transition mutation. In some embodiments, the methods provided herein result in correction of a C·G-to-T·A transition mutation in a Tpp1 gene. In certain embodiments, correction of the C·G-to-T·A transition mutation in a human Tpp1 gene results in correction of an R208X mutation in a human Tpp1 protein of SEQ ID NO: 9, where X is a premature stop codon. In certain embodiments, correction of the C·G-to-T·A transition mutation in a mouse Tpp1 gene results in correction of an R207X mutation in a mouse Tpp1 protein of SEQ ID NO: 11, where X is a premature stop codon.

In some embodiments, the contacting step comprises delivering one or more polynucleotides encoding the gRNA and the base editor to the nucleic acid sequence encoding the Tpp1 gene (e.g., in one or more AAV particles as described further herein). In some embodiments, the contacting step is performed in a cell, such as a human or non-human animal cell. In some embodiments, the contacting step is performed in vitro. In some embodiments, the contacting step is performed in vivo. In certain embodiments, the contacting step is performed in a subject. In some embodiments the contacting is performed in a cell in the central nervous system (CNS) of the subject. In some embodiments, the contacting is performed in neurons in a subject. A subject may have been diagnosed with a disease, or be at risk for having a disease. In some embodiments, the method is a method for treating a disease in a subject. In some embodiments, the disease is a lysosomal storage disease. In some embodiments, the disease is a neuronal ceroid lipofuscinosis. In certain embodiments, the disease is late infantile neuronal ceroid lipofuscinosis type 2 (CLN2). In some embodiments, the disease is Batten disease. In some embodiments, the method is a method of treating Tpp1 R208X-mediated Batten disease. In certain embodiments, the method prevents or reduces the severity of neural degeneration, ataxia, epilepsy, and/or blindness in the subject. In certain embodiments, the method results in increased Tpp1 enzyme activity in the subject. In some embodiments, the subject is a human. In some embodiments, the subject is an infant. In some embodiments, the subject is less than ten, less than nine, less than eight, less than seven, less than sex, less than five, less than four, less than three, or less than two years old. In certain embodiments, the subject is less than four years old. In certain embodiments, the subject is between two and four years old.

In some aspects, the present disclosure contemplates use of any of the gRNAs, complexes, AAV particles, polynucleotides, vectors, pharmaceutical compositions, and/or cells disclosed herein in the manufacture of a medicament for the treatment of a disease or disorder (e.g., Batten disease). In some aspects, any of the gRNAs, complexes, AAV particles, polynucleotides, vectors, pharmaceutical compositions, and/or cells disclosed herein are for use in medicine. In some embodiments, the present disclosure provides for veterinary uses (e.g., in non-human animals) of any of the gRNAs, complexes, AAV particles, polynucleotides, vectors, pharmaceutical compositions, cells, and/or methods provided herein.

### Delivery Methods and AAV Particles

The present disclosure provides, in some aspects, methods comprising delivering any of the gRNAs, complexes, polynucleotides, vectors, and pharmaceutical compositions described herein. In some embodiments, a gRNA is delivered to a cell, e.g., in combination with a base editor. The base editor and/or gRNA can be delivered in any form, e.g., each may independently be delivered in DNA, RNA, or (for the base editor) protein form. Conventional viral and non-viral based gene transfer methods can be used to introduce nucleic acids in cells (e.g., mammalian cells) or target tissues. Such methods can be used to administer nucleic acids encoding components of a base editor and gRNA to cells in culture, or in a host organism. Non-viral vector delivery systems include ribonucleoprotein (RNP) complexes, DNA plasmids, RNA, naked nucleic acid, and nucleic acid complexed with, part of, or associated with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bihm (eds) (1995); and Yu et al., Gene Therapy 1:13-26 (1994).

In some embodiments, the gRNA and base editor are delivered or administered as a protein:RNA complex. In certain embodiments, the method of delivery comprises delivering an RNP complex. For example, RNP delivery of base editors markedly increases the DNA specificity of base editing. RNP delivery of base editors leads to fewer off-target effects. RNP delivery ablated off-target editing at non-repetitive sites while maintaining on-target editing comparable to plasmid delivery, and greatly reduced off-target editing even at the highly repetitive VEGFA site 2. See Rees, H. A. et al., Improving the DNA specificity and applicability of base editing through protein engineering and protein delivery, Nat. Commun. 8, 15790 (2017), which is incorporated herein by reference.

Methods of non-viral delivery of nucleic acids include RNP complexes, lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in, e.g., U.S. Pat. Nos. 5,049,386, 4,946,787, and 4,897,355, and lipofection reagents are sold commercially (e.g., Lipofectamine, Lipofectamine 2000, Lipofectamine 3000, TransfectamT^{M} and Lipofectin™) In certain embodiments of the disclosed methods of editing, a cationic lipid comprising Lipofectamine 2000 is used for delivery of nucleic acids to cells. Cationic and neutral lipids that are suitable for efficient receptor-recognition lipofection of polynucleotides include those of Feigner (see WO 1991/17424 and WO 1991/16024). Delivery of, e.g., Cas9 proteins and gRNAs using cationic lipids and cationic polymers is also described in International Patent Application Publication Nos. WO 2015/035136 and WO 2016/070129, each of which is incorporate herein by reference. Delivery can be to cells (e.g., in vitro or ex vivo administration) or target tissues (e.g., in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, 4,946,787, 9,526,784, and 9,737,604).

The use of RNA or DNA viral based systems for the delivery of nucleic acids (e.g., nucleic acids encoding a base editor and gRNA as described herein) take advantage of highly evolved processes for targeting a virus to specific cells in the body and trafficking the viral payload to the nucleus. Viral vectors can be administered directly to patients (in vivo), or they can be used to treat cells in vitro, and the modified cells may optionally be administered to patients (ex vivo). Conventional viral based systems could include retroviral, lentivirus, adenoviral, adeno-associated, and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

In some embodiments, an adeno-associated virus (AAV)-based system is used for delivery of nucleic acid molecule(s) encoding a gRNA and base editor. Particularly in applications where transient expression is preferred, adenoviral-based systems may be used. Adenoviral-based vectors are capable of very high transduction efficiency in many different cell types and do not require cell division. With such vectors, high titer and levels of expression have been obtained. This vector can be produced in large quantities in a relatively simple system. AAV vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); Samulski et al., J. Virol. 63:03822-3828 (1989); and International Patent Application No. PCT/US2023/066389, filed Apr. 28, 2023.

Packaging cells are typically used to form virus particles that are capable of infecting a host cell. Such cells include 293 cells, which package adenovirus, and W² cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. In some embodiments, the AAV targets the central nervous system (CNS). In some embodiments, the AAV targets neurons. In certain embodiments, the AAV is AAV9.

In various embodiments, the constructs for expressing a gRNA and base editor described herein may be engineered for delivery in one or more AAV vectors. An AAV as related to any of the methods and compositions provided herein may be of any serotype including any derivative or pseudotype (e.g., 1, 2, 3, 4, 5, 6, 7, 8, 9, 10, 11, 12, 13, 2/1, 2/5, 2/8, 2/9, 3/1, 3/5, 3/8, or 3/9). An AAV may comprise a genetic load (i.e., a recombinant nucleic acid vector that expresses gene products of interest, such as a base editor and/or gRNA that is carried by the AAV into a cell) that is to be delivered to a cell.

In one aspect, the present disclosure provides one or more AAV particles comprising one or more polynucleotides encoding any of the gRNAs and base editors, or portion(s) thereof, provided herein. In some embodiments, the polynucleotide encoding the base editor is split between a first and a second AAV particle. In certain embodiments, the polynucleotides encoding the split base editor comprise an N-intein and a C-intein. In some embodiments, the first and/or the second AAV particle further comprises the polynucleotide encoding the gRNA.

In some embodiments, a first AAV particle contains a polynucleotide comprising a sequence at least 80%, at least 85%, at least 90%, at least 95%, at least 96%, at least 97%, at least 98%, or at least 99% identical to SEQ ID NO: 108 or 109: AAV vector sequence comprising ABE7.10-SpCas9 amino acids 1-572 N-intein:

(SEQ ID NO: 108)
CTGCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGGGA
GTGGCCAACTCCATCACTAGGGGTTCCTGCGGCCTCTAGATCAGGGTACCC
GTTACATAACTTACGGTAAATGGCCCGCCTGGCTGACCGCCCAACGACCCC
CGCCCATTGACGTCAATAGTAACGCCAATAGGGACTTTCCATTGACGTCAA
TGGGTGGAGTATTTACGGTAAACTGCCCACTTGGCAGTACATCAAGTGTAT
CATATGCCAAGTACGCCCCCTATTGACGTCAATGACGGTAAATGGCCCGCC
TGGCATTGTGCCCAGTACATGACCTTATGGGACTTTCCTACTTGGCAGTAC
ATCTACGTATTAGTCATCGCTATTACCATGGTCGAGGTGAGCCCCACGTTC
TGCTTCACTCTCCCCATCTCCCCCCCCTCCCCACCCCCAATTTTGTATTTA
TTTATTTTTTAATTATTTTGTGCAGCGATGGGGGCGGGGGGGGGGGGGGGG
CGCGCGCCAGGCGGGGCGGGGCGGGGCGAGGGGCGGGGCGGGGCGAGGCGG
AGAGGTGCGGCGGCAGCCAATCAGAGCGGCGCGCTCCGAAAGTTTCCTTTT
ATGGCGAGGCGGCGGCGGCGGCGGCCCTATAAAAAGCGAAGCGCGCGGCGG
GCGGGAGTCGCTGCGACGCTGCCTTCGCCCCGTGCCCCGCTCCGCCGCCGC
CTCGCGCCGCCCGCCCCGGCTCTGACTGACCGCGTTACTCCCACAGGTGAG
CGGGCGGGACGGCCCTTCTCCTCCGGGCTGTAATTAGCTGAGCAAGAGGTA
AGGGTTTAAGGGATGGTTGGTTGGTGGGGTATTAATGTTTAATTACCTGGA
GCACCTGCCTGAAATCACTTTTTTTCAGGTTGGACCGGTGCCACCATGAAA
CGGACAGCCGACGGAAGCGAGTTCGAGTCACCAAAGAAGAAGCGGAAAGTC
TCTGAAGTCGAGTTTAGCCACGAGTATTGGATGAGGCACGCACTGACCCTG
GCAAAGCGAGCATGGGATGAAAGAGAAGTCCCCGTGGGCGCCGTGCTGGTG
CACAACAATAGAGTGATCGGAGAGGGATGGAACAGGCCAATCGGCCGCCAC
GACCCTACCGCACACGCAGAGATCATGGCACTGAGGCAGGGAGGCCTGGTC
ATGCAGAATTACCGCCTGATCGATGCCACCCTGTATGTGACACTGGAGCCA
TGCGTGATGTGCGCAGGAGCAATGATCCACAGCAGGATCGGAAGAGTGGTG
TTCGGAGCACGGGACGCCAAGACCGGCGCAGCAGGCTCCCTGATGGATGTG
CTGCACCACCCCGGCATGAACCACCGGGTGGAGATCACAGAGGGAATCCTG
GCAGACGAGTGCGCCGCCCTGCTGAGCGATTTCTTTAGAATGCGGAGACAG
GAGATCAAGGCCCAGAAGAAGGCACAGAGCTCCACCGACTCTGGAGGATCT
AGCGGAGGATCCTCTGGAAGCGAGACACCAGGCACAAGCGAGTCCGCCACA
CCAGAGAGCTCCGGCGGCTCCTCCGGAGGATCCTCTGAGGTGGAGTTTTCC
CACGAGTACTGGATGAGACATGCCCTGACCCTGGCCAAGAGGGCACGCGAT
GAGAGGGAGGTGCCTGTGGGAGCCGTGCTGGTGCTGAACAATAGAGTGATC
GGCGAGGGCTGGAACAGAGCCATCGGCCTGCACGACCCAACAGCCCATGCC
GAAATTATGGCCCTGAGACAGGGCGGCCTGGTCATGCAGAACTACAGACTG
ATTGACGCCACCCTGTACGTGACATTCGAGCCTTGCGTGATGTGCGCCGGC
GCCATGATCCACTCTAGGATCGGCCGCGTGGTGTTTGGCGTGAGGAACGCA
AAAACCGGCGCCGCAGGCTCCCTGATGGACGTGCTGCACTACCCCGGCATG
AATCACCGCGTCGAAATTACCGAGGGAATCCTGGCAGATGAATGTGCCGCC
CTGCTGTGCTATTTCTTTCGGATGCCTAGACAGGTGTTCAATGCTCAGAAG
AAGGCCCAGAGCTCCACCGACTCCGGAGGATCTAGCGGAGGCTCCTCTGGC
TCTGAGACACCTGGCACAAGCGAGAGCGCAACACCTGAAAGCAGCGGGGGC
AGCAGCGGGGGGTCAGACAAGAAGTACAGCATCGGCCTGGCCATCGGCACC
AACTCTGTGGGCTGGGCCGTGATCACCGACGAGTACAAGGTGCCCAGCAAG
AAATTCAAGGTGCTGGGCAACACCGACCGGCACAGCATCAAGAAGAACCTG
ATCGGAGCCCTGCTGTTCGACAGCGGCGAAACAGCCGAGGCCACCCGGCTG
AAGAGAACCGCCAGAAGAAGATACACCAGACGGAAGAACCGGATCTGCTAT
CTGCAAGAGATCTTCAGCAACGAGATGGCCAAGGTGGACGACAGCTTCTTC
CACAGACTGGAAGAGTCCTTCCTGGTGGAAGAGGATAAGAAGCACGAGCGG
CACCCCATCTTCGGCAACATCGTGGACGAGGTGGCCTACCACGAGAAGTAC
CCCACCATCTACCACCTGAGAAAGAAACTGGTGGACAGCACCGACAAGGCC
GACCTGCGGCTGATCTATCTGGCCCTGGCCCACATGATCAAGTTCCGGGGC
CACTTCCTGATCGAGGGCGACCTGAACCCCGACAACAGCGACGTGGACAAG
CTGTTCATCCAGCTGGTGCAGACCTACAACCAGCTGTTCGAGGAAAACCCC
ATCAACGCCAGCGGCGTGGACGCCAAGGCCATCCTGTCTGCCAGACTGAGC
AAGAGCAGACGGCTGGAAAATCTGATCGCCCAGCTGCCCGGCGAGAAGAAG
AATGGCCTGTTCGGAAACCTGATTGCCCTGAGCCTGGGCCTGACCCCCAAC
TTCAAGAGCAACTTCGACCTGGCCGAGGATGCCAAACTGCAGCTGAGCAAG
GACACCTACGACGACGACCTGGACAACCTGCTGGCCCAGATCGGCGACCAG
TACGCCGACCTGTTTCTGGCCGCCAAGAACCTGTCCGACGCCATCCTGCTG
AGCGACATCCTGAGAGTGAACACCGAGATCACCAAGGCCCCCCTGAGCGCC
TCTATGATCAAGAGATACGACGAGCACCACCAGGACCTGACCCTGCTGAAA
GCTCTCGTGCGGCAGCAGCTGCCTGAGAAGTACAAAGAGATTTTCTTCGAC
CAGAGCAAGAACGGCTACGCCGGCTACATTGACGGCGGAGCCAGCCAGGAA
GAGTTCTACAAGTTCATCAAGCCCATCCTGGAAAAGATGGACGGCACCGAG
GAACTGCTCGTGAAGCTGAACAGAGAGGACCTGCTGCGGAAGCAGCGGACC
TTCGACAACGGCAGCATCCCCCACCAGATCCACCTGGGAGAGCTGCACGCC
ATTCTGCGGCGGCAGGAAGATTTTTACCCATTCCTGAAGGACAACCGGGAA
AAGATCGAGAAGATCCTGACCTTCCGCATCCCCTACTACGTGGGCCCTCTG
GCCAGGGGAAACAGCAGATTCGCCTGGATGACCAGAAAGAGCGAGGAAACC
ATCACCCCCTGGAACTTCGAGGAAGTGGTGGACAAGGGCGCTTCCGCCCAG
AGCTTCATCGAGCGGATGACCAACTTCGATAAGAACCTGCCCAACGAGAAG
GTGCTGCCCAAGCACAGCCTGCTGTACGAGTACTTCACCGTGTATAACGAG
CTGACCAAAGTGAAATACGTGACCGAGGGAATGAGAAAGCCCGCCTTCCTG
AGCGGCGAGCAGAAAAAGGCCATCGTGGACCTGCTGTTCAAGACCAACCGG
AAAGTGACCGTGAAGCAGCTGAAAGAGGACTACTTCAAGAAAATCGAGTGC
CTGTCCTACGAGACAGAGATCCTGACAGTGGAGTATGGCCTGCTGCCAATC
GGCAAGATCGTGGAGAAGAGGATCGAGTGTACCGTGTACTCTGTGGATAAC
AATGGCAACATCTATACACAGCCCGTGGCACAGTGGCACGATAGGGGAGAG
CAGGAGGTGTTCGAGTATTGCCTGGAGGACGGCAGCCTGATCAGGGCAACC
AAGGACCACAAGTTCATGACAGTGGATGGCCAGATGCTGCCCATCGACGAG
ATTTTCGAGCGGGAGCTGGACCTGATGAGAGTGGATAACCTGCCTAATAGC
GGAGGCAGTAAAAGAACAGCAGACGGGAGTGAGTTTGAGCCCAAGAAAAAG
AGAAAGGTGTAAGATCTGATAATCAACCTCTGGATTACAAAATTTGTGAAA
GATTGACTGGTATTCTTAACTATGTTGCTCCTTTTACGCTATGTGGATACG
CTGCTTTAATGCCTTTGTATCATGCTATTGCTTCCCGTATGGCTTTCATTT
TCTCCTCCTTGTATAAATCCTGGTTAGTTCTTGCCACGGCGGAACTCATCG
CCGCCTGCCTTGCCCGCTGCTGGACAGGGGCTCGGCTGTTGGGCACTGACA
ATTCCGTGGTGCGACTGTGCCTTCTAGTTGCCAGCCATCTGTTGTTTGCCC
CTCCCCCGTGCCTTCCTTGACCCTGGAAGGTGCCACTCCCACTGTCCTTTC
CTAATAAAATGAGGAAATTGCATCGCATTGTCTGAGTAGGTGTCATTCTAT
TCTGGGGGGGGGGTGGGGCAGGACAGCAAGGGGGAGGATTGGGAAGACAAT
AGCAGGCATGCTGGGGATGCGGTGGGCTCTATGGGCGGCCGCAGGAACCCC
TAGTGATGGAGTTGGCCACTCCCTCTCTGCGCGCTCGCTCGCTCACTGAGG
CCGGGCGACCAAAGGTCGCCCGACGCCCGGGCTTTGCCCGGGCGGCCTCAG
TGAGCGAGCGAGCGCGCAG

AAV vector sequence comprising ABE8e-SpCas9 amino acids 1-572 N-intein:

(SEQ ID NO: 109)
CTGCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGGGA
GTGGCCAACTCCATCACTAGGGGTTCCTGCGGCCTCTAGATCAGGGTACCC
GTTACATAACTTACGGTAAATGGCCCGCCTGGCTGACCGCCCAACGACCCC
CGCCCATTGACGTCAATAGTAACGCCAATAGGGACTTTCCATTGACGTCAA
TGGGTGGAGTATTTACGGTAAACTGCCCACTTGGCAGTACATCAAGTGTAT
CATATGCCAAGTACGCCCCCTATTGACGTCAATGACGGTAAATGGCCCGCC
TGGCATTGTGCCCAGTACATGACCTTATGGGACTTTCCTACTTGGCAGTAC
ATCTACGTATTAGTCATCGCTATTACCATGGTCGAGGTGAGCCCCACGTTC
TGCTTCACTCTCCCCATCTCCCCCCCCTCCCCACCCCCAATTTTGTATTTA
TTTATTTTTTAATTATTTTGTGCAGCGATGGGGGCGGGGGGGGGGGGGGGG
CGCGCGCCAGGCGGGGCGGGGCGGGGCGAGGGGCGGGGCGGGGCGAGGCGG
AGAGGTGCGGCGGCAGCCAATCAGAGCGGCGCGCTCCGAAAGTTTCCTTTT
ATGGCGAGGCGGCGGCGGCGGCGGCCCTATAAAAAGCGAAGCGCGCGGCGG
GCGGGAGTCGCTGCGACGCTGCCTTCGCCCCGTGCCCCGCTCCGCCGCCGC
CTCGCGCCGCCCGCCCCGGCTCTGACTGACCGCGTTACTCCCACAGGTGAG
CGGGCGGGACGGCCCTTCTCCTCCGGGCTGTAATTAGCTGAGCAAGAGGTA
AGGGTTTAAGGGATGGTTGGTTGGTGGGGTATTAATGTTTAATTACCTGGA
GCACCTGCCTGAAATCACTTTTTTTCAGGTTGGACCGGTGCCACCATGAAA
CGGACAGCCGACGGAAGCGAGTTCGAGTCACCAAAGAAGAAGCGGAAAGTC
TCTGAGGTGGAGTTTTCCCACGAGTACTGGATGAGACATGCCCTGACCCTG
GCCAAGAGGGCACGGGATGAGAGGGAGGTGCCTGTGGGAGCCGTGCTGGTG
CTGAACAATAGAGTGATCGGCGAGGGCTGGAACAGAGCCATCGGCCTGCAC
GACCCAACAGCCCATGCCGAAATTATGGCCCTGAGACAGGGCGGCCTGGTC
ATGCAGAACTACAGACTGATTGACGCCACCCTGTACGTGACATTCGAGCCT
TGCGTGATGTGCGCCGGCGCCATGATCCACTCTAGGATCGGCCGCGTGGTG
TTTGGCTGGAGGAACTCAAAAAGAGGCGCCGCAGGCTCCCTGATGAACGTG
CTGAACTACCCCGGCATGAATCACCGCGTCGAAATTACCGAGGGAATCCTG
GCAGATGAATGTGCCGCCCTGCTGTGCGATTTCTATCGGATGCCTAGACAG
GTGTTCAATGCTCAGAAGAAGGCCCAGAGCTCCATCAACTCCGGAGGATCT
AGCGGAGGCTCCTCTGGCTCTGAGACACCTGGCACAAGCGAGAGCGCAACA
CCTGAAAGCAGCGGGGGCAGCAGCGGGGGGTCAGACAAGAAGTACAGCATC
GGCCTGGCCATCGGCACCAACTCTGTGGGCTGGGCCGTGATCACCGACGAG
TACAAGGTGCCCAGCAAGAAATTCAAGGTGCTGGGCAACACCGACCGGCAC
AGCATCAAGAAGAACCTGATCGGAGCCCTGCTGTTCGACAGCGGCGAAACA
GCCGAGGCCACCCGGCTGAAGAGAACCGCCAGAAGAAGATACACCAGACGG
AAGAACCGGATCTGCTATCTGCAAGAGATCTTCAGCAACGAGATGGCCAAG
GTGGACGACAGCTTCTTCCACAGACTGGAAGAGTCCTTCCTGGTGGAAGAG
GATAAGAAGCACGAGCGGCACCCCATCTTCGGCAACATCGTGGACGAGGTG
GCCTACCACGAGAAGTACCCCACCATCTACCACCTGAGAAAGAAACTGGTG
GACAGCACCGACAAGGCCGACCTGCGGCTGATCTATCTGGCCCTGGCCCAC
ATGATCAAGTTCCGGGGCCACTTCCTGATCGAGGGCGACCTGAACCCCGAC
AACAGCGACGTGGACAAGCTGTTCATCCAGCTGGTGCAGACCTACAACCAG
CTGTTCGAGGAAAACCCCATCAACGCCAGCGGCGTGGACGCCAAGGCCATC
CTGTCTGCCAGACTGAGCAAGAGCAGACGGCTGGAAAATCTGATCGCCCAG
CTGCCCGGCGAGAAGAAGAATGGCCTGTTCGGAAACCTGATTGCCCTGAGC
CTGGGCCTGACCCCCAACTTCAAGAGCAACTTCGACCTGGCCGAGGATGCC
AAACTGCAGCTGAGCAAGGACACCTACGACGACGACCTGGACAACCTGCTG
GCCCAGATCGGCGACCAGTACGCCGACCTGTTTCTGGCCGCCAAGAACCTG
TCCGACGCCATCCTGCTGAGCGACATCCTGAGAGTGAACACCGAGATCACC
AAGGCCCCCCTGAGCGCCTCTATGATCAAGAGATACGACGAGCACCACCAG
GACCTGACCCTGCTGAAAGCTCTCGTGCGGCAGCAGCTGCCTGAGAAGTAC
AAAGAGATTTTCTTCGACCAGAGCAAGAACGGCTACGCCGGCTACATTGAC
GGCGGAGCCAGCCAGGAAGAGTTCTACAAGTTCATCAAGCCCATCCTGGAA
AAGATGGACGGCACCGAGGAACTGCTCGTGAAGCTGAACAGAGAGGACCTG
CTGCGGAAGCAGCGGACCTTCGACAACGGCAGCATCCCCCACCAGATCCAC
CTGGGAGAGCTGCACGCCATTCTGCGGCGGCAGGAAGATTTTTACCCATTC
CTGAAGGACAACCGGGAAAAGATCGAGAAGATCCTGACCTTCCGCATCCCC
TACTACGTGGGCCCTCTGGCCAGGGGAAACAGCAGATTCGCCTGGATGACC
AGAAAGAGCGAGGAAACCATCACCCCCTGGAACTTCGAGGAAGTGGTGGAC
AAGGGCGCTTCCGCCCAGAGCTTCATCGAGCGGATGACCAACTTCGATAAG
AACCTGCCCAACGAGAAGGTGCTGCCCAAGCACAGCCTGCTGTACGAGTAC
TTCACCGTGTATAACGAGCTGACCAAAGTGAAATACGTGACCGAGGGAATG
AGAAAGCCCGCCTTCCTGAGCGGCGAGCAGAAAAAGGCCATCGTGGACCTG
CTGTTCAAGACCAACCGGAAAGTGACCGTGAAGCAGCTGAAAGAGGACTAC
TTCAAGAAAATCGAGTGCCTGTCCTACGAGACAGAGATCCTGACAGTGGAG
TATGGCCTGCTGCCAATCGGCAAGATCGTGGAGAAGAGGATCGAGTGTACC
GTGTACTCTGTGGATAACAATGGCAACATCTATACACAGCCCGTGGCACAG
TGGCACGATAGGGGAGAGCAGGAGGTGTTCGAGTATTGCCTGGAGGACGGC
AGCCTGATCAGGGCAACCAAGGACCACAAGTTCATGACAGTGGATGGCCAG
ATGCTGCCCATCGACGAGATTTTCGAGCGGGAGCTGGACCTGATGAGAGTG
GATAACCTGCCTAATAGCGGAGGCAGTAAAAGAACAGCAGACGGGAGTGAG
TTTGAGCCCAAGAAAAAGAGAAAGGTGTAAGATCTGATAATCAACCTCTGG
ATTACAAAATTTGTGAAAGATTGACTGGTATTCTTAACTATGTTGCTCCTT
TTACGCTATGTGGATACGCTGCTTTAATGCCTTTGTATCATGCTATTGCTT
CCCGTATGGCTTTCATTTTCTCCTCCTTGTATAAATCCTGGTTAGTTCTTG
CCACGGCGGAACTCATCGCCGCCTGCCTTGCCCGCTGCTGGACAGGGGCTC
GGCTGTTGGGCACTGACAATTCCGTGGTGCGACTGTGCCTTCTAGTTGCCA
GCCATCTGTTGTTTGCCCCTCCCCCGTGCCTTCCTTGACCCTGGAAGGTGC
CACTCCCACTGTCCTTTCCTAATAAAATGAGGAAATTGCATCGCATTGTCT
GAGTAGGTGTCATTCTATTCTGGGGGGTGGGGTGGGGCAGGACAGCAAGGG
GGAGGATTGGGAAGACAATAGCAGGCATGCTGGGGATGCGGTGGGCTCTAT
GGGCGGCCGCAGGAACCCCTAGTGATGGAGTTGGCCACTCCCTCTCTGCGC
GCTCGCTCGCTCACTGAGGCCGGGCGACCAAAGGTCGCCCGACGCCCGGGC
TTTGCCCGGGCGGCCTCAGTGAGCGAGCGAGCGCGCAG

In certain embodiments, the first AAV particle contains a polynucleotide comprising the sequence of SEQ ID NO: 108 or 109. In some embodiments, the polynucleotide comprises one or more AAV inverted terminal repeat (ITR) sequences. In some embodiments, the polynucleotide comprises a promoter (e.g., a Cbh promoter). In some embodiments, the polynucleotide comprises a portion encoding an N-terminal portion of a base editor, such as ABE7.10-SpCas9 or ABE8e-SpCas9 (e.g., ABE7.10-SpCas9 amino acids 1-572 or ABE8e-SpCas9 amino acids 1-572). In some embodiments, the polynucleotide comprises an N-intein. In some embodiments, the polynucleotide comprises a post-transcriptional regulatory element (e.g., “W3,” the minimized gamma portion of the woodchuck hepatitis virus post-transcriptional regulatory element WPRE as described in Davis et al., Nature Biotechnology 2023, 42, 253-264, which is incorporated herein by reference). In certain embodiments, the polynucleotide comprises the structure 5′-[AAV ITR]-[promoter]-[N-terminal portion of base editor]-[N-intein]-[post-transcriptional regulatory element]-[AAV ITR]-3′.

In some embodiments, a second AAV particle contains a polynucleotide comprising a sequence at least 80%, at least 85%, at least 90%, at least 95%, at least 96%, at least 97%, at least 98%, or at least 99% identical to SEQ ID NO: 110: AAV vector sequence for C-intein SpCas9 amino acids 573-1367 and sgRNA

(SEQ ID NO: 110)
CTGCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGGGA
GTGGCCAACTCCATCACTAGGGGTTCCTGCGGCCTCTAGATCAGGGTACCC
GTTACATAACTTACGGTAAATGGCCCGCCTGGCTGACCGCCCAACGACCCC
CGCCCATTGACGTCAATAGTAACGCCAATAGGGACTTTCCATTGACGTCAA
TGGGTGGAGTATTTACGGTAAACTGCCCACTTGGCAGTACATCAAGTGTAT
CATATGCCAAGTACGCCCCCTATTGACGTCAATGACGGTAAATGGCCCGCC
TGGCATTGTGCCCAGTACATGACCTTATGGGACTTTCCTACTTGGCAGTAC
ATCTACGTATTAGTCATCGCTATTACCATGGTCGAGGTGAGCCCCACGTTC
TGCTTCACTCTCCCCATCTCCCCCCCCTCCCCACCCCCAATTTTGTATTTA
TTTATTTTTTAATTATTTTGTGCAGCGATGGGGGCGGGGGGGGGGGGGGGG
CGCGCGCCAGGCGGGGCGGGGGGGGCGAGGGGGGGGGGGGGGCGAGGCGGA
GAGGTGCGGCGGCAGCCAATCAGAGCGGCGCGCTCCGAAAGTTTCCTTTTA
TGGCGAGGCGGCGGCGGCGGCGGCCCTATAAAAAGCGAAGCGCGCGGCGGG
CGGGAGTCGCTGCGACGCTGCCTTCGCCCCGTGCCCCGCTCCGCCGCCGCC
TCGCGCCGCCCGCCCCGGCTCTGACTGACCGCGTTACTCCCACAGGTGAGC
GGGCGGGACGGCCCTTCTCCTCCGGGCTGTAATTAGCTGAGCAAGAGGTAA
GGGTTTAAGGGATGGTTGGTTGGTGGGGTATTAATGTTTAATTACCTGGAG
CACCTGCCTGAAATCACTTTTTTTCAGGTTGGACCGGTGCCACCATGAAAC
GGACAGCCGACGGAAGCGAGTTCGAGTCACCAAAGAAGAAGCGGAAAGTCA
TCAAGATTGCTACACGGAAATACCTGGGAAAGCAGAACGTGTACGACATCG
GCGTGGAGCGGGATCACAACTTCGCCCTGAAGAATGGCTTTATCGCCAGCA
ATTGCTTCGACTCCGTGGAAATCTCCGGCGTGGAAGATCGGTTCAACGCCT
CCCTGGGCACATACCACGATCTGCTGAAAATTATCAAGGACAAGGACTTCC
TGGACAATGAGGAAAACGAGGACATTCTGGAAGATATCGTGCTGACCCTGA
CACTGTTTGAGGACAGAGAGATGATCGAGGAACGGCTGAAAACCTATGCCC
ACCTGTTCGACGACAAAGTGATGAAGCAGCTGAAGCGGCGGAGATACACCG
GCTGGGGCAGGCTGAGCCGGAAGCTGATCAACGGCATCCGGGACAAGCAGT
CCGGCAAGACAATCCTGGATTTCCTGAAGTCCGACGGCTTCGCCAACAGAA
ACTTCATGCAGCTGATCCACGACGACAGCCTGACCTTTAAAGAGGACATCC
AGAAAGCCCAGGTGTCCGGCCAGGGCGATAGCCTGCACGAGCACATTGCCA
ATCTGGCCGGCAGCCCCGCCATTAAGAAGGGCATCCTGCAGACAGTGAAGG
TGGTGGACGAGCTCGTGAAAGTGATGGGCCGGCACAAGCCCGAGAACATCG
TGATCGAAATGGCCAGAGAGAACCAGACCACCCAGAAGGGACAGAAGAACA
GCCGCGAGAGAATGAAGCGGATCGAAGAGGGCATCAAAGAGCTGGGCAGCC
AGATCCTGAAAGAACACCCCGTGGAAAACACCCAGCTGCAGAACGAGAAGC
TGTACCTGTACTACCTGCAGAATGGGGGGATATGTACGTGGACCAGGAACT
GGACATCAACCGGCTGTCCGACTACGATGTGGACCATATCGTGCCTCAGAG
CTTTCTGAAGGACGACTCCATCGACAACAAGGTGCTGACCAGAAGCGACAA
GAACCGGGGCAAGAGCGACAACGTGCCCTCCGAAGAGGTCGTGAAGAAGAT
GAAGAACTACTGGCGGCAGCTGCTGAACGCCAAGCTGATTACCCAGAGAAA
GTTCGACAATCTGACCAAGGCCGAGAGAGGCGGCCTGAGCGAACTGGATAA
GGCCGGCTTCATCAAGAGACAGCTGGTGGAAACCCGGCAGATCACAAAGCA
CGTGGCACAGATCCTGGACTCCCGGATGAACACTAAGTACGACGAGAATGA
CAAGCTGATCCGGGAAGTGAAAGTGATCACCCTGAAGTCCAAGCTGGTGTC
CGATTTCCGGAAGGATTTCCAGTTTTACAAAGTGCGCGAGATCAACAACTA
CCACCACGCCCACGACGCCTACCTGAACGCCGTCGTGGGAACCGCCCTGAT
CAAAAAGTACCCTAAGCTGGAAAGCGAGTTCGTGTACGGCGACTACAAGGT
GTACGACGTGCGGAAGATGATCGCCAAGAGCGAGCAGGAAATCGGCAAGGC
TACCGCCAAGTACTTCTTCTACAGCAACATCATGAACTTTTTCAAGACCGA
GATTACCCTGGCCAACGGCGAGATCCGGAAGCGGCCTCTGATCGAGACAAA
CGGCGAAACCGGGGAGATCGTGTGGGATAAGGGCCGGGATTTTGCCACCGT
GCGGAAAGTGCTGAGCATGCCCCAAGTGAATATCGTGAAAAAGACCGAGGT
GCAGACAGGCGGCTTCAGCAAAGAGTCTATCCTGCCCAAGAGGAACAGCGA
TAAGCTGATCGCCAGAAAGAAGGACTGGGACCCTAAGAAGTACGGCGGCTT
CGACAGCCCCACCGTGGCCTATTCTGTGCTGGTGGTGGCCAAAGTGGAAAA
GGGCAAGTCCAAGAAACTGAAGAGTGTGAAAGAGCTGCTGGGGATCACCAT
CATGGAAAGAAGCAGCTTCGAGAAGAATCCCATCGACTTTCTGGAAGCCAA
GGGCTACAAAGAAGTGAAAAAGGACCTGATCATCAAGCTGCCTAAGTACTC
CCTGTTCGAGCTGGAAAACGGCCGGAAGAGAATGCTGGCCTCTGCCGGCGA
ACTGCAGAAGGGAAACGAACTGGCCCTGCCCTCCAAATATGTGAACTTCCT
GTACCTGGCCAGCCACTATGAGAAGCTGAAGGGCTCCCCCGAGGATAATGA
GCAGAAACAGCTGTTTGTGGAACAGCACAAGCACTACCTGGACGAGATCAT
CGAGCAGATCAGCGAGTTCTCCAAGAGAGTGATCCTGGCCGACGCTAATCT
GGACAAAGTGCTGTCCGCCTACAACAAGCACCGGGATAAGCCCATCAGAGA
GCAGGCCGAGAATATCATCCACCTGTTTACCCTGACCAATCTGGGAGCCCC
TGCCGCCTTCAAGTACTTTGACACCACCATCGACCGGAAGAGGTACACCAG
CACCAAAGAGGTGCTGGACGCCACCCTGATCCACCAGAGCATCACCGGCCT
GTACGAGACACGGATCGACCTGTCTCAGCTGGGAGGTGACTCTGGCGGCTC
AAAAAGAACCGCCGACGGCAGCGAATTCGAGCCCAAGAAGAAGAGGAAAGT
CTAAGATCTGATAATCAACCTCTGGATTACAAAATTTGTGAAAGATTGACT
GGTATTCTTAACTATGTTGCTCCTTTTACGCTATGTGGATACGCTGCTTTA
ATGCCTTTGTATCATGCTATTGCTTCCCGTATGGCTTTCATTTTCTCCTCC
TTGTATAAATCCTGGTTAGTTCTTGCCACGGCGGAACTCATCGCCGCCTGC
CTTGCCCGCTGCTGGACAGGGGCTCGGCTGTTGGGCACTGACAATTCCGTG
GTGCGACTGTGCCTTCTAGTTGCCAGCCATCTGTTGTTTGCCCCTCCCCCG
TGCCTTCCTTGACCCTGGAAGGTGCCACTCCCACTGTCCTTTCCTAATAAA
ATGAGGAAATTGCATCGCATTGTCTGAGTAGGTGTCATTCTATTCTGGGGG
GTGGGGTGGGGCAGGACAGCAAGGGGGAGGATTGGGAAGACAATAGCAGGC
ATGCTGGGGATGCGGTGGGCTCTATGGCTCGAGAAAAAAAGCACCGACTCG
GTGCCACTTTTTCAAGTTGATAACGGACTAGCCTTATTTTAACTTGCTATT
TCTAGCTCTAAAACTCTGTGATCCGTAAGTGATACGGTGTTTCGTCCTTTC
CACAAGATATATAAAGCCAAGAAATCGAAATACTTTCAAGTTACGGTAAGC
ATATGATAGTCCATTTTAAAACATAATTTTAAAACTGCAAACTACCCAAGA
AATTATTACTTTCTACGTCACGTATTTTGTACTAATATCTTTGTGTTTACA
GTCAAATTAATTCTAATTATCTCTCTAACAGCCTTGTATCGTATATGCAAA
TATGAAGGAATCATGGGAAATAGGCCCTCTTCCTGCCCGACCTTGCGGCCG
CAGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCGG
GCGGCCTCAGTGAGCGAGCGAGCGCGCAG

In certain embodiments, the second AAV particle contains a polynucleotide comprising the sequence of SEQ ID NO: 110. In some embodiments, the polynucleotide comprises one or more AAV inverted terminal repeat (ITR) sequences. In some embodiments, the polynucleotide comprises a promoter (e.g., a Cbh promoter). In some embodiments, the polynucleotide comprises a C-intein. In some embodiments, the polynucleotide comprises a portion encoding a C-terminal portion of a base editor, such as ABE7.10-SpCas9 or ABE8e-SpCas9 (e.g., SpCas9 amino acids 573-1367). In some embodiments, the polynucleotide comprises a post-transcriptional regulatory element (e.g., “W3,” the minimized gamma portion of the woodchuck hepatitis virus post-transcriptional regulatory element WPRE as described in Davis et al., Nature Biotechnology 2023, 42, 253-264, which is incorporated herein by reference). In some embodiments, the polynucleotide comprises a portion encoding an sgRNA. In certain embodiments, the polynucleotide comprises a promoter for expression of the sgRNA (e.g., an hU6 promoter). In certain embodiments, the polynucleotide comprises the structure 5′-[AAV ITR]-[promoter]-[C-intein]-[C-terminal portion of base editor]-[post-transcriptional regulatory element]-[promoter]-[sgRNA-]-[AAV ITR]-3′.

### Pharmaceutical Compositions

Other aspects of the present disclosure relate to pharmaceutical compositions comprising any of the gRNAs, base editors, complexes, AAV particles, polynucleotides, vectors, and/or cells described herein. The term “pharmaceutical composition,” as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

As used here, the term “pharmaceutically-acceptable carrier” (or “pharmaceutically acceptable excipient”) means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservatives, and antioxidants can also be present in the formulation. Terms such as “excipient,” “carrier,” “pharmaceutically acceptable carrier,” “pharmaceutically acceptable excipient,” or the like are used interchangeably herein.

In some embodiments, the pharmaceutical composition is formulated for delivery to a subject for gene editing (e.g., base editing).

The pharmaceutical compositions described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

In some embodiments, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease and may have a sterile access port. For example, the container may be an intravenous solution bag or a vial having a stopper pierce-able by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Polynucleotides, Vectors, Cells, and Kits

The present disclosure provides, in some aspects, polynucleotides and vectors encoding any of the gRNAs, base editors, complexes, and/or AAV particles described herein. In some aspects, the present disclosure provides polynucleotides and vectors encoding a gRNA and a base editor as disclosed herein. In some embodiments, the polynucleotides and vectors provided herein comprise DNA (e.g., plasmid DNA or viral DNA). In some embodiments, the polynucleotides and vectors provided herein comprise RNA (e.g., mRNA or viral RNA).

Cells that may contain any of the gRNAs, base editors, complexes, AAV particles, polynucleotides, and/or vectors described herein are also provided by the present disclosure. The methods described herein may be used to deliver a gRNA and base editor into a eukaryotic cell (e.g., a mammalian cell, such as a human cell). In some embodiments, the cell is in vitro (e.g., a cultured cell). In some embodiments, the cell is in vivo (e.g., in a subject, such as a human subject). In some embodiments, the cell is ex vivo (e.g., isolated from a subject and may be administered back to the same or a different subject).

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. In some embodiments, a cell transfected with one or more vectors described herein is used to establish a new cell line comprising one or more vector-derived sequences. In some embodiments, a cell transiently transfected with the components of a base editing system as described herein (such as by transient transfection of one or more vectors, or transfection with RNA), and modified through the activity of a base editing complex, is used to establish a new cell line comprising cells containing the modification but lacking any other exogenous sequence.

The gRNAs, base editors, complexes, AAV particles, polynucleotides, and/or vectors described herein may also be assembled into kits. In some embodiments, the kit comprises polynucleotides for expression of the gRNAs, base editors, complexes, and/or AAV particles described herein. In some embodiments, the kit comprises appropriate gRNAs or nucleic acid vectors for the expression of such gRNAs to target the Cas9 protein of a base editor to a desired target sequence, e.g., in Tpp1. In some embodiments, the gRNAs in the kit are useful for correcting an R208X mutation in a Tpp1 enzyme, where X is a premature stop codon.

The kits described herein may include one or more containers housing components for performing the methods described herein, and optionally instructions for use. Any of the kits described herein may further comprise components needed for performing the base editing methods described herein. Each component of the kits, where applicable, may be provided in liquid form (e.g., in solution) or in solid form, (e.g., a dry powder). In certain cases, some of the components may be reconstitutable or otherwise processible (e.g., to an active form), for example, by the addition of a suitable solvent or other species (for example, water), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use, or sale of pharmaceuticals or biological products, which can also reflect approval by the agency of manufacture, use, or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral, and electronic communication of any form, associated with the disclosure. Additionally, the kits may include other components depending on the specific application, as described herein.

The kits may contain any one or more of the components described herein in one or more containers. The components may be prepared sterilely, packaged in a syringe, and shipped refrigerated. Alternatively, they may be housed in a vial or other container for storage. A second container may have other components prepared sterilely. Alternatively, the kits may include the active agents premixed and shipped in a vial, tube, or other container.

The kits may have a variety of forms, such as a blister pouch, a shrink-wrapped pouch, a vacuum sealable pouch, a sealable thermoformed tray, or a similar pouch or tray form, with the accessories loosely packed within the pouch, one or more tubes, containers, a box, or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc.

### EXAMPLES

### Example 1. Base Editing for the Treatment of Batten Disease

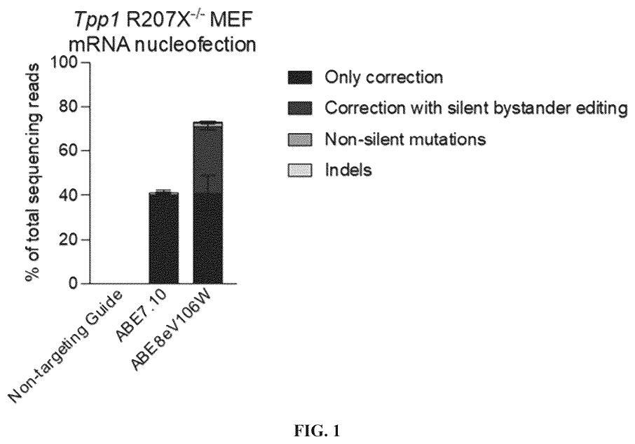

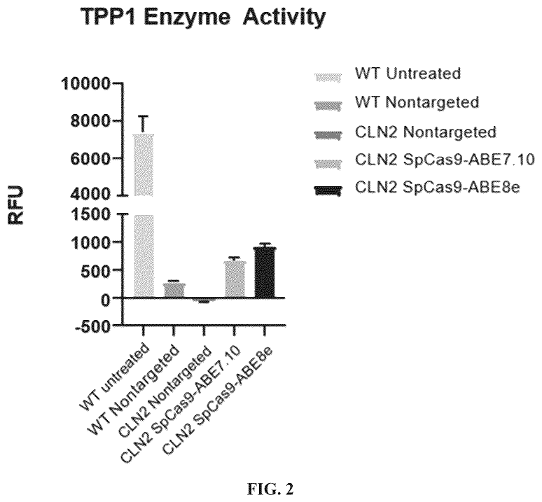

An editing strategy for reversion of the pathogenic mutation R208X in human Tpp1 (R207X in mouse Tpp1) was developed. Correction of mouse Tpp1 R207X with adenosine base editors (ABEs) in vitro was investigated. FIG. 1 shows the percent of total reads bearing X207R (reversion of pathogenic mutation) three days following electroporation (Lonza nucleofection) of SpCas9-based ABEs and an sgRNA targeting the protospacer TATCACTGACGGAGCACAGA (SEQ ID NO: 1) accompanied by silent or non-silent bystander mutations (FIG. 1). FIG. 2 shows that adenine base editing of mouse embryonic fibroblasts derived from the R207X−/− mouse model of CLN2 Batten disease partially restores Tpp1 enzyme activity in an editing efficiency-dependent manner.

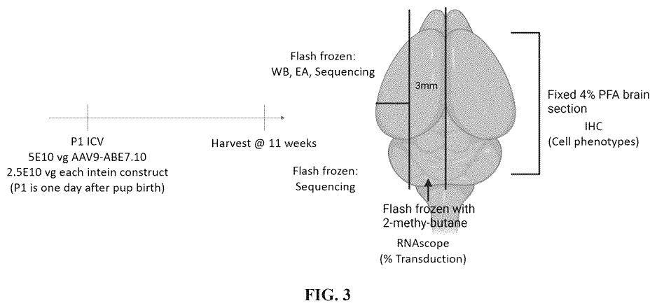

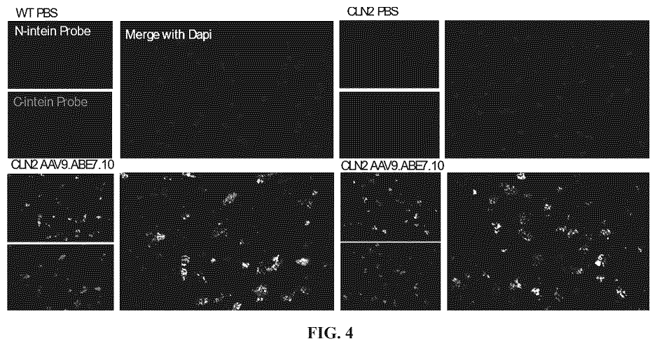

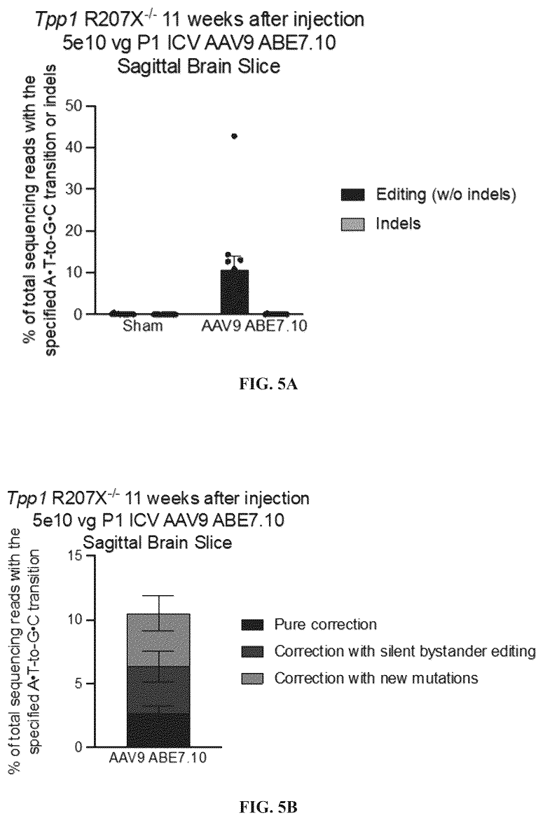

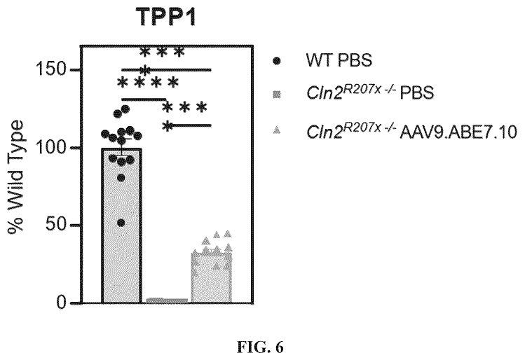

Editing efficiency was also assessed in vivo. FIG. 3 shows that adenine base editing of mouse embryonic fibroblasts derived from the R207X−/− mouse model of CLN2 Batten disease partially restores Tpp1 enzyme activity in an editing efficiency-dependent manner. In vivo RNAscope was also performed to assess AAV delivery of base editor and gRNA (FIG. 4). Green fluorescence indicates successful expression of N-intein viral construct, and magenta indicates successful expression of C-intein viral construct. Imaging indicated successful co-expression of the two constructs within cells in the brain 11 weeks following injection (FIG. 4). The ABE7.10 strategy was found to achieve 10% correction in bulk brain tissue in mice (FIG. 5A). Correction is associated with silent and non-silent bystander mutations to the displayed degrees (FIG. 5B). The main non-silent bystander edit is Y208H. In vivo editing was found to partially restore Tpp1 enzyme activity in tissues isolated from treated mice (FIG. 6).

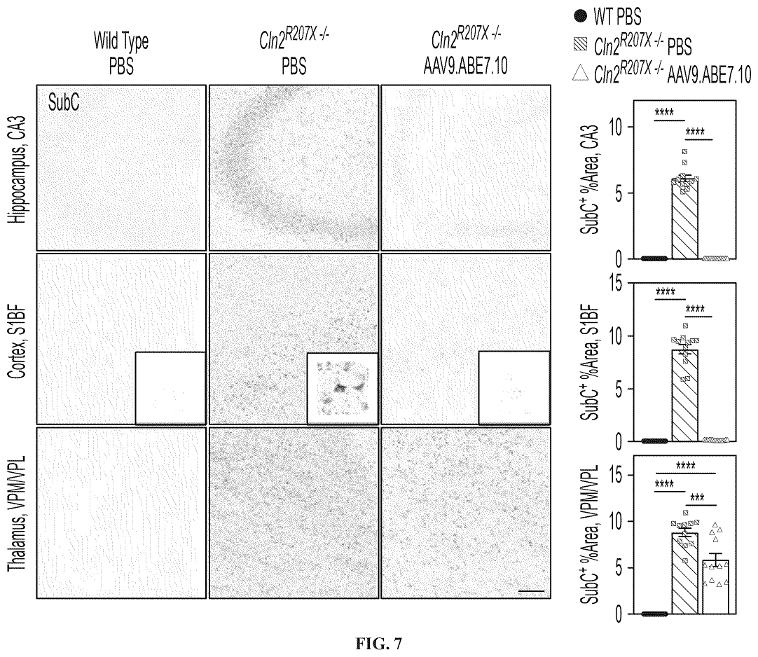

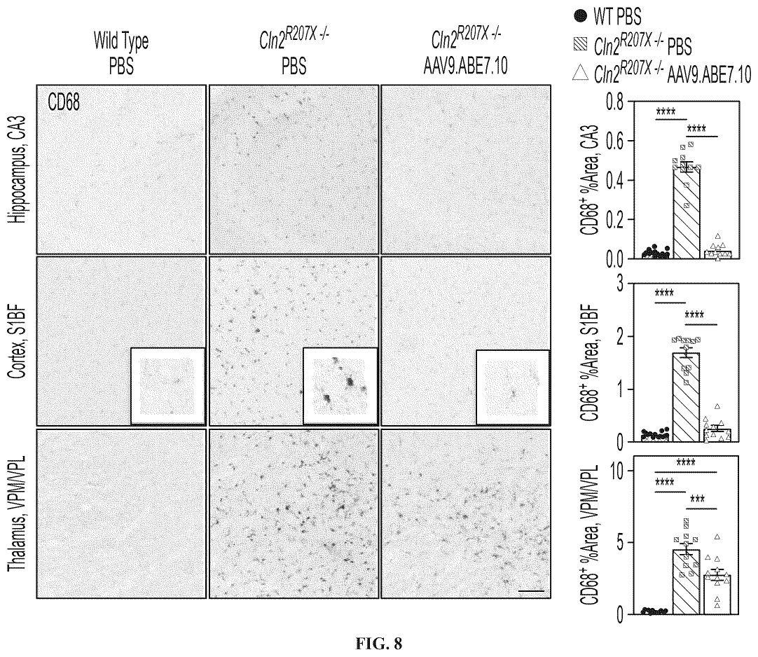

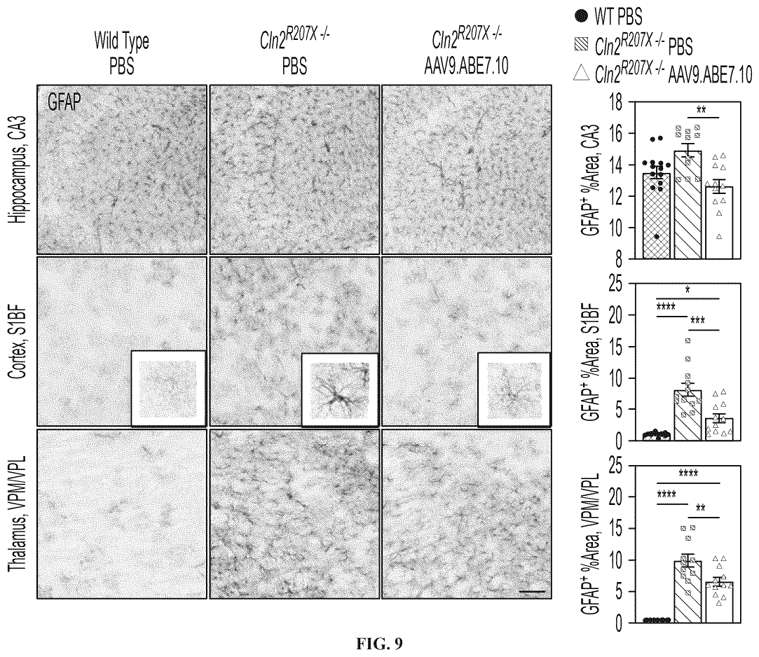

Editing was also found to abolish ATP synthase subunit C accumulation (a biomarker of degeneration resulting from Tpp1 R207X) in the hippocampus and cortex of mice, and significantly reduced it in the thalamus (FIG. 7). Editing reduced CD68 expression (a biomarker of microgliosis/degeneration resulting from Tpp1 R207X) to WT levels in the hippocampus and cortex of mice, and significantly reduced it in the thalamus (FIG. 8). Finally, editing reduced GFAP expression (a biomarker of astrocytosis/degeneration resulting from Tpp1 R207X) to wild type levels in the hippocampus, and significantly reduced it in the cortex and thalamus (FIG. 9).

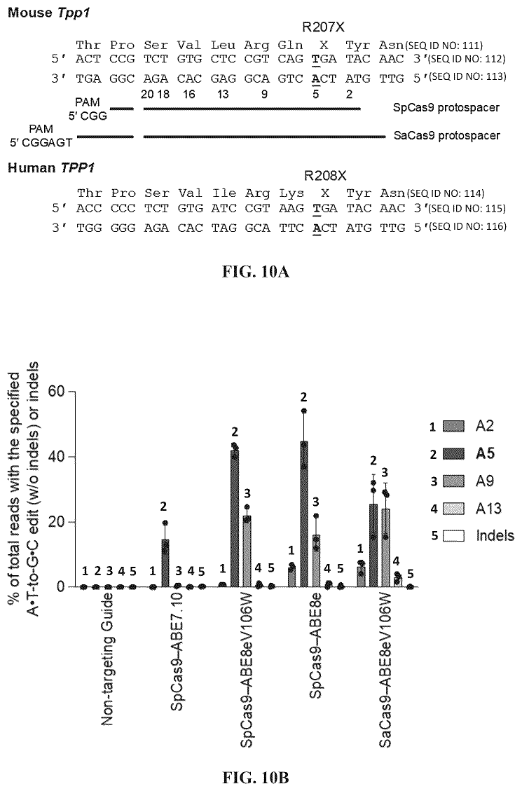

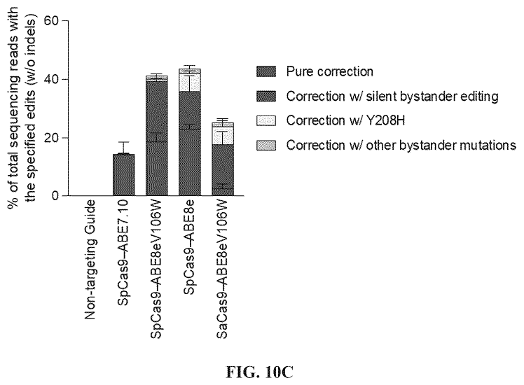

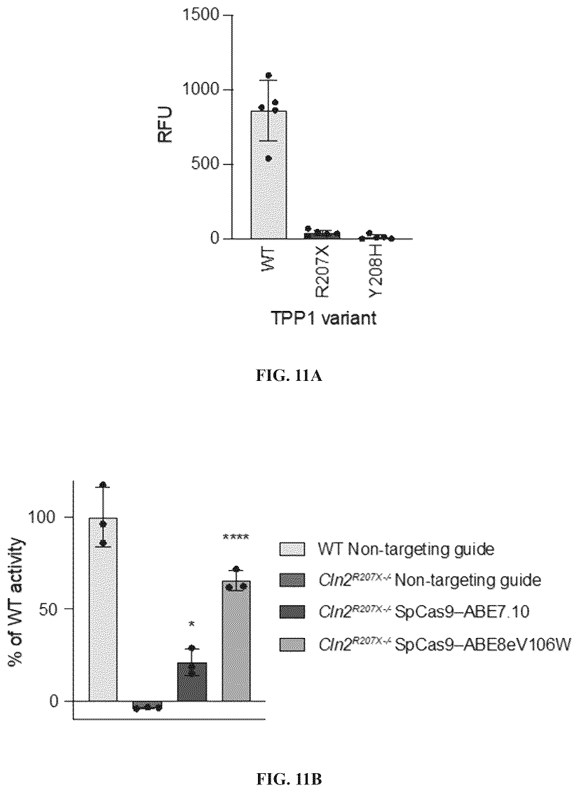

Adenine base editors were further evaluated for the correction of Tpp1 R207X (FIGS. 10A-10C). Adenines within the protospacer sequence (including the target mutant adenine) were targeted by the evaluated ABE protospacers as described herein (FIG. 10A). Percent editing efficiency at Tpp1 was measured by high-throughput sequencing of gDNA from Cln2^{R207X-/-} mouse embryonic fibroblasts (MEFs) 48 hours post electroporation with ABE mRNA and an sgRNA targeting the corresponding protospacer (FIG. 10B). SpCas9-ABE7.10 mRNA and a non-targeting sgRNA were electroporated for the non-targeting condition. Allele frequencies of the ABE-treated Cln2^{R207X-/-} MEF gDNA in FIG. 10B were also assessed (FIG. 10C). TPP1 enzyme activity was also characterized following adenine base editing (FIGS. 11A-11B). TPP1 enzyme activity of the major allele products generated by targeting Tpp1 R207X with adenine base editors (ABEs) was assessed (FIG. 11A). Following transfection with plasmids encoding the specified TPP1 variants, Neuro2A cells were lysed after 48 hours in culture and assayed for TPP1 activity. TPP1 activity in C1n2^{R207X-/-} mouse embryonic fibroblasts (MEFs) was characterized 48 hours post electroporation with the specified ABE mRNA and an sgRNA targeting Tpp1 R207X (FIG. 11B). SpCas9-ABE7.10 mRNA and a non-targeting sgRNA were electroporated for the non-targeted conditions.

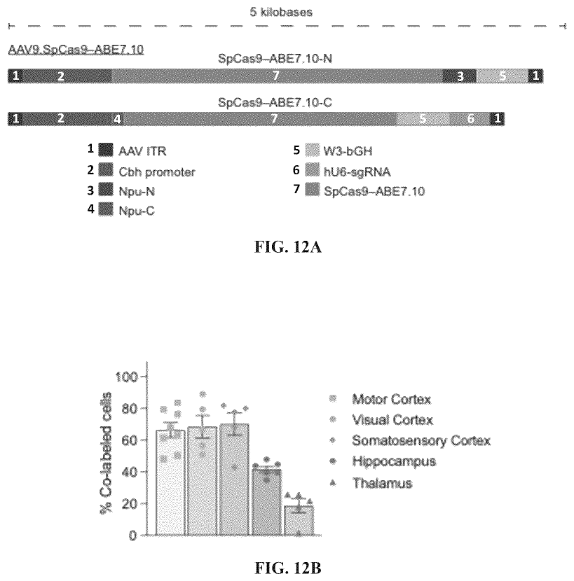

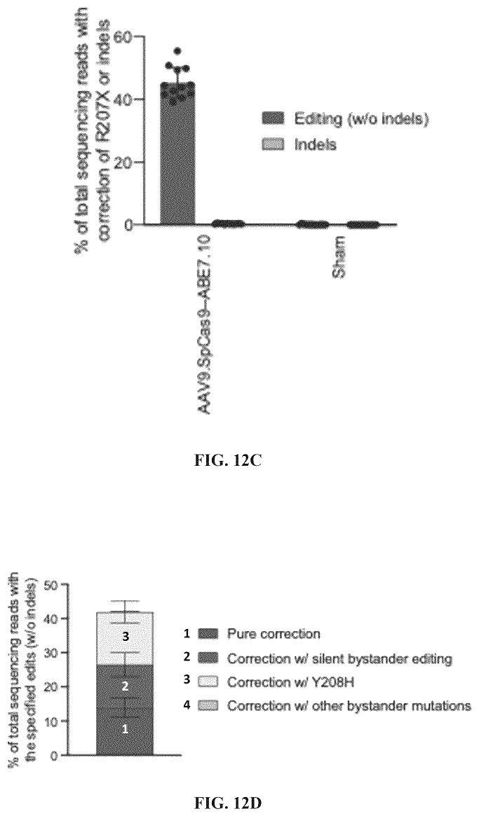

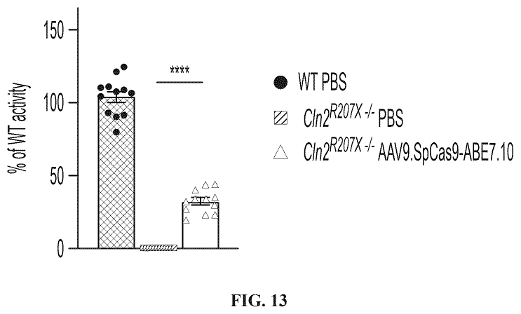

Next, efficiency of viral transduction and adenine base editing from a single injection of dual-AAV9 ABEs in C1n2^{R207X-/-} mice was assessed (FIGS. 12A-12D). A dual-vector AAV9.SpCas9-ABE7.10 architecture for correction of Tpp1 R207X was developed (FIG. 12A). Co-transduction efficiencies for AAV9.SpCas9-ABE7.10 and AAV9.SpCas9-ABE8eV106W in the cortex, hippocampus, and thalamus were assessed 11 weeks after a single ICV injection of 5×10¹⁰ vg (2.5×10¹⁰ vg each intein half) into P1 Cln2^{R207X-/-} mice (FIG. 12B). Flash-frozen brain sections were imaged by RNAScope using probes specific to either the N-intein- or C-intein-bearing construct to detect ABE expression. Bulk cortical gDNA editing efficiency was also measured by high-throughput sequencing of Tpp1 R207X (FIG. 12C), and allele frequencies of the ABE-treated Cln2^{R207X-/-} cortical gDNA in FIG. 12C were assessed (FIG. 12D). Finally, TPP1 enzyme activity was assessed after AA9.SpCas9-ABE7.10 treatment (FIG. 13). TPP1 activity from bulk cortical lysates was determined 11 weeks after a P1 intracerebroventricular (ICV) injection of AAV9.SpCas9-ABE7.10 (5×10¹⁰ vg total, 2.5×10¹⁰ vg each intein half) or PBS.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of base editing a tripeptidyl-peptidase 1 (Tpp1) gene comprising contacting a nucleic acid sequence encoding the Tpp1 gene with a base editor and a guide RNA (gRNA) targeting the base editor to the Tpp1 gene.

2. The method of claim 1, wherein the gRNA targets a strand complementary to a protospacer in a Tpp1 gene comprising a nucleotide sequence at least 80% identical to the sequence TATCACTGACGGAGCACAGA (SEQ ID NO: 1), TATCACTTACGGATCACAGA (SEQ ID NO: 2), or a fragment thereof.

3-4. (canceled)

5. The method of claim 1, wherein the gRNA comprises a spacer at least 80% identical to the sequence GTATCACTGACGGAGCACAGA (SEQ ID NO: 3) or GTATCACTTACGGATCACAGA (SEQ ID NO: 4).

6-8. (canceled)

9. The method of claim 1, wherein the gRNA comprises a sequence at least 80%, identical to the sequence
(SEQ ID NO: 6)
GTATCACTTACGGATCACAGAGTTTTAGAGCTAGAAATAGCAAGTTAAAA
TAAGGCTAGTCCGTTATCAACTTGAAAAAGTGGCACCGAGTCGGTGC
or
(SEQ ID NO: 7)
GTATCACTGACGGAGCACAGAGTTTTAGAGCTAGAAATAGCAAGTTAAAA
TAAGGCTAGTCCGTTATCAACTTGAAAAAGTGGCACCGAGTCGGTGC.

10-23. (canceled)

24. The method of claim 1, wherein the base editor comprises a deaminase comprising a sequence at least 80% identical to SEQ ID NO: 73 or 76.

25-27. (canceled)

28. The method of claim 1, wherein the base editor is ABE7.10, ABE8e, ABE8e(V106W), or a variant thereof.

29. The method of claim 1, wherein the base editor comprises a sequence at least 80% identical to any one of SEQ ID NOs: 105-107.

30-32. (canceled)

33. The method of claim 1, wherein one or more polynucleotides encoding the gRNA and the base editor are delivered to a cell comprising the nucleic acid sequence encoding the Tpp1 gene in one or more AAV particles, wherein the polynucleotide encoding the base editor is split between a first and a second AAV particle.

34-36. (canceled)

37. The method of claim 33, wherein the first AAV particle contains a polynucleotide comprising a sequence at least 80% identical to SEQ ID NO: 108 or 109, and wherein the second AAV particle contains a polynucleotide comprising a sequence at least 80% identical to SEQ ID NO: 110.

38-40. (canceled)

41. The method of claim 1, wherein the step of contacting corrects a C·G-to-T·A transition mutation in the Tpp1 gene.

42. The method of claim 41, wherein correction of the C·G-to-T·A transition mutation in the Tpp1 gene results in correction of an R208X mutation in a Tpp1 protein of SEQ ID NO: 9 or an R207X mutation in a Tpp1 protein of SEQ ID NO: 11, wherein X is a premature stop codon.

43-54. (canceled)

55. The method of claim 1, wherein the method is a method of treating Tpp1 R208X-mediated Batten disease in a subject.

56-57. (canceled)

58. A guide RNA (gRNA) targeting a strand complementary to a protospacer in a Tpp1 gene comprising a nucleotide sequence at least 80% identical to the sequence TATCACTGACGGAGCACAGA (SEQ ID NO: 1), TATCACTTACGGATCACAGA (SEQ ID NO: 2).

59-66. (canceled)

67. A complex comprising a gRNA of claim 58 and a base editor.

68-89. (canceled)

90. One or more AAV particles comprising one or more polynucleotides encoding the gRNA and the base editor, or a portion thereof, of the complex of claim 67.

91-106. (canceled)

107. A polynucleotide encoding the gRNA of claim 58.

108-109. (canceled)

110. A vector comprising the polynucleotide of claim 107.

111. A pharmaceutical composition comprising the one or more AAV particles of claim 90.

112-120. (canceled)

121. The method of claim 1, wherein the method is a method of correcting an R208X mutation in a tripeptidyl-peptidase 1 (Tpp1) gene, wherein X is a premature stop codon, and wherein the base editor is an ABE7.10 base editor and the guide RNA (gRNA) comprises the sequence of SEQ ID NO: 6.

122. The A-method of claim 1, wherein the method is a method of correcting an R208X mutation in a tripeptidyl-peptidase 1 (Tpp1) gene, wherein X is a premature stop codon, wherein the base editor is an ABE7.10 base editor and the guide RNA (gRNA) comprises the sequence of SEQ ID NO: 6, wherein the nucleic acid sequence encoding the Tpp1 gene is in a cell, and wherein the contacting comprises delivering a first AAV particle containing a nucleotide sequence of SEQ ID NO: 108 and a second AAV particle containing a nucleotide sequence of SEQ ID NO: 110 to the cell.

123-127. (canceled)
